(19) United States
(12) Patent Application Publication
(10) Pub. No.: US 2026/0198496 A1
(43) Pub. Date: Jul. 16, 2026

(54) AMIDE COMPOUND AND HARMFUL ARTHROPOD CONTROL COMPOSITION CONTAINING SAME

(71) Applicant: SUMITOMO CHEMICAL COMPANY, LIMITED, Tokyo (JP)

(72) Inventors: Yasushi KATAGIRI, Takarazuka-shi (JP); Mayumi OKAJIMA, Takarazuka-shi (JP); Yusuke NAMBA, Takarazuka-shi (JP)

(73) Assignee: SUMITOMO CHEMICAL COMPANY, LIMITED, Tokyo (JP)

(21) Appl. No.: 19/136,707

(22) PCT Filed: Dec. 25, 2023
(86) PCT No.: PCT/JP2023/046350

§ 371 Date: Jun. 6, 2025

(30) Foreign Application Priority Data
Jan. 5, 2023 (JP) 2023-000414

(51) Int. Cl. A01N 43/50; A01P 7/04; C07D 413/06

(52) U.S. Cl. CPC A01N 43/50; A01P 7/04; C07D 413/06

(57) ABSTRACT

The present invention provides a compound having an excellent control efficacy against harmful arthropods.

A compound represented by formula (I) [wherein R¹ represents a C1-C6 chain hydrocarbon group or the like; R², R³, R⁵, R⁶, and R⁷ are identical to or different from each other, and each represent a hydrogen atom or the like; R⁴ represents a C1-C6 chain hydrocarbon group or the like; R⁸ and R⁹ are identical to or different from each other, and each represent a hydrogen atom or the like; m represents 0, 1, 2, or 3; n represents 1, 2, 3, or 4; R¹⁰ represents a hydrogen atom or the like; Y represents a single bond, an oxygen atom, or —S(O)ᵤ—; when Y is a single bond, p represents 0; Q represents a C1-C8 chain hydrocarbon group or the like; and when Y is an oxygen atom or —S(O)ᵤ—, p represents 0, 1, 2, 3, 4, 5, 6, or 7; Q represents a C1-C8 chain hydrocarbon group or the like; u represents 0, 1, or 2; and R¹¹ and R¹² are identical to or different from each other, and each represent a hydrogen atom or the like] or a salt thereof has an excellent efficacy against harmful arthropods.

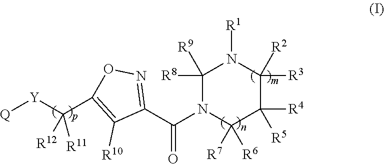

### TECHNICAL FIELD

This patent application claims the priority to and the benefit of Japanese Patent Application No. 2023-000414 (filed on Jan. 5, 2023), the entire contents of which are incorporated herein by reference.

The present invention relates to an amide compound and a harmful arthropod control composition containing the same.

### BACKGROUND ART

Various compounds have been investigated so far for controlling harmful arthropods. For example, Patent Document 1 describes that certain compounds have a harmful organism control effect.

### CITATION LIST

### Patent Document

Patent Document 1: WO 2014/119696 A

### SUMMARY OF THE INVENTION

### Problems to be Solved by the Invention

An object of the present invention is to provide a compound having an excellent control efficacy against harmful arthropods.

### Means for Solving the Problems

The present invention is as follows.

[1]A compound represented by formula (I)

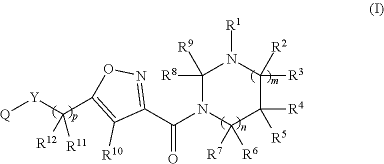

[wherein:
R¹ represents a C1-C6 chain hydrocarbon group optionally having one or more substituents selected from Group A, a C3-C6 alicyclic hydrocarbon group optionally having one or more substituents selected from Group A, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom;
R², R³, R⁵, R⁶, and R⁷ are identical to or different from each other, and each represent a C1-C6 chain hydrocarbon group optionally having one or more substituents selected from Group A, a C3-C6 alicyclic hydrocarbon group optionally having one or more substituents selected from Group A, a C1-C3 alkoxy group optionally having one or more halogen atoms, or a hydrogen atom;
R⁴ represents a C1-C6 chain hydrocarbon group optionally having one or more substituents selected from Group A, a C3-C6 alicyclic hydrocarbon group optionally having one or more substituents selected from Group A, or a C1-C3 alkoxy group optionally having one or more halogen atoms;
R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, a phenyl group, a pyridyl group, a furyl group, a tetrahydrofuryl group, a thienyl group, a tetrahydrothienyl group, a pyridazinyl group, a pyrimidinyl group, a pyrazinyl group {the phenyl group, the pyridyl group, the furyl group, the tetrahydrofuryl group, the thienyl group, the tetrahydrothienyl group, the pyridazinyl group, the pyrimidinyl group, and the pyrazinyl group may have one or more substituents selected from Group B}, or a hydrogen atom;
m represents 0, 1, 2, or 3;
when m is 2 or 3, R² and R³ of 2 or 3 may be each independently identical to or different from each other;
n represents 1, 2, 3, or 4,
when n is 2, 3, or 4, R⁶ and R⁷ of 2, 3, or 4 may be each independently identical to or different from each other;
R² and R³ may be combined to form —(CR¹³R¹⁴)—(CR¹⁵R¹⁶)ₐ—(CR¹⁷R¹⁸)—, or —(CR¹⁹R²⁰)_{b}—Z¹— (CR²¹R²²)_{c}—;
R⁴ and R⁵ may be combined to form —(CR²³R²⁴)—(CR²⁵R²⁶)_{d}—(CR²⁷R²⁸)—, or —(CR²⁹R³⁰)ₑ—Z²— (CR³¹R³²)_{f}—;
R⁶ and R⁷ may be combined to form —(CR³³R³⁴)—(CR³⁵R³⁶)_{g}—(CR³⁷R³⁸)—, or —(CR³⁹R⁴⁰)ₕ—Z³— (CR⁴¹R⁴²)ᵢ—;
R⁸ and R⁹ may be combined to form —(CR⁴³R⁴⁴)—(CR⁴⁵R⁴⁶)ⱼ—(CR⁴⁷R⁴⁸)—, or —(CR⁴⁹R⁵⁰)ₖ—Z⁴— (CR⁵¹R⁵²)ᵣ—;
a, d, g, and j each independently represent 0, 1, 2, or 3;
b, c, e, f, h, i, k, and r each independently represent 1 or 2;
Z¹, Z², Z³, and Z⁴ are identical to or different from each other, and each represent an oxygen atom, a sulfur atom, or NR⁵³;
R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², and R⁵³ are identical to or different from each other, and each represent a C1-C4 alkyl group optionally having one or more halogen atoms, or a hydrogen atom;
when a, d, g, or j is 2 or 3, R¹⁵, R¹⁶, R²⁵, R²⁶, R³⁵, R³⁶, R⁴⁵, and R⁴⁶ of 2 or 3 may be each independently identical to or different from each other;
when b, c, e, f, h, i, k, or r is 2, R¹⁹, R²⁰, R²¹, R²², R²⁹, R³⁰, R³¹, R³², R³⁹, R⁴⁰, R⁴¹, R⁴², R⁴⁹, R⁵⁰, R⁵¹, and R⁵² of 2 may be each independently identical to or different from each other;
a plurality of R⁵³ (s) may be each independently identical to or different from each other;
R¹⁰ represents a C1-C3 chain hydrocarbon group optionally having one or more substituents selected from Group D, a C1-C4 alkoxycarbonyl group optionally having one or more halogen atoms, a cyano group, a formyl group, a carboxy group, a carbamoyl group, a halogen atom, or a hydrogen atom;
Y represents a single bond, an oxygen atom, or —S(O)ᵤ—;
when Y is a single bond, p represents 0, and Q represents a C1-C8 chain hydrocarbon group optionally having one or more substituents selected from Group F, a C3-C8 alicyclic hydrocarbon group optionally having one or more substituents selected from Group H, or a hydrogen atom;
when Y is an oxygen atom or —S(O)ᵤ—, p represents 0, 1, 2, 3, 4, 5, 6, or 7, and Q represents a C1-C8 chain hydrocarbon group optionally having one or more substituents selected from Group H, or a group selected from Group G;
u represents 0, 1, or 2;
R¹¹ and R¹² are identical to or different from each other, and each represent a C1-C4 alkyl group optionally having one or more halogen atoms, a halogen atom, or a hydrogen atom; and
when p is 1, 2, 3, 4, 5, 6, or 7, R¹¹ and R¹² of 1, 2, 3, 4, 5, 6, or 7 may be each independently identical to or different from each other;
Group A: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a phenyl group, a pyridyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a pyridazinyl group, a pyrimidinyl group, a pyazinyl group {the phenyl group, the pyridyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the pyridazinyl group, the pyrimidinyl group, and the pyrazinyl group may have one or more substituents selected from Group B}, a halogen atom, a hydroxy group, a cyano group, and CONR⁵⁴R⁵⁵;
R⁵⁴ and R⁵⁵ are identical to or different from each other, and each represent a C1-C4 alkyl group optionally having one or more halogen atoms or a hydrogen atom;
Group B: a group consisting of a C1-C4 chain hydrocarbon group optionally having one or more halogen atoms, a C1-C4 alkoxy group optionally having one or more halogen atoms, a C1-C4 alkylthio group optionally having one or more halogen atoms, a C1-C4 alkylsulfinyl group optionally having one or more halogen atoms, a C1-C4 alkylsulfonyl group optionally having one or more halogen atoms, a C1-C4 alkoxycarbonyl group optionally having one or more halogen atoms, a phenyl group optionally having one or more halogen atoms, a phenoxy group optionally having one or more halogen atoms, a cyano group, a nitro group, a carboxy group, a hydroxy group, a halogen atom, and CONR⁵⁴R⁵⁵;
Group C: a group consisting of a C1-C4 chain hydrocarbon group optionally having one or more halogen atoms, a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a phenyl group, a pyridyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a pyridazinyl group, a pyrimidinyl group, pyrazinyl group {the phenyl group, the pyridyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the pyridazinyl group, the pyrimidinyl group, and the pyrazinyl group may have one or more substituents selected from Group B}, a halogen atom, a hydroxy group, a cyano group, and CONR⁵⁴R⁵⁵;
Group D: a group consisting of a hydroxy group and a halogen atom;
Group E: a group consisting of a C1-C4 alkoxy group optionally having one or more halogen atoms, a cyano group, a hydroxy group, and a halogen atom;
Group F: a group consisting of a C3-C8 alicyclic hydrocarbon group, an indanyl group, a 1,2,3,4-tetrahydronaphthyl group, a phenyl group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a benzofuranyl group, a benzothienyl group, a 1,3-benzodioxolyl group, a 1,4-benzodioxanyl group {the C3-C8 alicyclic hydrocarbon group, the indanyl group, the 1,2,3,4-tetrahydronaphthyl group, the phenyl group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, the benzothienyl group, the 1,3-benzodioxolyl group, and the 1,4-benzodioxanyl group may have one or more substituents selected from Group C}, a C1-C4 alkoxycarbonyl group optionally having one or more halogen atoms, a halogen atom, a cyano group, a nitro group, a carboxyl group, a hydroxyl group, and CONR⁵⁴R⁵⁵;
Group G: a group consisting of a C3-C8 alicyclic hydrocarbon group, an indanyl group, a 1,2,3,4-tetrahydronaphthyl group, a phenyl group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a benzofuranyl group, and a benzothienyl group {the C3-C8 alicyclic hydrocarbon group, the indanyl group, the 1,2,3,4-tetrahydronaphthyl group, the phenyl group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, and the benzothienyl group may have one or more substituents selected from Group A},
Group H: a group consisting of a C3-C8 alicyclic hydrocarbon group, an indanyl group, a 1,2,3,4-tetrahydronaphthyl group, a phenyl group, a phenoxy group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a benzofuranyl group, a benzothienyl group, a 1,3-benzodioxolyl group, a 1,4-benzodioxanyl group {the C3-C8 alicyclic hydrocarbon group, the indanyl group, the 1,2,3,4-tetrahydronaphthyl group, the phenyl group, the phenoxy group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, the benzothienyl group, the 1,3-benzodioxolyl group, and the 1,4-benzodioxanyl group may have one or more substituents selected from Group C}, a C1-C4 alkoxycarbonyl group optionally having one or more halogen atoms, a halogen atom, a cyano group, a nitro group, a carboxyl group, a hydroxyl group, and CONR⁵⁴R⁵⁵](hereinafter, referred to as “Compound N of the present invention) or a salt thereof (hereinafter, the Compound N of the present invention or a salt thereof is referred to as “Compound of the present invention”).

[2] The compound or a salt thereof according to [1], wherein
R¹ represents a C1-C6 chain hydrocarbon group optionally having one or more substituents selected from Group A, a C3-C6 alicyclic hydrocarbon group optionally having one or more substituents selected from Group A, or a hydrogen atom.

[3] The compound or a salt thereof according to [1] or [2], wherein
R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, or a hydrogen atom.

[4] The compound or a salt thereof according to any one of [1] to [3], wherein
R¹⁰ represents a C1-C3 chain hydrocarbon group optionally having one or more substituents selected from Group D or a hydrogen atom.

[5] The compound or a salt thereof according to any one of [1] to [4], wherein
n represents 1.

[6] The compound or a salt thereof according to any one of [1] to [5], wherein
m represents 0 or 1.

[7] The compound or a salt thereof according to any one of [1] to [5], wherein
m represents 0.

[8]A composition for controlling a harmful arthropod comprising the compound or a salt thereof according to any one of [1] to [7], and an inert carrier.

[9]A composition comprising one or more ingredients selected from the group consisting of Group (a), Group (b), Group (c), and Group (d), and the compound or a salt thereof according to any one of [1] to [7]:
Group (a): a group consisting of insecticidal active ingredients, miticidal active ingredients, and nematicidal active ingredients;
Group (b): fungicidal active ingredients;
Group (c): plant growth modulating ingredients; and
Group (d): repellent ingredients.

[10]A method for controlling a harmful arthropod, which comprises applying an effective amount of the compound or a salt thereof according to any one of [1] to [7], or an effective amount of the composition according to [9] to a harmful arthropod or a habitat where the harmful arthropod lives.

[11]A seed or vegetative reproductive organ carrying an effective amount of the compound or a salt thereof according to any one of [1] to [7], or an effective amount of a composition according to [9].

### Effect of the Invention

The present invention can control harmful arthropods.

### MODE FOR CARRYING OUT THE INVENTION

The substituent in the present invention will be described.

The halogen atom means a fluorine atom, a chlorine atom, a bromine atom, or an iodine atom.

When the substituent has two or more halogen atoms or substituents, the halogen atoms or substituents may be identical to or different from each other.

The notation of “CX-CY” in the present description means that the number of carbon atoms is X to Y. For example, the notation of “C1-C6” means that the number of carbon atoms is 1 to 6.

The chain hydrocarbon group represents an alkyl group, an alkenyl group, or an alkynyl group.

Examples of the alkyl group include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, and a hexyl group.

Examples of the alkenyl group include a vinyl group, a 1-propenyl group, a 2-propenyl group, a 1-methyl-1-propenyl group, a 1-methyl-2-propenyl group, a 1,2-dimethyl-1-propenyl group, a 1-ethyl-2-propenyl group, a 3-butenyl group, a 4-pentenyl group, and a 5-hexenyl group.

Examples of the alkynyl group include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, and a 5-hexynyl group.

Examples of the alkoxy group include a methoxy group, an ethoxy group, a propoxy group, an isopropoxy group, a butoxy group, a tert-butoxy group, a pentyloxy group, and a hexyloxy group.

Examples of the alkylcarbonyl group include an acetyl group, a propanoyl group, a 2-methylpropanoyl group, and a hexanoyl group. More specifically, examples of the C1-C3 alkylcarbonyl group include an acetyl group, a propanoyl group, a butanoyl group, and an isobutanoyl group.

Examples of the alkoxycarbonyl group include a methoxycarbonyl group, an ethoxycarbonyl group, an isopropoxycarbonyl group, and a pentyloxycarbonyl group. More specifically, examples of the C1-C3 alkoxycarbonyl group include a methoxycarbonyl group, an ethoxycarbonyl group, a propoxycarbonyl group, and an isopropoxycarbonyl group.

Examples of the alkylsulfonyl group include a methylsulfonyl group, an ethylsulfonyl group, a propylsulfonyl group, and an isopropylsulfonyl group.

The alicyclic hydrocarbon group represents a cycloalkyl group, a cycloalkenyl group, or a cycloalkynyl group.

Examples of the cycloalkyl group include a cyclopropyl group, a cyclobutyl group, a cyclopentyl group, and a cyclohexyl group.

Examples of the cycloalkenyl group include a cyclopropenyl group, a cyclobutenyl group, a cyclopentenyl group, and a cyclohexenyl group.

Examples of the cycloalkynyl group include a cyclohexynyl group.

The Compound of the present invention may have one or more stereoisomers. Stereoisomers include enantiomers, diastereomers, geometric isomers, and the like. The Compound of the present invention includes each stereoisomer and a stereoisomer mixture in any ratio.

The Compound N of the present invention may form an acid addition salt. Examples of the acid forming the acid addition salt include inorganic acids such as hydrogen chloride, phosphoric acid, and sulfuric acid, and organic acids such as acetic acid, trifluoroacetic acid, benzoic acid, and p-toluenesulfonic acid. The acid addition salt is obtained, for example, by mixing the Compound N of the present invention and an acid.

Examples of an embodiment of the Compound N of the present invention include the following compounds.

[Embodiment 1] The Compound N of the present invention, wherein R⁴ and R⁵ are identical to or different from each other, and each represent a C1-C6 chain hydrocarbon group optionally having one or more substituents selected from Group A or a C3-C6 alicyclic hydrocarbon group optionally having one or more substituents selected from Group A, or R⁴ and R⁵ are combined to form —(CR²³R²⁴)—(CR²⁵R²⁶)_{d}— (CR²⁷R²⁰)— or —(CR²⁹R³⁰)ₑ—Z²— (CR³¹R³²)_{f}—.

[Embodiment 2] The Compound N of the present invention, wherein R⁴ and R⁵ are identical to or different from each other, and each represent a C1-C6 chain hydrocarbon group optionally having one or more substituents selected from Group A, or R⁴ and R⁵ are combined to form —(CR²³R²⁴)—(CR²⁵R²⁶)_{d}— (CR²⁷R²⁸)—.

[Embodiment 3] The Compound N of the present invention, wherein R⁴ and R⁵ are identical to or different from each other, and each represent a C1-C6 alkyl group, or R⁴ and R⁵ are combined to form —(CR²³R²⁴)—(CR²⁵R²⁶)_{d}— (CR²⁷R²⁸)—.

[Embodiment 4] The compound according to Embodiment 1, wherein R¹ represents a C1-C6 chain hydrocarbon group optionally having one or more substituents selected from Group A, a C3-C6 alicyclic hydrocarbon group optionally having one or more substituents selected from Group A, or a hydrogen atom.

[Embodiment 5] The compound according to Embodiment 2, wherein R¹ represents a C1-C6 chain hydrocarbon group optionally having one or more substituents selected from Group A, a C3-C6 alicyclic hydrocarbon group optionally having one or more substituents selected from Group A, or a hydrogen atom.

[Embodiment 6] The compound according to Embodiment 3, wherein R¹ represents a C1-C6 chain hydrocarbon group optionally having one or more substituents selected from Group A, a C3-C6 alicyclic hydrocarbon group optionally having one or more substituents selected from Group A, or a hydrogen atom.

[Embodiment 7] The compound according to Embodiment 4, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, a tetrahydrofuryl group, a tetrahydrothienyl group, {the tetrahydrofuryl group and the tetrahydrothienyl group may have one or more substituents selected from Group B}, or a hydrogen atom.

[Embodiment 8] The compound according to Embodiment 5, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, a tetrahydrofuryl group, a tetrahydrothienyl group, {the tetrahydrofuryl group and the tetrahydrothienyl group may have one or more substituents selected from Group B}, or a hydrogen atom.

[Embodiment 9] The compound according to Embodiment 6, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, a tetrahydrofuryl group, a tetrahydrothienyl group, {the tetrahydrofuryl group and the tetrahydrothienyl group may have one or more substituents selected from Group B}, or a hydrogen atom.

[Embodiment 10] The compound according to Embodiment 4, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, or a hydrogen atom.

[Embodiment 11] The compound according to Embodiment 5, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, or a hydrogen atom.

[Embodiment 12] The compound according to Embodiment 6, wherein R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, or a hydrogen atom.

[Embodiment 13] The compound according to Embodiment 7, wherein R¹⁰ represents a C1-C3 chain hydrocarbon group optionally having one or more substituents selected from Group D, a halogen atom, or a hydrogen atom.

[Embodiment 14] The compound according to Embodiment 8, wherein R¹⁰ represents a C1-C3 chain hydrocarbon group optionally having one or more substituents selected from Group D, a halogen atom, or a hydrogen atom.

[Embodiment 15] The compound according to Embodiment 9, wherein R¹⁰ represents a C1-C3 chain hydrocarbon group optionally having one or more substituents selected from Group D, a halogen atom, or a hydrogen atom.

[Embodiment 16] The compound according to Embodiment 10, wherein R¹⁰ represents a C1-C3 chain hydrocarbon group optionally having one or more substituents selected from Group D, a halogen atom, or a hydrogen atom.

[Embodiment 17] The compound according to Embodiment 11, wherein R¹⁰ represents a C1-C3 chain hydrocarbon group optionally having one or more substituents selected from Group D, a halogen atom, or a hydrogen atom.

[Embodiment 18] The compound according to Embodiment 12, wherein R¹⁰ represents a C1-C3 chain hydrocarbon group optionally having one or more substituents selected from Group D, a halogen atom, or a hydrogen atom.

[Embodiment 19] The compound according to Embodiment 7, wherein R¹⁰ represents a hydrogen atom.

[Embodiment 20] The compound according to Embodiment 8, wherein R¹⁰ represents a hydrogen atom.

[Embodiment 21] The compound according to Embodiment 9, wherein R¹⁰ represents a hydrogen atom.

[Embodiment 22] The compound according to Embodiment 10, wherein R¹⁰ represents a hydrogen atom.

[Embodiment 23] The compound according to Embodiment 11, wherein R¹⁰ represents a hydrogen atom.

[Embodiment 24] The compound according to Embodiment 12, wherein R¹⁰ represents a hydrogen atom.

[Embodiment 25] The compound according to Embodiment 13, wherein n represents 1.

[Embodiment 26] The compound according to Embodiment 14, wherein n represents 1.

[Embodiment 27] The compound according to Embodiment 15, wherein n represents 1.

[Embodiment 28] The compound according to Embodiment 16, wherein n represents 1.

[Embodiment 29] The compound according to Embodiment 17, wherein n represents 1.

[Embodiment 30] The compound according to Embodiment 18, wherein n represents 1.

[Embodiment 31] The compound according to Embodiment 19, wherein n represents 1.

[Embodiment 32] The compound according to Embodiment 20, wherein n represents 1.

[Embodiment 33] The compound according to Embodiment 21, wherein n represents 1.

[Embodiment 34] The compound according to Embodiment 22, wherein n represents 1.

[Embodiment 35] The compound according to Embodiment 23, wherein n represents 1.

[Embodiment 36] The compound according to Embodiment 24, wherein n represents 1.

[Embodiment 37] The compound according to Embodiment 25, wherein m represents 0 or 1.

[Embodiment 38] The compound according to Embodiment 26, wherein m represents 0 or 1.

[Embodiment 39] The compound according to Embodiment 27, wherein m represents 0 or 1.

[Embodiment 40] The compound according to Embodiment 28, wherein m represents 0 or 1.

[Embodiment 41] The compound according to Embodiment 29, wherein m represents 0 or 1.

[Embodiment 42] The compound according to Embodiment 30, wherein m represents 0 or 1.

[Embodiment 43] The compound according to Embodiment 31, wherein m represents 0 or 1.

[Embodiment 44] The compound according to Embodiment 32, wherein m represents 0 or 1.

[Embodiment 45] The compound according to Embodiment 33, wherein m represents 0 or 1.

[Embodiment 46] The compound according to Embodiment 34, wherein m represents 0 or 1.

[Embodiment 47] The compound according to Embodiment 35, wherein m represents 0 or 1.

[Embodiment 48] The compound according to Embodiment 36, wherein m represents 0 or 1.

[Embodiment 49] The compound according to Embodiment 25, wherein m represents 0.

[Embodiment 50] The compound according to Embodiment 26, wherein m represents 0.

[Embodiment 51] The compound according to Embodiment 27, wherein m represents 0.

[Embodiment 52] The compound according to Embodiment 28, wherein m represents 0.

[Embodiment 53] The compound according to Embodiment 29, wherein m represents 0.

[Embodiment 54] The compound according to Embodiment 30, wherein m represents 0.

[Embodiment 55] The compound according to Embodiment 31, wherein m represents 0.

[Embodiment 56] The compound according to Embodiment 32, wherein m represents 0.

[Embodiment 57] The compound according to Embodiment 33, wherein m represents 0.

[Embodiment 58] The compound according to Embodiment 34, wherein m represents 0.

[Embodiment 59] The compound according to Embodiment 35, wherein m represents 0.

[Embodiment 60] The compound according to Embodiment 36, wherein m represents 0.

[Embodiment 61] The compound according to any one of Embodiment 1 to Embodiment 60 or the Compound N of the present invention, wherein Y represents a single bond, and Q represents a C1-C8 chain hydrocarbon group optionally having one or more substituents selected from Group F or a C3-C8 alicyclic hydrocarbon group optionally having one or more substituents selected from Group H.

[Embodiment 62] The compound according to any one of Embodiment 1 to Embodiment 60 or the Compound N of the present invention, wherein Y represents a single bond, and Q represents a C1-C8 chain hydrocarbon group optionally having one or more substituents selected from Group F.

[Embodiment 63] The compound according to any one of Embodiment 1 to Embodiment 60 or the Compound N of the present invention, wherein Y represents an oxygen atom, p represents 1, and Q represents a C1-C8 chain hydrocarbon group optionally having one or more substituents selected from Group H or a group selected from Group G.

[Embodiment A1] The Compound N of the present invention, wherein R¹⁰ represents a C1-C3 chain hydrocarbon group optionally having one or more substituents selected from Group D, a halogen atom, or a hydrogen atom.

[Embodiment A2] The Compound N of the present invention, wherein R¹⁰ represents a methyl group, a halogen atom, or a hydrogen atom.

[Embodiment A3] The Compound N of the present invention, wherein R¹⁰ represents a hydrogen atom.

[Embodiment A4] The compound according to Embodiment A1, wherein Y represents a single bond, and Q represents a C1-C8 chain hydrocarbon group optionally having one or more substituents selected from Group F or a C3-C8 alicyclic hydrocarbon group optionally having one or more substituents selected from Group H.

[Embodiment A5] The compound according to Embodiment A2, wherein Y represents a single bond, and Q represents a C1-C8 chain hydrocarbon group optionally having one or more substituents selected from Group F or a C3-C8 alicyclic hydrocarbon group optionally having one or more substituents selected from Group H.

[Embodiment A6] The compound according to Embodiment A3, wherein Y represents a single bond, and Q represents a C1-C8 chain hydrocarbon group optionally having one or more substituents selected from Group F or a C3-C8 alicyclic hydrocarbon group optionally having one or more substituents selected from Group H.

[Embodiment A7] The compound according to Embodiment A1, wherein Y represents a single bond, and Q represents a C1-C8 chain hydrocarbon group optionally having one or more substituents selected from Group F.

[Embodiment A8] The compound according to Embodiment A2, wherein Y represents a single bond, and Q represents a C1-C8 chain hydrocarbon group optionally having one or more substituents selected from Group F.

[Embodiment A9] The compound according to Embodiment A3, wherein Y represents a single bond, and Q represents a C1-C8 chain hydrocarbon group optionally having one or more substituents selected from Group F.

[Embodiment A10] The compound according to Embodiment A1, wherein Y represents a single bond, and Q represents a C1-C8 chain hydrocarbon group optionally having one or more substituents selected from Group F²,
Group F²: a group consisting of a C3-C6 alicyclic hydrocarbon group, a phenyl group, a naphthyl group, a benzofuranyl group, a benzothienyl group {the C3-C6 alicyclic hydrocarbon group, the phenyl group, the naphthyl group, the benzofuranyl group, and the benzothienyl group may have one or more substituents selected from Group C²}, and a halogen atom,
Group C²: a group consisting of a C1-C3 alkyl group optionally having one or more halogen atoms, a C1-C3 alkoxy group optionally having one or more halogen atoms, a phenyl group optionally having one or more substituents selected from Group B², and a halogen atom, and
Group B²: a group consisting of a C1-C3 alkyl group optionally having one or more halogen atoms, a C1-C3 alkoxy group optionally having one or more halogen atoms, and a halogen atom.

[Embodiment A11] The compound according to Embodiment A2, wherein Y represents a single bond, and Q represents a C1-C8 chain hydrocarbon group optionally having one or more substituents selected from Group F².

[Embodiment A12] The compound according to Embodiment A3, wherein Y represents a single bond, and Q represents a C1-C8 chain hydrocarbon group optionally having one or more substituents selected from Group F².

[Embodiment A13] The compound according to Embodiment A1, wherein Y represents an oxygen atom, p represents 1, and Q represents a C1-C8 chain hydrocarbon group optionally having one or more substituents selected from Group H or a group selected from Group G.

[Embodiment A14] The compound according to Embodiment A2, wherein Y represents an oxygen atom, p represents 1, and Q represents a C1-C8 chain hydrocarbon group optionally having one or more substituents selected from Group H or a group selected from Group G.

[Embodiment A15] The compound according to Embodiment A3, wherein Y represents an oxygen atom, p represents 1, and Q represents a C1-C8 chain hydrocarbon group optionally having one or more substituents selected from Group H or a group selected from Group G.

[Embodiment A16] The compound according to Embodiment A1, wherein Y represents an oxygen atom, p represents 1, R¹¹ and R¹² each represent a hydrogen atom, and Q represents a C1-C8 chain hydrocarbon group optionally having one or more substituents selected from Group H² or a group selected from Group G²,
Group H²: a group consisting of a C3-C6 alicyclic hydrocarbon group, a phenyl group, a naphthyl group, a benzofuranyl group, a benzothienyl group {the C3-C6 alicyclic hydrocarbon group, the phenyl group, the naphthyl group, the benzofuranyl group, and the benzothienyl group may have one or more substituents selected from Group C²}, and a halogen atom,
Group G²: a group consisting of a C3-C6 alicyclic hydrocarbon group, a phenyl group, a naphthyl group, a benzofuranyl group, a benzothienyl group {the C3-C6 alicyclic hydrocarbon group, the phenyl group, the naphthyl group, the benzofuranyl group, and the benzothienyl group may have one or more substituents selected from Group A²}, and a halogen atom, and
Group A²: a group consisting of a C1-C3 alkoxy group optionally having one or more halogen atoms and a phenyl group optionally having one or more halogen atoms.

[Embodiment A17] The compound according to Embodiment A2, wherein Y represents an oxygen atom, p represents 1, R¹¹ and R¹² each represent a hydrogen atom, and Q represents a C1-C8 chain hydrocarbon group optionally having one or more substituents selected from Group H² or a group selected from Group G².

[Embodiment A18] The compound according to Embodiment A3, wherein Y represents an oxygen atom, p represents 1, R¹¹ and R¹² each represent a hydrogen atom, and Q represents a C1-C8 chain hydrocarbon group optionally having one or more substituents selected from Group H² or a group selected from Group G².

[Embodiment B1] The Compound N of the present invention, wherein R⁴ represents a C1-C3 alkyl group optionally having one or more halogen atoms, and R⁵ represents a C1-C3 alkyl group optionally having one or more halogen atoms or a hydrogen atom, or R⁴ and R⁵ are combined to form —(CH₂)₄—.

[Embodiment B2] The Compound N of the present invention, wherein R⁴ and R⁵ are identical to or different from each other, and each represent a C1-C3 alkyl group optionally having one or more halogen atoms, or R⁴ and R⁵ are combined to form —(CH₂)₄—.

[Embodiment B3] The Compound N of the present invention, wherein R⁴ and R⁵ are identical to or different from each other, and each represent a C1-C3 alkyl group, or R⁴ and R⁵ are combined to form —(CH₂)₄—.

[Embodiment B4] The Compound N of the present invention, R⁴ and R⁵ are combined to form —(CH₂)₄—.

[Embodiment B5] The compound according to Embodiment B1, wherein R¹ represents a C1-C4 alkyl group optionally having one or more halogen atoms or a hydrogen atom.

[Embodiment B6] The compound according to Embodiment B2, wherein R¹ represents a C1-C4 alkyl group optionally having one or more halogen atoms or a hydrogen atom.

[Embodiment B7] The compound according to Embodiment B3, wherein R¹ represents a C1-C4 alkyl group optionally having one or more halogen atoms or a hydrogen atom.

[Embodiment B8] The compound according to Embodiment B4, wherein R¹ represents a C1-C4 alkyl group optionally having one or more halogen atoms or a hydrogen atom.

[Embodiment B9] The compound according to Embodiment B5, wherein R⁸ and R⁹ are identical to or different from each other, and each represent a C1-C20 chain hydrocarbon group optionally having one or more substituents selected from Group A², a C3-C6 cycloalkyl group optionally having one or more substituents selected from Group A², or a hydrogen atom.

[Embodiment B10] The compound according to Embodiment B5, wherein R⁸ and R⁹ are identical to or different from each other, and each represent a C1-C20 chain hydrocarbon group optionally having one or more substituents selected from Group A² or a hydrogen atom.

[Embodiment B11] The compound according to Embodiment B6, wherein R⁸ and R⁹ are identical to or different from each other, and each represent a C1-C20 chain hydrocarbon group optionally having one or more substituents selected from Group A², a C3-C6 cycloalkyl group optionally having one or more substituents selected from Group A², or a hydrogen atom.

[Embodiment B12] The compound according to Embodiment B6, wherein R⁸ and R⁹ are identical to or different from each other, and each represent a C1-C20 chain hydrocarbon group optionally having one or more substituents selected from Group A² or a hydrogen atom.

[Embodiment B13] The compound according to Embodiment B7, wherein R⁸ and R⁹ are identical to or different from each other, and each represent a C1-C20 chain hydrocarbon group optionally having one or more substituents selected from Group A², a C3-C6 cycloalkyl group optionally having one or more substituents selected from Group A², or a hydrogen atom.

[Embodiment B14] The compound according to Embodiment B7, wherein R⁸ and R⁹ are identical to or different from each other, and each represent a C1-C20 chain hydrocarbon group optionally having one or more substituents selected from Group A² or a hydrogen atom.

[Embodiment B15] The compound according to Embodiment B8, wherein R⁸ and R⁹ are identical to or different from each other, and each represent a C1-C20 chain hydrocarbon group optionally having one or more substituents selected from Group A², a C3-C6 cycloalkyl group optionally having one or more substituents selected from Group A², or a hydrogen atom.

[Embodiment B16] The compound according to Embodiment B8, wherein R⁸ and R⁹ are identical to or different from each other, and each represent a C1-C20 chain hydrocarbon group optionally having one or more substituents selected from Group A² or a hydrogen atom.

[Embodiment B17] The compound according to any one of Embodiment B1 to Embodiment B16 or Compound N of the present invention, wherein n represents 1, and R⁶ and R⁷ each represent a hydrogen atom.

[Embodiment B18] The compound according to any one of Embodiment B1 to Embodiment B16 or Compound N of the present invention, wherein n represents 1, R⁶ and R⁷ each represent a hydrogen atom, m represents 0 or 1, and R² and R³ each represent a hydrogen atom.

[Embodiment B19] The compound according to any one of Embodiment B1 to Embodiment B16 or Compound N of the present invention, wherein n represents 1, R⁶ and R⁷ each represent a hydrogen atom, and m represents 0.

A compound according to any one of Embodiments A1 to A18 and any one of Embodiments B1 to B19.

Then, a method for producing the present invention compound will be described.

### Production Method 1

A compound represented by formula (I) (that is, the Compound N of the present invention) can be produced by reacting a compound represented by formula (M-1) (hereinafter, referred to as a compound (M-1)) with a compound represented by formula (M-2) (hereinafter, referred to as a compound (M-2)), in the presence of a condensing agent.

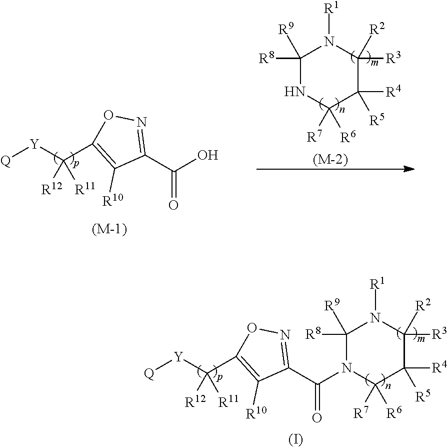

[In the formula, symbols represent the same meaning as described above.]

The reaction is typically performed in a solvent. Examples of the solvent used in the reaction include aromatic hydrocarbons such as benzene and toluene (hereinafter, referred to as aromatic hydrocarbons); aliphatic hydrocarbons such as hexane (hereinafter, referred to as aliphatic hydrocarbons); ethers such as diethyl ether and tetrahydrofuran (hereinafter, referred to as THF) (hereinafter, referred to as ethers); halogenated hydrocarbons such as dichloromethane, chloroform, 1,2-dichloroethane, and chlorobenzene (hereinafter, referred to as halogenated hydrocarbons); acid amides such as dimethylformamide (hereinafter, referred to as DMF) (hereinafter, referred to as acid amides); esters such as ethyl acetate and butyl acetate (hereinafter, referred to as esters); and mixtures thereof.

Examples of the condensing agent used in the reaction include 1-ethyl-3-(3-dimethylaminopropyl) carbodiimide hydrochloride, dicyclohexylcarbodiimide, benzotriazole-1-yloxytris (dimethylamino)phosphonium hexafluorophosphate, and (benzotriazole-1-yloxy)tripyrrolidinophosphonium hexafluorophosphate. The amount of the condensing agent used in the reaction can be typically used in an arbitrary ratio of 1 mol to an excess amount, and is preferably 1 mol to 3 mol, with respect to 1 mol of the compound (M-1).

The reaction may be performed in the presence of a base as necessary. Examples of the base used in the reaction include: carbonates such as sodium carbonate and potassium carbonate (hereinafter, referred to as a carbonate); tertiary amines such as triethylamine, diisopropylethylamine, 1,8-diazabicyclo [5.4.0]undec-7-ene and 1,5-diazabicyclo [4.3.0]non-5-ene (hereinafter, referred to as tertiary amines); and nitrogen-containing aromatic compounds such as pyridine and 4-dimethylaminopyridine (hereinafter, referred to as a nitrogen-containing aromatic compound). The amount of the base used in the reaction can be typically used in an arbitrary ratio of 1 mol to an excess amount, and is preferably 1 mol to 3 mol, with respect to 1 mol of the compound (M-1).

The reaction may be performed in the presence of 1-hydroxybenzotriazole, 1-hydroxy-7-azabenzotriazole, N-hydroxysuccinimide, or the like in place of or in addition to the base, as necessary. These amounts are typically used in an arbitrary ratio of 0.01 mol to 1 mol, and preferably in a ratio of 0.05 mol to 0.2 mol, with respect to 1 mol of the compound (M-1).

The reaction time of the reaction is typically in the range of 5 minutes to 72 hours. The reaction temperature of the reaction is typically in the range of −20° C. to 100° C. (however, when the boiling point of the solvent to be used is lower than 100° C., the temperature is −20° C. to the boiling point of the solvent).

In the reaction, the molar ratio of the compound (M-1) to the compound (M-2) used can be arbitrarily set, but is preferably equimolar or a ratio close thereto, for example, a ratio of the compound (M-2) to 1 mol of the compound (M-1) is from 1 mol to 3 mol.

After completion of the reaction, water is added to the reaction mixture, the mixture is extracted with an organic solvent, and the organic layer is subjected to post-treatment operations such as drying and concentration, thereby allowing to provide the Compound N of the present invention.

The compound (M-2) is a commercially available compound, or can be produced according to a known method.

### Production Method 2

The Compound N of the present invention can also be produced by reacting a compound represented by formula (M-3) (hereinafter, referred to as a compound (M-3)) with the compound (M-2), in the presence of a base.

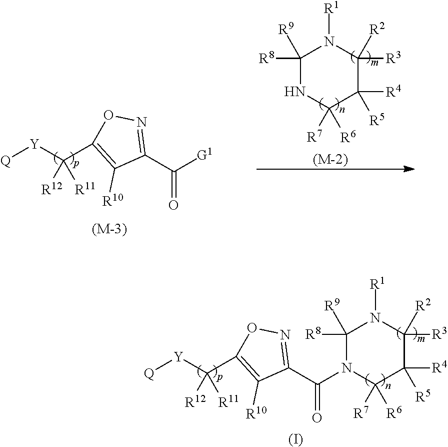

[In the formula, G1 represents a chlorine atom, a bromine atom, or an iodine atom, and other symbols represent the same meaning as described above.]

The reaction is typically performed in a solvent. Examples of the solvent used in the reaction include: ethers; aliphatic hydrocarbons; aromatic hydrocarbons; halogenated hydrocarbons; esters; nitriles such as acetonitrile and butyronitrile (hereinafter, referred to as nitriles); acid amide; sulfoxides such as dimethyl sulfoxide (hereinafter, referred to as DMSO) (hereinafter, referred to as sulfoxides); and mixtures thereof.

Examples of the base used in the reaction include carbonates, tertiary amines, and nitrogen-containing aromatic compounds. The amount of the base used in the reaction can be typically used in an arbitrary ratio of 1 mol to an excess amount, and is preferably 1 mol to 3 mol, with respect to 1 mol of the compound (M-3).

The reaction time of the reaction is typically in the range of 5 minutes to 72 hours. The reaction temperature of the reaction is typically in the range of −20° C. to 100° C.

In the reaction, the molar ratio of the compound (M-2) to the compound (M-3) used can be arbitrarily set, but is preferably equimolar or a ratio close thereto, specifically, from 0.5 mol to 3 mol of the compound (M-2) with respect to 1 mol of the compound (M-3).

After completion of the reaction, water is added to the reaction mixture, the mixture is extracted with an organic solvent, and the organic layer is subjected to post-treatment operations such as drying and concentration, thereby allowing to provide the Compound N of the present invention.

### Production Method 3

The Compound N of the present invention can also be produced by reacting a compound represented by formula (M-4) (hereinafter, referred to as a compound (M-4)) with the compound (M-2).

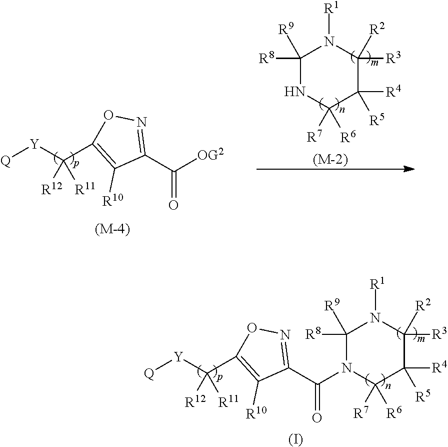

[In the formula, G² represents a methyl group or an ethyl group, and other symbols represent the same meaning as described above.]

The reaction is performed in a solvent or in the absence of a solvent. Examples of the solvent used in the reaction include ethers, aliphatic hydrocarbons, aromatic hydrocarbons, halogenated hydrocarbons, esters, nitriles, acid amides, sulfoxides, and mixtures thereof.

The reaction time of the reaction is typically in the range of 5 minutes to 72 hours. The reaction temperature of the reaction is typically in the range of 50° C. to 200° C. In the reaction, the molar ratio of the compound (M-2) to the compound (M-4) used can be arbitrarily set, but is preferably equimolar or a ratio close thereto, specifically, from 0.5 mol to 3 mol of the compound (M-2) with respect to 1 mol of the compound (M-4).

After completion of the reaction, water is added to the reaction mixture, the mixture is extracted with an organic solvent, and the organic layer is subjected to post-treatment operations such as drying and concentration, thereby allowing to provide the Compound N of the present invention.

### Production Method 4

The Compound N of the present invention can also be produced by reacting a compound represented by formula (M-5) (hereinafter, referred to as a compound (M-5)) with a compound represented by formula (M-6) (hereinafter, referred to as a compound (M-6)).

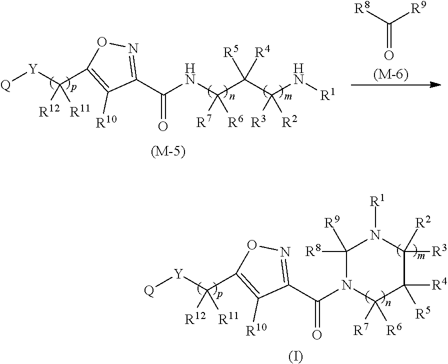

[In the formula, symbols represent the same meaning as described above.]

The reaction is performed in a solvent or in the absence of a solvent. Examples of the solvent used in the reaction include ethers, aliphatic hydrocarbons, aromatic hydrocarbons, halogenated hydrocarbons, esters, nitriles, acid amides, sulfoxides, and mixtures thereof.

The reaction time of the reaction is typically in the range of 5 minutes to 72 hours. The reaction temperature of the reaction is typically in the range of −20° C. to 160° C.

In the reaction, the molar ratio of the compound (M-6) to the compound (M-5) used can be arbitrarily set, but the compound (M-6) is preferably 1 mol to 50 mol, with respect to 1 mol of the compound (M-5).

After completion of the reaction, water is added to the reaction mixture, the mixture is extracted with an organic solvent, and the organic layer is subjected to post-treatment operations such as drying and concentration, thereby allowing to provide the Compound N of the present invention.

The compound (M-6) is a commercially available compound, or can be produced according to a known method. When R⁸ and R⁹ each represent a hydrogen atom in the compound (M-6), that is, when the compound (M-6) is formaldehyde, paraformaldehyde can be used instead of the compound (M-6).

Hereinafter, a method for producing a production intermediate will be described.

### Reference Production Example 1

The compound (M-1) can be produced by subjecting the compound (M-4) to a hydrolysis reaction in the presence of a base.

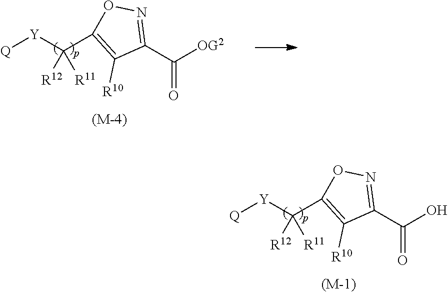

[In the formula, symbols represent the same meaning as described above.]

The reaction is performed in the presence of a base and in the presence of water and an organic solvent. Examples of the solvent used in the reaction include ethers; nitriles; alcohols such as methanol, ethanol, and propanol; and mixtures thereof.

Examples of the base used in the reaction include alkali metal hydroxides such as lithium hydroxide, sodium hydroxide, and potassium hydroxide.

The reaction time of the reaction is typically in the range of 5 minutes to 72 hours. The reaction temperature of the reaction is typically in the range of 0° C. to 100° C.

The amount of the base used in the reaction can be typically used in an arbitrary ratio of 1 mol to an excess amount, and is preferably 1 mol to 5 mol, with respect to 1 mol of the compound (M-1).

After completion of the reaction, water is added to the reaction mixture, the mixture is extracted with an organic solvent, and the organic layer is subjected to post-treatment operations such as drying and concentration, thereby allowing to provide the compound (M-1).

Reference production example 2 The compound (M-3) can be produced by reacting the compound (M-1) with a halogenating agent.

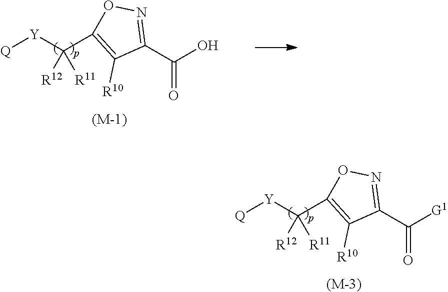

[In the formula, symbols represent the same meaning as described above.]

The reaction is typically performed in a solvent. Examples of the solvent used in the reaction include ethers, aliphatic hydrocarbons, aromatic hydrocarbons, halogenated hydrocarbons, and mixtures thereof.

Examples of the halogenating agent used in the reaction include thionyl chloride, oxalyl chloride, phosphorus oxychloride, phosphorus oxybromide, and phosphorus tribromide.

The reaction time of the reaction is typically in the range of 5 minutes to 24 hours. The reaction temperature is typically in the range of 0 to 100° C.

The amount of the halogenating agent used in the reaction can be typically used in an arbitrary ratio of 1 mol to an excess amount, and is preferably 1 mol to 5 mol, with respect to 1 mol of the compound (M-1).

After completion of the reaction, the reaction mixture is subjected to post-treatment operations such as concentration as it is, whereby the compound (M-3) can be obtained.

### Reference Production Example 3

The compound (M-4) can be produced by reacting a compound represented by formula (M-7) (hereinafter, referred to as a compound (M-7)) with a compound represented by formula (M-8) (hereinafter, referred to as a compound (M-8)).

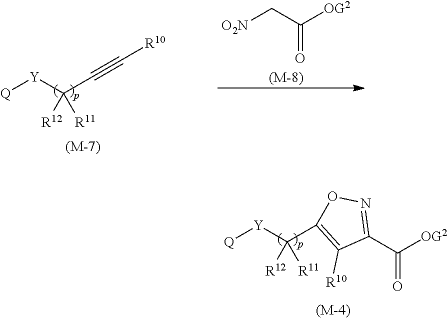

[In the formula, symbols represent the same meaning as described above.]

The reaction is typically performed in a solvent. Examples of the solvent used in the reaction include ethers, aliphatic hydrocarbons, aromatic hydrocarbons, halogenated hydrocarbons, and mixtures thereof.

The reaction time of the reaction is typically in the range of 5 minutes to 24 hours. The reaction temperature is typically in the range of 0 to 100° C.

In the reaction, the molar ratio of the compound (M-8) to the compound (M-7) used can be arbitrarily set, but the compound (M-8) is preferably 1 mol to 10 mol, with respect to 1 mol of the compound (M-7).

After completion of the reaction, water is added to the reaction mixture, the mixture is extracted with an organic solvent, and the organic layer is subjected to post-treatment operations such as drying and concentration, thereby allowing to provide the compound (M-4).

The compound (M-7) is a commercially available compound, or can be produced according to a known method.

The compound (M-8) is a commercially available compound.

### Reference Production Example 4

The compound (M-2) can be produced by reacting a compound represented by formula (M-9) (hereinafter, referred to as a compound (M-9)) with the compound (M-6).

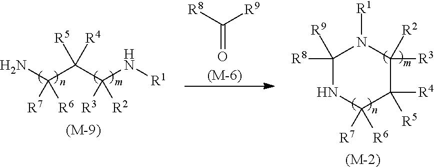

[In the formula, symbols represent the same meaning as described above.]

The reaction can be performed in accordance with Production method 4, using the compound (M-9) in place of the compound (M-5).

The compound (M-9) is a commercially available compound, or can be produced according to a known method.

Reference production example 5 The compound (M-5) can be produced by reacting the compound (M-1) with the compound (M-9), in the presence of a condensing agent.

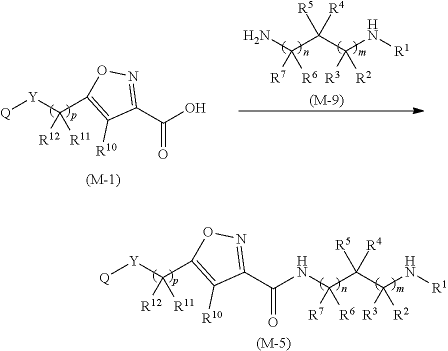

[In the formula, symbols represent the same meaning as described above.]

The reaction can be performed in accordance with Production method 1, using the compound (M-9) in place of the compound (M-2).

The Compound of the present invention may be mixed with or used in combination with one or more ingredient(s) selected from the group consisting of the following Group (a), Group (b), Group (c), and Group (d) (hereinafter referred to as “Present ingredient”).

When the Compound of the present invention is mixed with or used in combination with the Present ingredient, they are used simultaneously, separately, or at time intervals with each other.

When the Compound of the present invention is used simultaneously with the Present ingredient, the Compound of the present invention and the Present ingredient may be contained in separate formulations or contained in one formulation.

One aspect of the present invention provides a composition comprising one or more ingredient(s) selected from the group consisting of Group (a), Group (b), Group (c), and Group (d), and the Compound of the present invention (hereinafter referred to as “Composition A”).

Group (a) is a group consisting of acetylcholinesterase inhibitors (for example, carbamate insecticides and organophosphate insecticides), GABA-gated chloride channel blockers (for example, phenylpyrazole insecticides), sodium channel modulators (for example, pyrethroid insecticides), nicotinic acetylcholine receptor competitive modulators (for example, neonicotinoid insecticides), nicotinic acetylcholine receptor allosteric modulators, glutamate-gated chloride channel allosteric modulators (for example, macrolide insecticides), juvenile hormone mimics, multisite inhibitors, chordotonal organ TRPV channel modulators, mite growth inhibitors, microbial disruptors of insect midgut membranes, inhibitors of mitochondrial ATP synthase, uncouplers of oxidative phosphorylation, nicotinic acetylcholine receptor channel blockers (for example, nereistoxin insecticides), inhibitors of chitin biosynthesis, moulting disruptors, ecdysone receptor agonists, octopamine receptor agonists, mitochondrial complexes I, II, III, and IV electron transport inhibitors, voltage-dependent sodium channel blockers, inhibitors of acetyl CoA carboxylase, ryanodine receptor modulators (for example, diamide insecticides), chordotonal organ modulators, and microbial insecticides, and other insecticidal active ingredients, miticidal active ingredients, and nematicidal active ingredients. These ingredients are described in the classification on the basis of action mechanism by IRAC.

Group (b) is a group consisting of nucleic acids synthesis inhibitors (for example, phenylamide fungicides and acylamino acid fungicides), cell division and cytoskeleton inhibitors (for example, MBC fungicides), respiration inhibitors (for example, QoI fungicides, and QiI fungicides), amino acids synthesis and protein synthesis inhibitors (for example, anilino-pyrimidine fungicides), signal transduction inhibitors, lipid synthesis and membrane synthesis inhibitors, sterol biosynthesis inhibitors (for example, DMI fungicides such as triazole fungicides), cell wall biosynthesis inhibitors, melanin synthesis inhibitors, plant defense inducers, fungicides with multi-site contact activity, microbial fungicides, and other fungicidal active ingredients. These ingredients are described in the classification on the basis of action mechanism by FRAC.

Group (c) is a group of plant growth regulatory ingredients (including mycorrhizal fungi and root nodule bacteria).

Group (d) is a group of repellent ingredients.

Hereinafter, examples of the combination of the Present ingredient and the Compound of the present invention are described. For example, “alanycarb+SX” indicates a combination of alanycarb and SX.

The abbreviation of “SX” indicates any one of the Compound of the present invention selected from the Compound groups SX1 to SX210 or the Compounds of the present invention 1 to 108 described in Examples. Also, all of the following Present ingredient are known ingredients, and may be obtained from commercially available formulations, or may be prepared by known methods. When the Present ingredient is a microorganism, it may also be available from a bacterial authority depository. Further, the number in parentheses represents the CAS RN (registered trademark).

Combinations of the Present ingredient in the above Group (a) and the Compound of the present invention:
abamectin+SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+SX, aluminium phosphide+SX, amitraz+SX, azadirachtin+SX, azamethiphos+SX, azinphos-ethyl+SX, azinphos-methyl+SX, azocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cyclaniliprole+SX, cyclobutrifluram+SX, cycloprothrin+SX, cycloxaprid+SX, cyenopyrafen+SX, cyetpyrafen+SX, cyflumetofen+SX, cyfluthrin+SX, cyhalodiamide+SX, cyhalothrin+SX, cyhexatin+SX, cypermethrin+SX, cyphenothrin+SX, cyproflanilide+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC(2-methyl-4,6-dinitrophenol)+SX, doramectin+SX, dried leaves of Dryopteris filix-mas+SX, emamectin-benzoate+SX, empenthrin+SX, endosulfan+SX, EPN(O-ethyl O-(4-nitrophenyl)phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Azadirachta indica+SX, extract of Cassia nigricans+SX, extract of clitoria ternatea+SX, extract of Symphytum officinale+SX, extract of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenmezoditiaz+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, fluchlordiniliprole+SX, flucycloxuron+SX, flucythrinate+SX, fluensulfone+SX, flufenoprox+SX, flufenoxuron+SX, flufiprole+SX, flumethrin+SX, flupentiofenox+SX, flupyradifurone+SX, flupyrimin+SX, flupyroxystrobin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indazapyroxamet+SX, indoxacarb+SX, isocycloseram+SX, isofenphos+SX, isoprocarb+SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, ledprona+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, mivorilaner+SX, modoflaner+SX, momfluorothrin+SX, monocrotophos+SX, moxidectin+SX, naled+SX, nicofluprole+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spidoxamat+SX, spinetoram+SX, spinosad+SX, spirobudifen+SX, spirodiclofen+SX, spiromesifen+SX, spiropidion+SX, spirotetramat+SX, sulfiflumin+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tigolaner+SX, tiorantraniliprole+SX, tioxazafen+SX, tolfenpyrad+SX, tralomethrin+SX, transfluthrin+SX, triazamate+SX, triazophos+SX, trichlorfon+SX, trifluenfuronate+SX, triflumezopyrim+SX, triflumuron+SX, trimethacarb+SX, tyclopyrazoflor+SX, umifoxolaner+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, N-{4-chloro-3-[(1-cyanocyclopropyl)carbamoyl]phenyl}-1-methyl-4-(methanesulfonyl)-3-(1,1,2,2,2-pentafluoroethyl)-1H-pyrazole-3-carboxamide (1400768-21-9)+SX, N-[3-chloro-1-(pyridin-3-yl)-1H-pyrazol-4-yl]-2-(methanesulfonyl)propanamide (2396747-83-2)+SX, N-[4-chloro-2-(pyridin-3-yl)-1,3-thiazol-5-yl]-N-ethyl-3-(methanesulfonyl)propanamide+SX, 1,4-dimethyl-2-[2-(pyridin-3-yl)-2H-indazol-5-yl]-1,2,4-triazolidine-3,5-dione (2171099-09-3)+SX, 2-isopropyl-5-[(3,4,4-trifluoro-3-buten-1-yl)sulfonyl]-1,3,4-thiadiazole (2058052-95-0)+SX, N-({2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide+SX, 7-fluoro-N-[1-(methylsulfanyl)-2-methylpropan-2-yl]-2-(pyridin-3-yl)-2H-indazole-4-carboxamide+SX, 7-fluoro-N-[1-(methanesulfinyl)-2-methylpropan-2-yl]-2-(pyridin-3-yl)-2H-indazole-4-carboxamide+SX, 7-fluoro-N-[1-(methanesulfonyl)-2-methylpropan-2-yl]-2-(pyridin-3-yl)-2H-indazole-4-carboxamide+SX, N-[1-(difluoromethyl)cyclopropyl]-2-(pyridin-3-yl)-2H-indazole-4-carboxamide+SX, 2,9-dihydro-9-(methoxymethyl)-2-(pyridin-3-yl)-10H-pyrazolo[3,4-f]pyrido[2,3-b][1,4]oxazepin-10-one (2607927-97-7)+SX, BT crop protein CrylAb+SX, BT crop protein CrylAc+SX, BT crop protein CrylFa+SX, BT crop protein CrylA.105+SX, BT crop protein Cry2Ab+SX, BT crop protein Vip3A+SX, BT crop protein mCry3A+SX, BT crop protein Cry3Ab+SX, BT crop protein Cry3Bb+SX, BT crop protein Cry34Ab1/Cry35Ab1+SX, Adoxophyes orana granulosis virus strain BV-0001+SX, Anticarsia gemmatalis MNPV (multiple nucleocapsid nucleopolyhedrovirus)+SX, Autographa californica MNPV+SX, Cydia pomonella GV (granulovirus) strain V15+SX, Cydia pomonella GV strain V22+SX, Cryptophlebia leucotreta GV+SX, Dendrolimus punctatus cypovirus+SX, Helicoverpa armigera NPV (nucleopolyhedrovirus) strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua MNPV+SX, Spodoptera littoralis MNPV+SX, Spodoptera litura NPV+SX, Arthrobotrys dactyloides+SX, Bacillus firmus strain GB-126+SX, Bacillus firmus strain I-1582+SX, Bacillus firmus strain NCIM2637+SX, Bacillus megaterium+SX, Bacillus sp. strain AQ175+SX, Bacillus sp. strain AQ177+SX, Bacillus sp. strain AQ178+SX, Bacillus sphaericus strain 2362 serotype H5a5b+SX, Bacillus sphaericus strain ABTS1743+SX, Bacillus thuringiensis strain AQ52+SX, Bacillus thuringiensis strain BD #32+SX, Bacillus thuringiensis strain CR-371+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai strain NB200+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain CCT1306)+SX, Bacillus thuringiensis subsp. Kurstaki strain EG2348+SX, Bacillus thuringiensis subsp. Kurstaki strain EG7841+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA-11+SX, Bacillus thuringiensis subsp. Kurstaki strain SA-12+SX, Bacillus thuringiensis subsp. Tenebriosis strain NB176+SX, Bacillus thuringiensis subsp. Thuringiensis strain MPPL002+SX, Bacillus thuringiensis subsp. morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis serotype strain H-14+SX, Bacillus thuringiensis var. japonensis strain Buibui+SX, Bacillus thuringiensis var. san diego strain M-7+SX, Bacillus thuringiensis var. 7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PPAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pnl+SX, Pasteuria penetrans+SX, Pasteuria usgae+SX, Pasteuria thornei+SX, Serratia entomophila+SX, Verticillium chlamydosporium+SX, Verticillium lecani strain NCIM1312+SX, Wolbachia pipientis+SX.

Combination of the Present ingredient in the above Group (b) and the Compound of the present invention:
acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthiavalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid+SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroinconazide+SX, chloroneb+SX, chlorothalonil+SX, chlozolinate+SX, colletochlorin B+SX, copper(II) acetate+SX, copper(II) hydroxide+SX, copper oxychloride+SX, copper(II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, dimethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper(II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract of Allium sativum+SX, extract of the cotyledons of lupine plantlets (“BLAD”)+SX, extract of Equisetum arvense+SX, extract of Melaleuca alternifolia+SX, extract of Reynoutria sachalinensis+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, flubeneteram+SX, fludioxonil+SX, flufenoxadiazam+SX, flufenoxystrobin+SX, fluindapyr+SX, flumetylsulforim+SX, flumorph+SX, fluopicolide+SX, fluopyram+SX, fluopimomide+SX, fluoroimide+SX, fluoxapiprolin+SX, fluoxastrobin+SX, fluoxytioconazole+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metarylpicoxamid+SX, metconazole+SX, methasulfocarb+SX, metiram+SX, metominostrobin+SX, metrafenone+SX, metyltetraprole+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconazole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, seboctylamine+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, N′-{4-[(4,5-dichlorothiazol-2-yl)oxy]-2,5-dimethylphenyl}-N-ethyl-N-methylmethanimidamide (929908-57-6)+SX, N′-(2,5-dimethyl-4-phenoxyphenyl)-N-ethyl-N-methylmethanimidamide (1052688-31-9)+SX, N′-[5-chloro-4-(2-fluorophenoxy)-2-methylphenyl]-N-ethyl-N-methylmethanimidamide (2055589-28-9)+SX, N′-[2-chloro-4-(2-fluorophenoxy)-5-methylphenyl]-N-ethyl-N-methylmethanimidamide (2055756-21-1)+SX, N′-(2-chloro-4-phenoxy-5-methylphenyl)-N-ethyl-N-methylmethanimidamide (2062599-39-5)+SX, N′-[4-(1-hydroxy-1-phenyl-2,2,2-trifluoroethyl)-2-methyl-5-methoxyphenyl]-N-isopropyl-N-methylmethanimidamide (2101814-55-3)+SX, N′-[5-bromo-6-(1-methyl-2-propoxyethoxy)-2-methylpyridin-3-yl]-N-ethyl-N-methylmethanimidamide (1817828-69-5)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R,2S,5S)-5-(4-chlorobenzyl)-2-(chloromethyl)-2-methyl-1-(1H-1,2,4-triazol-1-ylmethyl)cyclopentan-1-ol (1801930-06-2)+SX, (1S,2R, 5R)-5-(4-chlorobenzyl)-2-(chloromethyl)-2-methyl-1-(1H-1,2,4-triazol-1-ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-1-ol (1394057-13-6)+SX, (1R,2S,5S)-2-(chloromethyl)-5-(4-fluorobenzyl)-2-methyl-1-(1H-1,2,4-triazol-1-ylmethyl)cyclopentan-1-ol (1801930-08-4)+SX, (1S,2R,5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-1-carboxylate (1791398-02-1)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)+SX, 1-[2-(1-chlorocyclopropyl)-3-(2,3-difluorophenyl)-2-hydroxypropyl]-1H-imidazole-5-carbonitrile (2018317-25-2)+SX, 2-[6-(4-bromophenoxy)-2-(trifluoromethyl)pyridin-3-yl]-1-(1H-1,2,4-triazol-1-yl)propan-2-ol (2082661-43-4)+SX, 2-[6-(4-chlorophenoxy)-2-(trifluoromethyl)pyridin-3-yl]-1-(1H-1,2,4-triazol-1-yl)propan-2-ol (2082660-27-1)+SX, methyl ({2-methyl-5-[l-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460-05-2)+SX, (2E,3Z)-5-{[l-(4-chlorophenyl)-1H-pyrazol-3-yl]oxy}-2-(methoxyimino)-N,3-dimethylpent-3-enamide (1445331-27-0)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9)+SX, N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxazolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX, N-ethyl-2-methyl-N-({4-[5-(trifluoromethyl)-1,2,4-oxadiazol-3-yl]phenyl}methyl)propanamide+SX, N,2-dimethoxy-N-({4-[5-(trifluoromethyl)-1,2,4-oxadiazol-3-yl]phenyl}methyl)propanamide+SX, N-methoxy-N-({4-[5-(trifluoromethyl)-1,2,4-oxadiazol-3-yl]phenyl}methyl)cyclopropanecarboxamide+SX, N-methoxy-N′-methyl-N-({4-[5-(trifluoromethyl)-1,2,4-oxadiazol-3-yl]phenyl}methyl)urea+SX, N′-ethyl-N-methoxy-N-({4-[5-(trifluoromethyl)-1,2,4-oxadiazol-3-yl]phenyl}methyl)urea+SX, N,N′-dimethoxy-N-({4-[5-(trifluoromethyl)-1,2,4-oxadiazol-3-yl]phenyl}methyl)urea+SX, N-acetyl-2-(ethanesulfonyl)-N-[2-(methoxycarbonyl)-4-(trifluoromethoxy)phenyl]-4-(trifluoromethyl)benzamide (2043675-28-9)+SX, 3-(4-bromo-7-fluoroindol-1-yl)butan-2-yl N-[(3-hydroxy-4-methoxypyridin-2-yl)carbonyl]-L-alaninate+SX, 3-(7-bromoindol-1-yl)butan-2-yl N-[(3-hydroxy-4-methoxypyridin-2-yl)carbonyl]-L-alaninate+SX, 3-(7-bromo-4-fluoroindol-1-yl)butan-2-yl N-[(3-hydroxy-4-methoxypyridin-2-yl)carbonyl]-L-alaninate+SX, 3-(3,5-dichloropyridin-2-yl)butan-2-yl N-[(3-hydroxy-4-methoxypyridin-2-yl)carbonyl]-L-alaninate+SX, 3-(3,5-dichloropyridin-2-yl)butan-2-yl N-{[3-(acetoxymethoxy)-4-methoxypyridin-2-yl]carbonyl}-L-alaninate+SX, (1S)-1-[1-(naphthalen-1-yl)cyclopropyl]ethyl N-[(3-hydroxy-4-methoxypyridin-2-yl)carbonyl]-L-alaninate+SX, (1S)-1-[1-(naphthalen-1-yl)cyclopropyl]ethyl N-[(3-acetoxy-4-methoxypyridin-2-yl)carbonyl]-L-alaninate+SX, (1S)-1-[1-(naphthalen-1-yl)cyclopropyl]ethyl N-{[3-(acetoxymethoxy)-4-methoxypyridin-2-yl]carbonyl}-L-alaninate+SX, Agrobacterium radiobactor strain K1026+SX, N-({4-[5-(trifluoromethyl)-1,2,4-oxadiazol-3-yl]phenyl}methyl)cyclopropanecarboxamide+SX, N-allyl-N-({4-[5-(trifluoromethyl)-1,2,4-oxadiazol-3-yl]phenyl}methyl)acetamide+SX, N-allyl-N-({4-[5-(trifluoromethyl)-1,2,4-oxadiazol-3-yl]phenyl}methyl)propanamide+SX, N-({4-[5-(trifluoromethyl)-1,2,4-oxadiazol-3-yl]phenyl}methyl)propanamide+SX, 3,3,3-trifluoro-N-({2-fluoro-4-[5-(trifluoromethyl)-1,2,4-oxadiazol-3-yl]phenyl}methyl)propanamide+SX, 3,3,3-trifluoro-N-({3-fluoro-4-[5-(trifluoromethyl)-1,2,4-oxadiazol-3-yl]phenyl}methyl)propanamide+SX, 3,3,3-trifluoro-N-({2,3-difluoro-4-[5-(trifluoromethyl)-1,2,4-oxadiazol-3-yl]phenyl}methyl)propanamide+SX, N-({2,3-difluoro-4-[5-(trifluoromethyl)-1,2,4-oxadiazol-3-yl]phenyl}methyl)butanamide+SX, N-methoxy-N-methyl-N′-({4-[5-(trifluoromethyl)-1,2,4-oxadiazol-3-yl]phenyl}methyl)urea+SX, N,N-diethyl-N′-({4-[5-(trifluoromethyl)-1,2,4-oxadiazol-3-yl]phenyl}methyl)urea+SX, N-methyl-N′-({4-[5-(trifluoromethyl)-1,2,4-oxadiazol-3-yl]phenyl}methyl)urea+SX, 1-({4-[5-(trifluoromethyl)-1,2,4-oxadiazol-3-yl]phenyl}methyl)pyrrolidin-2-one+SX, 1-({4-[5-(trifluoromethyl)-1,2,4-oxadiazol-3-yl]phenyl}methyl)piperidin-2-one+SX, 4-({4-[5-(trifluoromethyl)-1,2,4-oxadiazol-3-yl]phenyl}methyl)morpholin-3-one+SX, 2-({4-[5-(trifluoromethyl)-1,2,4-oxadiazol-3-yl]phenyl}methyl)isoxazolidin-3-one+SX, 3,3-dimethyl-1-({4-[5-(trifluoromethyl)-1,2,4-oxadiazol-3-yl]phenyl}methyl)piperidin-2-one+SX, 2-({4-[5-(trifluoromethyl)-1,2,4-oxadiazol-3-yl]phenyl}methyl)-1,2-oxazinan-3-one+SX, 1-({3-fluoro-4-[5-(trifluoromethyl)-1,2,4-oxadiazol-3-yl]phenyl}methyl)azepan-2-one+SX, 4,4-dimethyl-1-({4-[5-(trifluoromethyl)-1,2,4-oxadiazol-3-yl]phenyl}methyl)pyrrolidin-2-one+SX, 5-methyl-1-({4-[5-(trifluoromethyl)-1,2,4-oxadiazol-3-yl]phenyl}methyl)pyrrolidin-2-one+SX, ethyl 1-({4-[5-(trifluoromethyl)-1,2,4-oxadiazol-3-yl]phenyl}methyl)-1H-pyrazole-4-carboxylate+SX, N-methyl-1-({4-[5-(trifluoromethyl)-1,2,4-oxadiazol-3-yl]phenyl}methyl)-1H-pyrazole-4-carboxamide+SX, N-propyl-1-({4-[5-(trifluoromethyl)-1,2,4-oxadiazol-3-yl]phenyl}methyl)-1H-pyrazole-4-carboxamide+SX, N-methoxy-1-({4-[5-(trifluoromethyl)-1,2,4-oxadiazol-3-yl]phenyl}methyl)-1H-pyrazole-4-carboxamide+SX, N-methoxy-N-methyl-1-({4-[5-(trifluoromethyl)-1,2,4-oxadiazol-3-yl]phenyl}methyl)-1H-pyrazole-4-carboxamide+SX, N,N-dimethyl-1-({4-[5-(trifluoromethyl)-1,2,4-oxadiazol-3-yl]phenyl}methyl)-1H-1,2,4-triazol-3-amine+SX, N-methyl-4-[5-(trifluoromethyl)-1,2,4-oxadiazol-3-yl]benzamide+SX, methyl 2-[2-chloro-4-(4-chlorophenoxy)phenyl]-2-hydroxy-3-(1H-1,2,4-triazol-1-yl)propanoate+SX, ethyl 2-[2-chloro-4-(4-chlorophenoxy)phenyl]-2-hydroxy-3-(1H-1,2,4-triazol-1-yl)propanoate+SX, methyl 2-[2-(trifluoromethyl)-4-(4-chlorophenoxy)phenyl]-2-hydroxy-3-(1H-1,2,4-triazol-1-yl)propanoate+SX, 1-(2,3-dimethylpyridin-5-yl)-4,4-difluoro-3,3-dimethyl-3,4-dihydroisoquinoline+SX, 1-[2-(difluoromethyl)-3-methylpyridin-5-yl]-4,4-difluoro-3,3-dimethyl-3,4-dihydroisoquinoline+SX, 2,2-difluoro-N-[6-({[1-(1-methyl-1H-tetrazol-5-yl)benzimidazo]-2-yl]oxy}methyl)pyridin-2-yl]-2-phenoxyacetamide+SX, 1-[2-(1-chlorocyclopropyl)-3-(3-chloro-2-fluorophenyl)-2-hydroxypropyl]-1H-imidazole-5-carbonitrile+SX, ethyl 1-[(4-{[2-(trifluoromethyl)-1,3-dioxolan-2-yl]methoxy}phenyl)methyl]-1H-pyrazole-4-carboxylate+SX, ethyl 1-[(4-{[(1Z)-2-ethoxy-3,3,3-trifluoro-1-propen-1-yl]oxy}phenyl)methyl]-1H-pyrazole-4-carboxylate+SX, 6-chloro-3-(3-cyclopropyl-2-fluorophenoxy)-N-[2-(2,4-dimethylphenyl)-2,2-difluoroethyl]-5-methylpyridazine-4-carboxamide+SX, 6-chloro-3-(3-cyclopropyl-2-fluorophenoxy)-N-[2-(3,4-dimethylphenyl)-2,2-difluoroethyl]-5-methylpyridazine-4-carboxamide+SX, 6-chloro-N-[2-(2-chloro-4-methylphenyl)-2,2-difluoroethyl]-3-(3-cyclopropyl-2-fluorophenoxy)-5-methylpyridazine-4-carboxamide+SX, 2-[cyano(2,6-difluoropyridin-4-yl)amino]-N-(2,2-dimethylcyclobutyl)-5-methylthiazole-4-carboxamide+SX, 2-[cyano(2,6-difluoropyridin-4-yl)amino]-N-(spiro[3.4]octan-1-yl)-5-methylthiazole-4-carboxamide+SX, 2-[cyano(2,6-difluoropyridin-4-yl)amino]-N-hexyl-5-methylthiazole-4-carboxamide+SX, 2-[acetyl(2,6-difluoropyridin-4-yl)amino]-N-(2,2-dimethylcyclobutyl)-5-methylthiazole-4-carboxamide+SX, 2-[(2-methoxyacetyl) (2,6-difluoropyridin-4-yl)amino]-N-(2,2-dimethylcyclobutyl)-5-methylthiazole-4-carboxamide+SX, 2-[(2-methylpropanoyl) (2,6-difluoropyridin-4-yl)amino]-N-(2,2-dimethylcyclobutyl)-5-methylthiazole-4-carboxamide+SX, 2-[(2,6-difluoropyridin-4-yl)amino]-N-(2,2-dimethylcyclobutyl)-5-methylthiazole-4-carboxamide+SX, 2-{[(oxetan-3-yl)carbonyl](2,6-difluoropyridin-4-yl)amino}-N-(2,2-dimethylcyclobutyl)-5-methylthiazole-4-carboxamide+SX, 2-{[(oxolan-3-yl)carbonyl](2,6-difluoropyridin-4-yl)amino}-N-(2,2-dimethylcyclobutyl)-5-methylthiazole-4-carboxamide+SX, 2-{[(oxan-4-yl)carbonyl](2,6-difluoropyridin-4-yl)amino}-N-(2,2-dimethylcyclobutyl)-5-methylthiazole-4-carboxamide+SX, 2-[(2,6-difluoropyridin-4-yl)amino]-N-(2,2-dimethylcyclobutyl)-5-methylthiazole-4-carboxamide+SX, 2-{[(oxetan-3-yl)carbonyl](2,6-difluoropyridin-4-yl)amino}-N-(2,2-dimethylcyclobutyl)-5-methylthiazole-4-carboxamide+SX, 2-{[(oxolan-3-yl)carbonyl](2,6-difluoropyridin-4-yl)amino}-N-(2,2-dimethylcyclobutyl)-5-methylthiazole-4-carboxamide+SX, 2-{[(oxan-4-yl)carbonyl](2,6-difluoropyridin-4-yl)amino}-N-(2,2-dimethylcyclobutyl)-5-methylthiazole-4-carboxamide+SX, 5-[5-(difluoromethyl)-1,3,4-oxadiazol-2-yl]-N-[1-(2-fluorophenyl)ethyl]pyrimidin-2-amine+SX, 5-[5-(difluoromethyl)-1,3,4-oxadiazol-2-yl]-N-[1-(2,6-difluorophenyl)ethyl]pyrimidin-2-amine+SX, 5-[5-(difluoromethyl)-1,3,4-oxadiazol-2-yl]-N-[1-(3,5-difluorophenyl)ethyl]pyrimidin-2-amine+SX, 5-[5-(difluoromethyl)-1,3,4-oxadiazol-2-yl]-N-[1-(6-chloropyridin-3-yl)ethyl]pyrimidin-2-amine+SX, 5-[5-(difluoromethyl)-1,3,4-oxadiazol-2-yl]-N-[1-(2-fluorophenyl)cyclopropyl]pyrimidin-2-amine+SX, 5-[5-(difluoromethyl)-1,3,4-oxadiazol-2-yl]-N-[1-(2,6-difluorophenyl)cyclopropyl]pyrimidin-2-amine+SX, 5-[5-(difluoromethyl)-1,3,4-oxadiazol-2-yl]-N-[1-(2-fluoro-3-methoxyphenyl)cyclopropyl]pyrimidin-2-amine+SX, 5-[5-(difluoromethyl)-1,3,4-oxadiazol-2-yl]-2-{[1-(2,6-difluorophenyl)cyclopropyl]oxy}pyrimidine+SX, 3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, (5S)-3-[3-(3-cyclopropyl-2-fluorophenoxy)-6-methylpyridazin-4-yl]-5-[(2,4-dimethylphenyl)methyl]-5,6-dihydro-4H-1,2,4-oxadiazine+SX, 3-[3-(3-chloro-2-fluorophenoxy)-6-methylpyridazin-4-yl]-5-[(4-bromo-2-methylphenyl)methyl]-5,6-dihydro-4H-1,2,4-oxadiazine+SX, 3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5S)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, (5R)-3-[3-(3-chloro-2-fluorophenoxy)-6-methylpyridazin-4-yl]-5-[(2-chloro-4-methylphenyl)methyl]-5,6-dihydro-4H-1,2,4-oxadiazine+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}-3-methylbutan-2-yl)-2-methylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}-3-methylbutan-2-yl)-2,2-dimethylpropanamide+SX, N-((2S)-1-{3-[2-(5-fluoro-2-methoxyphenyl)-2-hydroxyethyl]-5-[(E)-1-(isopropoxyimino)ethyl]-2,6-dioxo-3,6-dihydropyrimidin-1(2H)-yl}propan-2-yl)-2-methylpropanamide+SX, N-((2S)-1-{3-[2-(2-methoxyphenyl)-2-hydroxyethyl]-5-[(E)-1-(isopropoxyimino)ethyl]-2,6-dioxo-3,6-dihydropyrimidin-1(2H)-yl}propan-2-yl)-2-methylpropanamide+SX, N-((2S)-1-{3-[2-(5-fluoro-2-methoxyphenyl)-2-(2-cyanoethoxy)ethyl]-5-[1-(isopropoxyimino)ethyl]-2,6-dioxo-3,6-dihydropyrimidin-1(2H)-yl}propan-2-yl)-2-methylpropanamide+SX, methyl({5-[1-(2,6-difluoro-4-isopropylphenyl)-1H-pyrazol-3-yl]-2-methylphenyl}methyl)carbamate+SX, methyl({5-[1-(2,6-difluoro-4-cyclopropylphenyl)-1H-pyrazol-3-yl]-2-methylphenyl}methyl)carbamate+SX, methyl({5-[1-(2,6-difluoro-4-methoxyphenyl)-1H-pyrazol-3-yl]-2-methylphenyl}methyl)carbamate+SX, methyl (Z)-2-(5-cyclopentyl-2-methylphenoxy)-3-methoxyprop-2-enoate+SX, methyl (Z)-2-(5-cyclohexyl-2-methylphenoxy)-3-methoxyprop-2-enoate+SX, methyl (Z)-2-[(3-isopropyl-1H-pyrazol-1-yl)-2-methylphenoxy]-3-methoxyprop-2-enoate+SX, 1-(4,5-dimethyl-1H-benzimidazol-1-yl)-4,4-difluoro-3,3-dimethyl-3,4-dihydroisoquinoline+SX, 1-(4,5-dimethyl-1H-benzimidazol-1-yl)-4,4,5-trifluoro-3,3-dimethyl-3,4-dihydroisoquinoline+SX, 1-(pyrazolo[1,5-a]pyridin-3-yl)-4,4-difluoro-3,3-dimethyl-3,4-dihydroisoquinoline+SX, 1-(6,7-dimethylpyrazolo[1,5-a]pyridin-3-yl)-6-fluoro-3,3-dimethylisoquinolin-4(3H)-one+SX, methyl (2Z)-3-methoxy-2-[(4-methyl[1,1′-biphenyl]-3-yl)oxy]prop-2-enoate+SX, Agrobacterium radiobactor strain K84+SX, Bacillus amyloliquefaciens strain PTA-4838 (Aveo (registered trademark) EZ Nematicide)+SX, Bacillus amyloliquefaciens strain AT332+SX, Bacillus amyloliquefaciens strain B3+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MBI600+SX, Bacillus amyloliquefaciens strain QST713+SX, Bacillus amyloliquefaciens isolate strain B246+SX, Bacillus amyloliquefaciens strain F727+SX, Bacillus amyloliquefaciens subsp. plantarum strain D747+SX, Bacillus licheniformis strain HB-2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BS03+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain 0+SX, Candida saitoana+SX, Chaetomium cupreum+SX, Clonostachys rosea+SX, Coniothyrium minitans strain CGMCC8325+SX, Coniothyrium minitans strain CON/M/91-8+SX, cryptococcus albidus+SX, Erwinia carotovora subsp. carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A506+SX, Pseudomonas fluorescens strain CL145A+SX, Pseudomonas fluorescens strain G7090+SX, Pseudomonas sp. strain CAB-02+SX, Pseudomonas syringae strain 742RS+SX, Pseudomonas syringae strain MA-4+SX, Pseudozyma flocculosa strain PF-A22UL+SX, Pseudomonas rhodesiae strain HAI-0804+SX, Pythium oligandrum strain DV74+SX, Pythium oligandrum strain Ml+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICC012+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SCl+SX, Trichoderma atroviride strain SKT-1+SX, Trichoderma atroviride strain T1l+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR-Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, trichoderma stromaticum+SX, Trichoderma virens strain G-41+SX, Trichoderma virens strain GL-21+SX, Trichoderma viride+SX, Variovorax paradoxus strain CGF4526+SX, Harpin protein+SX.

Combination of the Present ingredient in the above Group (c) and the Compound of the present invention:
1-methylcyclopropene+SX, 1,3-diphenylurea+SX, 2,3,5-triiodobenzoic acid+SX, IAA ((1H-indol-3-yl)acetic acid)+SX, IBA (4-(1H-indol-3-yl)butyric acid)+SX, MCPA (2-(4-chloro-2-methylphenoxy) acetic acid)+SX, MCPB (4-(4-chloro-2-methylphenoxy)butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, anisiflupurin+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, paclobutrazol+SX, pendimethalin+SX, prohexadione-calcium+SX, prohydrojasmon+SX, pyraflufen-ethyl+SX, sintofen+SX, sodium 1-naphthaleneacetate+SX, sodium cyanate+SX, thidiazuron+SX, triapenthenol+SX, tribufos+SX, trinexapac-ethyl+SX, uniconazole-P+SX, 2-(naphthalen-1-yl)acetamide+SX, [4-oxo-4-(2-phenylethyl)amino]butylate+SX, methyl 5-(trifluoromethyl)benzo[b]thiophene-2-carboxylate+SX, 3-[(6-chloro-4-phenylquinazolin-2-yl)amino]propan-1-ol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TA-11+SX, Bradyrhizobium japonicum USDA 110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, Zucchini Yellow Mosaik Virus weak strain+SX.

Combination of the Present ingredient in the above Group (d) and the Compound of the present invention:
anthraquinone+SX, deet+SX, icaridin+SX.

The ratio of the Compound of the present invention to the Present ingredient includes, but not limited thereto, as a ratio by weight (the Compound of the present invention:the Present ingredient) 1,000:1 to 1:1,000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, and 1:50, and the others.

The Compound of the present invention has control effect on harmful arthropods such as harmful insects and harmful mites, harmful nematodes, and harmful mollusks. Examples of the harmful arthropods, harmful nematodes, and harmful mollusks include the followings.

### Hemiptera:

from the family Delphacidae, for example, small brown planthopper (Laodelphax striatellus), brown planthopper (Nilaparvata lugens), white-backed planthopper (Sogatella furcifera), corn planthopper (Peregrinus maidis), cereal leafhopper (Javesella pellucida), sugarcane leafhopper (Perkinsiella saccharicida), white-bellied planthopper (Stenocranus pacificus) and Tagosodes orizicolus; from the family Cicadellidae, for example, green rice leafhopper (Nephotettix cincticeps), green paddy leafhopper (Nephotettix virescens), rice leafhopper (Nephotettix nigropictus), zigzag-striped leafhopper (Recilia dorsalis), tea green leafhopper (Empoasca onukii), potato leafhopper (Empoasca fabae), corn leafhopper (Dalbulus maidis), rice leafhopper (Cofana spectra), and Amrasca biguttula biguttula;
from the family Aphrophoridae, for example, European spittlebug (Philaenus spumarius);
from the family Cercopidae, for example, Mahanarva posticata and Mahanarva fimbriolata;
from the family Aphididae, for example, bean aphid (Aphis fabae), soybean aphid (Aphis glycines), cotton aphid (Aphis gossypii), green apple aphid (Aphis pomi), apple aphid (Aphis spiraecola), green peach aphid (Myzus persicae), leaf-curling plum aphid (Brachycaudus helichrysi), cabbage aphid (Brevicoryne brassicae), rosy apple aphid (Dysaphis plantaginea), false cabbage aphid (Lipaphis erysimi), potato aphid (Macrosiphum euphorbiae), foxglove aphid (Aulacorthum solani), lettuce aphid (Nasonovia ribisnigri), grain aphid (Rhopalosiphum padi), corn aphid (Rhopalosiphum maidis), brown citrus aphid (Toxoptera citricida), mealy plum aphid (Hyalopterus pruni), cane aphid (Melanaphis sacchari), black rice root aphid (Tetraneura nigriabdominalis), sugarcane cottony aphid (Ceratovacuna lanigera), apple woolly aphid (Eriosoma lanigerum), and English grain aphid (Sitobion avenae);
from the family Phylloxeridae, for example, grapevine phylloxera (Daktulosphaira vitifoliae), Pecan phylloxera (Phylloxera devastatrix), Pecan leaf Phylloxera (Phylloxera notabilis), and Southern pecan leaf phylloxera (Phylloxera russelae);
from the family Adelgidae, for example, hemlock woolly aphid (Adelges tsugae), balsam woolly aphid (Adelges piceae), and Aphrastasia pectinatae;
from the family Pentatomidae, for example, black rice bug (Scotinophara lurida), black paddy bug (Scotinophara coarctata), common green stink bug (Nezara antennata), white-spotted spined bug (Eysarcoris aeneus), lewis spined bug (Eysarcoris lewisi), white-spotted bug (Eysarcoris ventralis), Eysarcoris annamita, brown marmorated stink bug (Halyomorpha halys), green plant bug (Nezara viridula), brown stink bug (Euschistus heros), red banded stink bug (Piezodorus guildinii), legume stink bug (Piezodorus hybneri), Oebalus pugnax, and Dichelops melacanthus; from the family Cydnidae, for example, Scaptocoris castanea; from the family Alydidae, for example, bean bug (Riptortus clavatus), corbett rice bug (Leptocorisa chinensis), and rice bug (Leptocorisa acuta);
from the family Coreidae, for example, Cletus punctiger and Australian leaf-footed bug (Leptoglossus australis); from the family Lygaeidae, for example, oriental chinch bug (Cavelerius saccharivorus), seed bug (Togo hemipterus), and chinch bug (Blissus leucopterus);
from the family Miridae, for example, rice leaf bug (Trigonotylus caelestialium), sorghum plant bug (Stenotus rubrovittatus), wheat leaf bug (Stenodema calcarata), and American tarnished plant bug (Lygus lineolaris);
from the family Aleyrodidae, for example, greenhouse whitefly (Trialeurodes vaporariorum), tobacco whitefly (Bemisia tabaci), citrus whitefly (Dialeurodes citri), citrus spiny whitefly (Aleurocanthus spiniferus), tea spiny whitefly (Aleurocanthus camelliae), and Pealius euryae; from the family Diaspididae, for example, Abgrallaspis cyanophylli, red scale (Aonidiella aurantii), San Jose scale (Diaspidiotus perniciosus), white peach scale (Pseudaulacaspis pentagona), arrowhead scale (Unaspis yanonensis), and citrus snow scale (Unaspis citri); from the family Coccidae, for example, pink wax scale (Ceroplastes rubens);
from the family Margarodidae, for example, fluted scale (Icerya purchasi) and seychelles fluted scale (Icerya seychellarum);
from the family Pseudococcidae, for example, solanum mealybug (Phenacoccus solani), cotton mealybug (Phenacoccus solenopsis), Japanese mealybug (Planococcus kraunhiae), white peach scale (Pseudococcus comstocki), citrus mealybug (Planococcus citri), currant mealybug (Pseudococcus calceolariae), long-tailed mealybug (Pseudococcus longispinus), and tuttle mealybug (Brevennia rehi); from the family Psyllidae, for example, citrus psylla (Diaphorina citri), two-spotted citrus psyllid (Trioza erytreae), pear sucker (Cacopsylla pyrisuga), Cacopsylla chinensis, potato psyllid (Bactericera cockerelli), and Pear psylla (Cacopsylla pyricola);
from the family Tingidae, for example, sycamore lace bug (Corythucha ciliata), aster tingid (Corythucha marmorata), Japanese pear lace bug (Stephanitis nashi), and azalea lace bug (Stephanitis pyrioides);
from the family Cimicidae, for example, common bed bug (Cimex lectularius) and tropical bed bug (Cimex hemipterus); from the family Cicadidae, for example, Quesada gigas; from the family Reduviidae, for example, Triatoma infestans, large kissing bug (Triatoma rubrofasciata), Triatoma dimidiata, and Rhodonius prolixus;
and the others.

### Lepidoptera:

from the family Crambidae, for example, rice stem borer (Chilo suppressalis), Dark-headed stem borer (Chilo polychrysus), white stem borer (Scirpophaga innotata), yellow paddy borer (Scirpophaga incertulas), Rupela albina, rice leaf roller (Cnaphalocrocis medinalis), Marasmia patnalis, rice leaf roller (Marasmia exigua), cotton leaf roller (Notarcha derogata), corn borer (Ostrinia furnacalis), European corn borer (Ostrinia nubilalis), cabbage webworm (Hellula undalis), grape leafroller (Herpetogramma luctuosale), bluegrass webworm (Parapediasia teterrellus), rice case-worm (Nymphula depunctalis), Sugarcane borer (Diatraea saccharalis), and eggplant fruit borer (Leucinodes orbonalis);
from the family Pyralidae, for example, lesser cornstalk borer (Elasmopalpus lignosellus), mealworm moth (Plodia interpunctella), persimmon bark borer (Euzophera batangensis), and fig moth (Cadra cautella);
from the family Noctuidae, for example, common cutworm (Spodoptera litura), beet armyworm (Spodoptera exigua), rice armyworm (Mythimna separata), cabbage moth (Mamestra brassicae), pink borer (Sesamia inferens), grass armyworm (Spodoptera mauritia), green rice caterpillar (Naranga aenescens), Spodoptera frugiperda, true armyworm (Spodoptera exempta), semitropical armyworm (Spodoptera eridania), black cutworm (Agrotis ipsilon), turnip moth (Agrotis segetum), beet worm (Autographa nigrisigna), rice looper (Plusia festucae), Soybean looper (Chrysodeixis includens), Trichoplusia spp., Heliothis spp. (such as tobacco budworm (Heliothis virescens)), Helicoverpa spp. (such as tobacco budworm (Helicoverpa armigera), corn earworm (Helicoverpa zea)), velvet-bean caterpillar (Anticarsia gemmatalis), cotton leafworm (Alabama argillacea), and hop vine borer (Hydraecia immanis);
from the family Pieridae, for example, common cabbage worm (Pieris rapae);
from the family Tortricidae, for example, oriental fruit moth (Grapholita molesta), Grapholita dimorpha, soybean moth (Leguminivora glycinivorella), Matsumuraeses azukivora, summer fruit tortrix (Adoxophyes orana fasciata), smaller tea tortrix (Adoxophyes honmai), Japanese tea tortrix (Homona magnanima), apple tortrix (Archips fuscocupreanus), codling moth (Cydia pomonella), sugarcane shoot borer (Tetramoera schistaceana), bean shoot borer (Epinotia aporema), citrus fruit borer (Citripestis sagittiferella), and European grapevine moth (Lobesia botrana);
from the family Gracillariidae, for example, tea leaf roller (Caloptilia theivora) and Asiatic apple leaf miner (Phyllonorycter ringoniella);
from the family Carposinidae, for example, peach fruit moth (Carposina sasakii);
from the family Lyonetiidae, for example, coffee leaf miner (Leucoptera coffeella), peach leaf miner (Lyonetia clerkella), and Lyonetia prunifoliella;
from the family Lymantriidae, for example, Lymantria spp. (such as gypsy moth (Lymantria dispar)) and Euproctis spp. (such as tea lymantriid (Euproctis pseudoconspersa)); from the family Plutellidae, for example, diamondback moth (Plutella xylostella);
from the family Gelechiidae, for example, peach worm (Anarsia lineatella), sweetpotato leaf folder (Helcystogramma triannulella), pink bollworm (Pectinophora gossypiella), potato moth (Phthorimaea operculella), and tomato leafminer (Tuta absoluta);
from the family Arctiidae, for example, American white moth (Hyphantria cunea);
from the family Castniidae, for example, giant sugarcane borer (Telchin licus);
from the family Cossidae, for example, Cossus insularis; from the family Geometridae, for example, Ascotis selenaria; from the family Limacodidae, for example, blue-striped nettle grub (Parasa lepida);
from the family Stathmopodidae, for example, persimmon fruit moth (Stathmopoda masinissa);
from the family Sphingidae, for example, tobacco hornworm (Acherontia lachesis);
from the family Sesiidae, for example, Nokona feralis, cherry borer (Synanthedon hector), and Synanthedon tenuis:
from the family Hesperiidae, for example, rice skipper (Parnara guttata);
from the family Tineidae, for example, casemaking clothes moth (Tinea translucens), common clothes moth (Tineola bisselliella); and the others.

### Thysanoptera:

from the family Thripidae, for example, western flower thrips (Frankliniella occidentalis), oriental thrips (Thrips palmi), yellow tea thrips (Scirtothrips dorsalis), onion thrips (Thrips tabaci), eastern flower thrips (Frankliniella intonsa), rice thrips (Stenchaetothrips biformis), Echinothrips americanus, and avocado thrips (Scirtothrips perseae);
from the family Phlaeothripidae, for example, aculeated rice thrips (Haplothrips aculeatus);
and the others.

### Diptera:

from the family Anthomyiidae, for example, seedcorn maggot (Delia platura), onion maggot (Delia antiqua), and beet leaf miner (Pegomya cunicularia);
from the family Ulidiidae, for example, sugarbeet root maggot (Tetanops myopaeformis);
from the family Agromyzidae, for example, rice leaf miner (Agromyza oryzae), tomato leaf miner (Liriomyza sativae), chrysanthemum leaf miner (Liriomyza trifolii), and pea leafminer (Chromatomyia horticola);
from the family Chloropidae, for example, rice stem maggot (Chlorops oryzae);
from the family Tephritidae, for example, melon fly (Bactrocera cucurbitae), oriental fruit fly (Bactrocera dorsalis), Malaysian fruit fly (Bactrocera latifrons), olive fruit fly (Bactrocera oleae), Queensland fruit fly (Bactrocera tryoni), Mediterranean fruit fly (Ceratitis capitata), apple maggot (Rhagoletis pomonella), and Japanese cherry fruit fly (Rhacochlaena japonica);
from the family Ephydridae, for example, smaller rice leaf miner (Hydrellia griseola), whorl maggot (Hydrellia philippina), and paddy stem maggot (Hydrellia sasakii); from the family Drosophilidae, for example, cherry drosophila (Drosophila suzukii), and common fruit fly (Drosophila melanogaster);
from the family Phoridae, for example, Megaselia spiracularis;
from the family Psychodidae, for example, Clogmia albipunctata;
from the family Sciaridae, for example, Bradysia difformis and Bradysia odoriphaga;
from the family Cecidomyiidae, for example, hessian fly (Mayetiola destructor) and paddy gall fly (Orseolia oryzae); from the family Diopsidae, for example, Diopsis macrophthalma;
from the family Glossinidae, for example, Glossina palpalis and Glossina morsitans;
from the family Simuliidae, for example, Simulium japonicum and Simulium damnosum;
from the subfamily Phlebotominae;
from the family Tipulidae, for example, rice crane fly (Tipula aino), common cranefly (Tipula oleracea), and European cranefly (Tipula paludosa);
from the family Culicidae, for example, common house mosquito (Culex pipiens pallens), Culex tritaeniorhynchus, Culex pipiens f. molestus, brown house mosquito (Culex quinquefasciatus), northern house mosquito (Culex pipiens pipiens), Culex vishnui, Asian tiger mosquito (Aedes albopictus), dengue mosquito (Aedes aegypti), Chinese malaria mosquito (Anopheles sinensis), Anopheles gambiae, Anopheles stephensi, Anopheles coluzzii, Anopheles albimanus, Anopheles sundaicus, Anopheles arabiensis, Anopheles funestus, Anopheles darlingi, Anopheles farauti, and Anopheles minimus;
from the family Simulidae, for example, Prosimulium yezoensis and Simulium ornatum;
from the family Tabanidae, for example, Tabanus trigonus; from the family Muscidae, for example, house fly (Musca domestica), false stable fly (Muscina stabulans), biting house fly (Stomoxys calcitrans), and buffalo fly (Haematobia irritans);
from the family Calliphoridae;
from the family Sarcophagidae;
from the family Chironomidae, for example, Chironomus plumosus, Chironomus yoshimatsui, and Glyptotendipes tokunagai;
from the family Fannidae;
and the others.

### Coleoptera:

from the family Chrysomelidae, for example, Diabrotica spp. (such as western corn rootworm (Diabrotica virgifera virgifera), southern corn rootworm (Diabrotica undecimpunctata howardi), northern corn rootworm (Diabrotica barberi), Mexican corn rootworm (Diabrotica virgifera zeae), banded cucumber beetle (Diabrotica balteata), cucurbit beetle (Diabrotica speciosa)), bean leaf beetle (Cerotoma trifurcata), barley leaf beetle (Oulema melanopus), cucurbit leaf beetle (Aulacophora femoralis), striped flea beetle (Phyllotreta striolata), cabbage flea beetle (Phyllotreta cruciferae), western black flea beetle (Phyllotreta pusilla), cabbage stem flea beetle (Psylliodes chrysocephala), hop flea beetle (Psylliodes punctulata), Colorado potato beetle (Leptinotarsa decemlineata), rice leaf beetle (Oulema oryzae), grape colaspis (Colaspis brunnea), corn flea beetle (Chaetocnema pulicaria), sweet-potato flea beetle (Chaetocnema confinis), potato flea beetle (Epitrix cucumeris), rice leaf beetle (Dicladispa armigera), southern corn leaf beetle (Myochrous denticollis), Laccoptera quadrimaculata, tobacco flea beetle (Epitrix hirtipennis), daikon leaf beetle (Phaedon brassicae) and two-striped leaf beetle (Medythia nigrobilineata);
from the family Carabidae, for example, Seedcorn beetle (Stenolophus lecontei) and slender seed-corn ground beetle (Clivina impressifrons);
from the family Scarabaeidae, for example, cupreus chafer (Anomala cuprea), soybean beetle (Anomala rufocuprea), Anomala albopilosa, Japanese beetle (Popillia japonica), yellowish elongate chafer (Heptophylla picea), European Chafer (Rhizotrogus majalis), Tomarus gibbosus, Holotrichia spp., Phyllophaga spp. (such as June beetle (Phyllophaga crinita)), and Diloboderus spp. (such as Diloboderus abderus);
from the family Anthriibidae, for example, coffee bean weevil (Araecerus coffeae);
from the family Aponidae, for example, sweet-potato weevil (Cylas formicarius);
from the family Bruchidae, for example, Mexican bean weevil (Zabrotes subfasciatus);
from the family Scolytidae, for example, pine beetle (Tomicus piniperda) and coffee berry borer (Hypothenemus hampei);
from the family Curculionidae, for example, West Indian sweet-potato weevil (Euscepes postfasciatus), alfalfa weevil (Hypera postica), maize wevil (Sitophilus zeamais), rice weevil (Sitophilus oryzae), grain weevil (Sitophilus granarius), rice plant weevil (Echinocnemus squameus), rice water weevil (Lissorhoptrus oryzophilus), Rhabdoscelus lineaticollis, boll weevil (Anthonomus grandis), nunting billbug (Sphenophorus venatus), southern corn billbug (Sphenophorus callosus), soybean stalk weevil (Sternechus subsignatus), sugarcane weevil (Sphenophorus levis), rusty gourd-shaped weevil (Scepticus griseus), brown gourd-shaped weevil (Scepticus uniformis), Aracanthus spp. (such as Aracanthus mourei), and cotton root borer (Eutinobothrus brasiliensis);
from the family Tenebrionidae, for example, red meal beetle (Tribolium castaneum), mason beetle (Tribolium confusum), and lesser mealworm (Alphitobius diaperinus);
from the family Coccinellidae, for example, twenty-eight-spotted ladybird (Epilachna vigintioctopunctata); from the family Bostrychidae, for example, common powder-post beetle (Lyctus brunneus), and lesser grain borer (Rhizopertha dominica);
from the family Ptinidae;
from the family Cerambycidae, for example, citrus long-horned beetle (Anoplophora malasiaca), Migdolus fryanus, and peach borer (Aromia bungii);
from the family Elateridae, for example, Melanotus okinawensis, barley wireworm (Agriotes fuscicollis), Melanotus legatus, Anchastus spp., Conoderus spp., Ctenicera spp., Limonius spp., and Aeolus spp.;
from the family Staphylinidae, for example, Paederus fuscipes;
from the family Dermestidae, for example, varied carpet beetle (Anthrenus verbasci), hide beetle (Dermestes maculates), and khapra beetle (Trogoderma granarium);
from the family Anobiidae, for example, tobacco beetle (Lasioderma serricorne), and biscuit beetle (Stegobium paniceum);
from the family Laemophloeidae, for example, flat grain beetle (Cryptolestes ferrugineus);
from the family Silvanidae, for example, saw-toothed grain beetle (Oryzaephilus surinamensis);
from the family Nitidulidae, for example, blossom beetle (Brassicogethes aeneus);
and the others.

### Orthoptera:

from the family Acrididae, for example, oriental migratory locust (Locusta migratoria), Moroccan locust (Dociostaurus maroccanus), Australian plague locust (Chortoicetes terminifera), red locust (Nomadacris septemfasciata), brown locust (Locustana pardalina), tree locust (Anacridium melanorhodon), Italian locust (Calliptamus italicus), differential grasshopper (Melanoplus differentialis), two-striped grasshopper (Melanoplus bivittatus), migratory grasshopper (Melanoplus sanguinipes), red-legged grasshopper (Melanoplus femurrubrum), clear-winged grasshopper (Camnula pellucida), desert locust (Schistocerca gregaria), Yellow-winged locust (Gastrimargus musicus), spur-throated locust (Austracris guttulosa), Japanese grasshopper (Oxya yezoensis), rice grasshopper (Oxya japonica), and Bombay locust (Patanga succincta);
from the family Gryllotalpidae, for example, oriental mole cricket (Gryllotalpa orientalis);
from the family Gryllidae, for example, house cricket (Acheta domestica), and emma field cricket (Teleogryllus emma);
from the family Tettigoniidae, for example, mormon cricket (Anabrus simplex);
and the others.

### Hymenoptera:

from the family Tenthredinidae, for example, beet sawfly (Athalia rosae) and nippon cabbage sawfly (Athalia japonica);
from the family Formicidae, for example, Solenopsis spp. (such as red imported fire ant (Solenopsis invicta), tropical fire ant (Solenopsis geminata)), Atta spp. (such as brown leaf-cutting ant (Atta capiguara), Acromyrmex spp., Paraponera clavata, black house ant (Ochetellus glaber), pharaoh ant (Monomorium pharaonis), Argentine ant (Linepithema humile), Formica japonica, Pristomyrmex punctutus, Pheidole noda, big-headed ant (Pheidole megacephala), Camponotus spp. (such as Camponotus japonicus, Camponotus obscuripes), Pogonomyrmex spp. (such as western harvester ant (Pogonomyrmex occidentalis)), Wasmania spp. (such as Wasmania auropunctata), and long-legged ant (Anoplolepis gracilipes);
from the family Vespidae, for example, Asian giant hornet (Vespa mandarinia, Vespa simillima, Vespa analis, Asian hornet (Vespa velutina), and Polistes jokahamae;
from the family Siricidae, for example, pine wood wasp (Urocerus gigas);
from the family Bethylidae;
and the others.

### Blattodea:

from the family Ectobiidae, for example, German cockroach (Blattella germanica);
from the family Blattidae, for example, smoky-brown cockroach (Periplaneta fuliginosa), American cockroach (Periplaneta americana), Australian cockroach (Periplaneta australasiae), brown cockroach (Periplaneta brunnea), and black cockroach (Blatta orientalis);
from the family Termitidae, for example, Japanese termite (Reticulitermes speratus), Formosan termite (Coptotermes formosanus), western drywood termite (Incisitermes minor), Cryptotermes domesticus, Odontotermes formosanus, Neotermes koshunensis, Glyptotermes satsumensis, Glyptotermes nakajimai, Glyptotermes fuscus, Hodotermopsis sjostedti, Coptotermes guangzhouensis, Reticulitermes amamianus, Reticulitermes miyatakei, Reticulitermes kanmonensis, Nasutitermes takasagoensis, Pericapritermes nitobei, Sinocapritermes mushae, and Cornitermes cumulans; and the others.

### Siphonaptera:

from the family Pulicidae, for example, human flea (Pulex irritans), cat flea (Ctenocephalides felis), dog flea (Ctenocephalides canis), oriental rat flea (Xenopsylla cheopis), and chicken flea (Echidnophaga gallinacea); from the family Pulicidae, for example, chigoe flea (Tunga penetrans);
from the family Ceratophyllidae, for example, European rat flea (Nosopsyllus fasciatus); and the others.

### Psocodae:

from the family Pediculidae, for example, head louse (Pediculus humanus capitis);
from the family Pthiridae, for example, crab louse (Pthirus pubis);
from the family Haematopinidae, for example, short-nosed cattle louse (Haematopinus eurysternus) and pig louse (Haematopinus suis);
from the family Linognathidae, for example, blue cattle louse (Linognathus vituli), sheep face louse (Linognathus ovillus), and capillate louse (Solenopotes capillatus);
from the family Bovicoliidae, for example, cattle biting louse (Bovicola bovis), sheep biting louse (Bovicola ovis), Bovicola breviceps, Damalinia forficula, and Werneckiella spp.;
from the family Trichodectidae, for example, dog biting louse (Trichodectes canis) and cat louse (Felicola subrostratus); from the family Menoponidae, for example, common chicken louse (Menopon gallinae), chicken body louse (Menacanthus stramineus), and Trinoton spp;
from the family Trimenoponidae, for example, Cummingsia spp.;
from the family Trogiidae, for example, death watch (Trogium pulsatorium);
from the family Liposcelidae or Liposcelididae, for example, book louse (Liposcelis corrodens), Liposcelis bostrychophila, Liposcelis pearmani and Liposcelis entomophila;
and the other.

### Thysanura:

from the family Lepismatidae, for example, oriental silverfish (Ctenolepisma villosa) and moth fish (Lepisma saccharina);
and the others.

### Acari:

from the family Tetranychidae, for example, common red spider mite (Tetranychus urticae), kanzawa spider mite (Tetranychus kanzawai), red spider mite (Tetranychus evansi), citrus red mite (Panonychus citri), fruit-tree red spider mite (Panonychus ulmi), and Oligonychus spp.;
from the family Eriophyidae, for example, Japanese citrus rust mite (Aculops pelekassi), Phyllocoptruta citri, tomato mite (Aculops lycopersici), purple mite (Calacarus carinatus), tea rust mite (Acaphylla theavagrans), Eriophyes chibaensis, apple bud mite (Aculus schlechtendali), Aceria diospyri, Aceria tosichella, and Shevtchenkella sp.;
from the family Tarsonemidae, for example, broad mite (Polyphagotarsonemus latus);
from the family Tenuipalpidae, for example, Brevipalpus phoenicis;
from the family Tuckerellidae;
from the family Ixodidae, for example, Haemaphysalis longicornis, Haemaphysalis flava, Haemaphysalis japonica, Haemaphysalis campanulata, American dog tick (Dermacentor variabilis), Dermacentor taiwanensis, Rocky Mountain wood tick (Dermacentor andersoni), netted tick (Dermacentor reticulatus), Ixodes ovatus, Ixodes persulcatus, black-legged tick (Ixodes scapularis), Ixodes pacificus, Ixodes holocyclus, Ixodes ricinus, lone star tick (Amblyomma americanum), gulf coast tick (Amblyomma maculatum), Rhipicephalus microplus, cattle tick (Rhipicephalus annulatus), brown dog tick (Rhipicephalus sanguineus), Rhipicephalus appendiculatus, and Rhipicephalus decoloratus; from the family Argasidae, for example, fowl tick (Argas persicus), Ornithodoros hermsi, and Ornithodoros turicata; from the family Acaridae, for example, cereal mite (Tyrophagus putrescentiae) and grassland mite (Tyrophagus similis);
from the family Pyroglyphidae, for example, American house dust mite (Dermatophagoides farinae) and European house dust mite (Dermatophagoides pteronyssinus);
from the family Cheyletidae, for example, Cheyletus eruditus, Cheyletus malaccensis, Chelacaropsis moorei, and Cheyletiella yasguri;
from the family Psoroptidae, for example, sheep scab mite (Psoroptes ovis), horse psoroptic mange mite (Psoroptes equi), Knemidocoptes mutans, ear mange mite (Otodectes cynotis), and Chorioptes spp.;
from the family Sarcoptidae, for example, Notoedres cati, Notoedres muris, and itch mite (Sarcoptes scabiei); from the family Listrophoridae, for example, Listrophorus gibbus;
from the family Dermanyssidae, for example, bird mite (Dermanyssus gallinae);
from the family Macronyssidae, for example, feather mite (Ornithonyssus sylviarum) and tropical rat mite (Ornithonyssus bacoti);
from the family Varroidae, for example, Varroa jacobsoni; from the family Demodicidae, for example, dog follicle mite (Demodex canis) and cat follicle mite (Demodex cati); from the family Trombiculidae, for example, Leptotrombidium akamushi, Leptotrombidium pallidum, and Leptotrombidium scutellare;
and the others.

### Araneae:

from the family Eutichuridae, for example, Cheiracanthium japonicum;
from the family Theridiidae, for example, red-back spider (Latrodectus hasseltii);
and the others

### Polydesmida:

from the family Paradoxosomatidae, for example, flat-backed millipede (Oxidus gracilis) and Nedyopus tambanus; and the others.

### Isopoda:

from the family Armadillidiidae, common pill bug (Armadillidium vulgare);
and the others.

### Chilopoda:

from the family Scutigeridae, for example, Thereuonema hilgendorfi;
from the family Scolopendridae, giant tropical centipede (Scolopendra subspinipes);
from the family Ethopolyidae, Bothropolys rugosus; and the others.

### Gastropoda:

from the family Limacidae, for example, tree slug (Limax marginatus) and garden tawny slug (Limax flavus); from the family Philomycidae, for example, Meghimatium bilineatum;
from the family Ampullariidae, for example, golden apple snail (Pomacea canaliculata);
from the family Lymnaeidae, for example, Austropeplea ollula;
and the others.

### Nematoda:

from the family Aphelenchoididae, for example, rice white-tip nematode (Aphelenchoides besseyi);
from the family Pratylenchidae, for example, root lesion nematode (Pratylenchus coffeae), Pratylenchus brachyurus, California meadow nematode (Pratylenchus neglectus), and Radopholus similis;
from the family Heteroderidae, for example, javanese root-knot nematode (Meloidogyne javanica), southern root-knot nematode (Meloidogyne incognita), guava root-knot nematodes (Meloidogyne enterolobii), northern root-knot nematode (Meloidogyne hapla), Columbia root-knot nematode (Meloidogyne chitwoodi), soybean cyst nematode (Heterodera glycines), potato cyst nematode (Globodera rostochiensis), and white potato cyst nematode (Globodera pallida);
from the family Hoplolaimidae, for example, Rotylenchulus reniformis;
from the family Anguinidae, for example, strawberry bud nematode (Nothotylenchus acris), and stem nematode (Ditylenchus dipsaci);
from the family Tylenchulidae, for example, citrus nematode (Tylenchulus semipenetrans);
from the family Longidoridae, for example, dagger nematode (Xiphinema index);
from the family Trichodoridae;
from the family Parasitaphelenchidae, for example, pine wilt disease (Bursaphelenchus xylophilus);
and the others.

The harmful arthropods such as harmful insects and harmful mites, harmful mollusks and harmful nematodes may be those having a reduced susceptibility to or a developed resistance to an insecticide, a mitecide, a molluscicide or a nematicide.

The method for controlling harmful arthropods of the present invention is carried out by applying an effective amount of the Compound of the present invention or the Composition A to a harmful arthropod directly and/or a habitat thereof (for example, plant bodies, soil, an interior of a house, animal bodies). Examples of the method for controlling harmful arthropods of the present invention include foliage treatment, soil treatment, root treatment, shower treatment, smoking treatment, water surface treatment and seed treatment.

The Compound of the present invention or the Composition A is usually used by mixing it with inert carrier(s) such as solid carrier(s), liquid carrier(s), and gaseous carrier(s), surfactant(s), and the like, and as needed, adding thereto auxiliary agent(s) for formulation such as binder(s), dispersant(s), and stabilizer(s) to be formulated into an aqueous suspension formulation, an oily suspension formulation, an oil solution, an emulsifiable concentrate, an emulsion formulation, a microemulsion formulation, a microcapsule formulation, a wettable powder, a granular wettable powder, a dust formulation, a granule, a tablet, an aerosol formulation, a resin formulation, or the like. In addition to these formulations, the Compound of the present invention or the Composition A may be used by formulating it into a dosage form described in Manual on development and use of FAO and WHO Specifications for pesticides, FAO Plant Production and Protection Papers-271-276, prepared by the FAO/WHO Joint Meeting on Pesticide Specifications, 2016, ISSN:0259-2517.

These formulations usually comprise 0.0001 to 99% by weight ratio of the Compound of the present invention or the Composition A.

Examples of the solid carrier include fine powders and granules of clays (for example, pyrophyllite clay and kaolin clay), talc, calcium carbonate, diatomaceous earth, zeolite, bentonite, acid white clay, attapulgite, white carbon, ammonium sulfate, vermiculite, perlite, pumice, silica sand, chemical fertilizers (for example, ammonium sulfate, ammonium phosphate, ammonium nitrate, urea, and ammonium chloride), and the others; as well as resins (for example, polyethylene, polypropylene, polyester, polyurethane, polyamide, and polyvinyl chloride).

Examples of the liquid carrier include water, alcohols (for example, ethanol, cyclohexanol, benzyl alcohol, propylene glycol, and polyethylene glycol), ketones (for example, acetone and cyclohexanone), aromatic hydrocarbons (for example, xylene, phenyl xylyl ethane, and methylnaphthalene), aliphatic hydrocarbons (for example, hexane and cyclohexane), esters (for example, ethyl acetate, methyl oleate, and propylene carbonate), nitriles (for example, acetonitrile), ethers (for example, ethylene glycol dimethyl ether), amides (for example, N,N-dimethylformamide and N,N-dimethyloctanamide), sulfoxides (for example, dimethyl sulfoxide), lactams (for example, N-methylpyrrolidone and N-octylpyrrolidone), fatty acids (for example, oleic acid), and vegetable oils (for example, soybean oil).

Examples of the gaseous carrier include fluorocarbon, butane gas, LPG (liquefied petroleum gas), dimethyl ether, nitrogen, and carbon dioxide.

Examples of the surfactant include nonionic surfactants (for example, polyoxyethylene alkyl ethers, polyoxyethylene alkyl aryl ethers, and polyethylene glycol fatty acid esters), and anionic surfactants (for example, alkyl sulfonates, alkyl aryl sulfonates, and alkyl sulfates).

Examples of the other auxiliary agent for formulation include binders, dispersants, colorants, and stabilizers, and the specific examples thereof include polysaccharides (for example, starch, gum arabic, cellulose derivatives, and alginic acid), lignin derivatives, water-soluble synthetic polymers (for example, polyvinyl alcohol, polyvinyl pyrrolidone, and polyacrylic acids), acidic isopropyl phosphate, and dibutylhydroxytoluene.

Moreover, some adjuvants can be employed to improve or help the efficacy of the Compound of the present invention. The specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark).

As used herein, examples of the plant include whole plant, stem and leaf, flower, ear, fruit, tree stem, branch, crown, seed, vegetative reproductive organ, and seedling.

A vegetative reproduction organ means a part of plant such as root, stem, and leaf which has a growth capability even when said part is separated from the plant body and placed into soil. Examples of the vegetative reproduction organ include tuberous root, creeping root, bulb, corm or solid bulb, tuber, rhizome, stolon, rhizophore, cane cuttings, propagule, and vine cutting. Stolon is also referred to as “runner”, and propagule is also referred to as “propagulum” and categorized into broad bud and bulbil. Vine cutting means a shoot (collective term of leaf and stem) of sweet potato, glutinous yam, or the like. Bulb, corm or solid bulb, tuber, rhizome, cane cuttings, rhizophore, and tuberous root are also collectively referred to as “bulb”. For example, cultivation of potato starts with planting a tuber into soil, and the tuber to be used is generally referred to as “seed potato”.

Examples of a method for controlling harmful arthropods by applying an effective amount of the Compound of the present invention or the Composition A to soils include a method of applying an effective amount of the Compound of the present invention or the Composition A to soils before planting plants or after planting plants. Specifically, examples of the application method include planting hole treatment (spraying into planting holes, soil mixing after planting hole treatment), plant foot treatment (plant foot spraying, soil mixing after plant foot treatment, irrigation at plant foot, plant foot treatment at a later seeding raising stage), planting furrow treatment (planting furrow spraying, soil mixing after planting furrow treatment), planting row treatment (planting row spraying, soil mixing after planting row treatment, planting row spraying at a growing stage), planting row treatment at the time of sowing (planting row spraying at the time of sowing, soil mixing after planting row treatment at the time of sowing), broadcast treatment (overall soil surface spraying, soil mixing after broadcast treatment), side-article treatment, treatment of water surface (application to water surface, application to water surface after flooding), other soil spraying treatment (spraying of a granular formulation on leaves at a growing stage, spraying under a canopy or around a tree stem, spraying on the soil surface, mixing with surface soil, spraying into seed holes, spraying on the ground surfaces of furrows, spraying between plants), other irrigation treatment (soil irrigation, irrigation at a seedling raising stage, chemical solution injection treatment, irrigation of a plant part just above the ground, chemical solution drip irrigation, chemigation), seedling raising box treatment (spraying into a seedling raising box, irrigation of a seedling raising box, flooding into a seedling raising box with chemical solution), seedling raising tray treatment (spraying on a seedling raising tray, irrigation of a seedling raising tray, flooding into a seedling raising tray with chemical solution), seedbed treatment (spraying on a seedbed, irrigation of a seedbed, spraying on a lowland rice nursery, immersion of seedlings), seedbed soil incorporation treatment (mixing with seedbed soil, mixing with seedbed soil before sowing, spraying at sowing before covering with soils, spraying at sowing after covering with soils, mixing with covering with soils), and other treatment (mixing with culture soil, plowing under, mixing with surface soil, mixing with soil at the place where raindrops fall from a canopy, treatment at a planting position, spraying of a granule formulation on flower clusters, mixing with a paste fertilizer).

Examples of the application to seeds (or seed treatments) include an application of the Compound of the present invention or the Composition A to seeds or vegetative reproductive organs, and specific examples thereof include spraying treatment in which a suspension of the Compound of the present invention or the Composition A is sprayed onto seed surface or the vegetative reproductive organ surface in the form of mist; smearing treatment in which the Compound of the present invention or the Composition A is coated a surface of seeds or the vegetative reproductive organ; a soaking treatment in which the seeds are soaked into the solution of the Compound of the present invention or the Composition A for a certain time; and a method for coating the seeds or the vegetative reproductive organ with a carrier containing the Compound of the present invention or the Composition A (film coating treatment, pellet coating treatment). Examples of the above-described vegetative reproductive organ include particularly seed potato.

When the Composition A is applied to seeds or vegetative reproductive organs, the Composition A may be also applied to seeds or vegetative reproductive organs as a single formulation, or the Composition A may be applied to seeds or vegetative reproductive organs as multiple different formulations by multiple times. Examples of the method in which the Composition A is applied as multiple different formulations by multiple times include, for example, a method in which the formulations comprising as an active component the Compound of the present invention only are applied, and seeds or vegetative reproductive organs are air dried, followed by applying the formulations comprising the Present ingredient; and a method in which the formulations comprising as an active component the Compound of the present invention and the Present ingredients are applied, and seeds or vegetative reproductive organs are air dried, followed by applying the formulations comprising the Present ingredients other than the already-applied Present ingredients, are included.

As used herein, seeds or vegetative reproductive organs carrying the Compound of the present invention or the Composition A means seeds or vegetative reproductive organs in the state where the Compound of the present invention or the Composition A is adhered to a surface of the seeds or the vegetative reproductive organ. The above-described seeds or vegetative reproductive organs carrying the Compound of the present invention or the Composition A may be adhered by any other materials that are different from the Compound of the present invention or the Composition A before or after being adhered the Compound of the present invention or the Composition A to the seeds or vegetative reproductive organs.

Also, when the Composition A is adhered in a form of layer(s) to a surface of seeds or vegetative reproductive organ, the layer(s) is/are composed of one layer or a multiple layers. Also, when multiple layers are formed, each of the layer may be composed of a layer comprising one or more active ingredients, or a combination of a layer comprising one or more active ingredients and a layer not comprising an active ingredient.

Seeds or vegetative reproductive organs carrying the Compound of the present invention or the Composition A can be obtained, for example, by applying the formulations comprising the Compound of the present invention or the Composition A by the above-described application method to seeds to seeds or vegetative reproductive organs. When the Compound of the present invention or the Composition A is applied for harmful arthropods control in agricultural fields, the application dose thereof is usually within a range of 1 to 10,000 g of the Compound of the present invention per 10,000 m². In the case of being applied to seeds or vegetative reproductive organs, the dose of application dose thereof is usually within a range of 0.001 to 100 g of the Compound of the present invention per 1 Kg of seeds. When the Compound of the present invention or the Composition A is formulated into an emulsifiable concentrate, a wettable powder or a flowable etc., they are usually applied by diluting them with water so as to make an effective concentration of the active ingredients 0.01 to 10,000 ppm, and the dust formulation or the granular formulation, etc., is usually applied as itself without diluting them.

Also, the resin preparation which is processed into a sheet or a string may be applied by winding a plant with a sheet or a string of the resin preparation, putting a string of the resin preparation around a crop so that the plant is surrounded by the string, or laying a sheet of the resin preparation on the soil surface near the root of a plant.

When the Compound of the present invention or the Composition A is used to control harmful arthropods that live inside a house, the application dose as an amount of the Compound of the present invention is usually within a range from 0.01 to 1,000 mg per 1 m² of an area to be treated, in the case of using it on a planar area. In the case of using it spatially, the application dose as an amount of the Compound of the present invention is usually within a range from 0.01 to 500 mg per 1 m³ of the space to be treated. When the Compound of the present invention or the Composition A is formulated into emulsifiable concentrates, wettable powders, flowables or the others, such formulations are usually applied after diluting it with water in such a way that a concentration of the active ingredient is within a range from 0.1 to 10,000 ppm. In the case of being formulated into oil solutions, aerosols, smoking agents, poison baits and the others, such formulations are used as itself without diluting it.

When the Compound of the present invention or the Composition A is used for controlling external parasites of livestock such as cows, horses, pigs, sheep, goats and chickens and small animals such as dogs, cats, rats and mice, the composition of the present invention may be applied to the animals by a known method in the veterinary field. Specifically, when systemic control is intended, the composition of the present invention is administered to the animals as a tablet, a mixture with feed or a suppository, or by injection (including intramuscular, subcutaneous, intravenous and intraperitoneal injections). On the other hand, when non-systemic control is intended, the composition of the present invention is applied to the animals by means of spraying of the oil solution or aqueous solution, pour-on or spot-on treatment, or washing of the animal with a shampoo formulation, or by putting a collar or ear tag made of the resin formulations to the animal. In the case of being administered to an animal body, the dose of the compound of the Compound of the present invention or the Composition A is usually within a range from 0.1 to 1,000 mg per 1 kg of an animal body weight.

Also, the composition of the Compound of the present invention or the Composition A may be used as an agent for controlling harmful arthropods in agricultural lands such as paddy fields, fields, turfs, and orchards. Examples of the plants include the followings.

corn (dent corn, flint corn, flour corn, popcorn, waxy corn, sweet corn, and field corn), rice (long grain rice, short grain rice, medium grain rice, japonica rice, tropical japonica rice, indica rice, javanica rice, paddy rice, upland rice, floating rice, direct-seeded rice, transplanted rice, and glutinous rice), wheat (bread wheat (hard wheat, soft wheat, medium wheat, red wheat, and white wheat), durum wheat, spelt wheat, and club wheat, winter wheat and spring wheat of them), barley (two-rowed barley (=barley for brewery), six-rowed barley, hull-less barley, and pearl barley, winter barley and spring barley of them), rye (winter rye and spring rye), triticale (winter triticale and spring triticale), oat (winter oat and spring oat), sorghum, cotton (upland cotton and Pima cotton), soybean (ripe seed harvest soybean, green soybeans, and early harvest soybeans, indeterminate type, determinate type, and semi-determinate type of them), peanut, buckwheat, beet (beets for sugar production, beets for feed, beets for root vegetable, beets for leaf vegetable, and beets for fuel), rapeseed (winter rapeseed and spring rapeseed), canola (winter canola and spring canola), sunflower (sunflowers for oil extraction, edible sunflowers, and sunflowers for ornamental purpose), sugar cane, tobacco, tea, mulberry, solanaceous vegetables (for example, eggplant, tomato, pimento, pepper, and potato), cucurbitaceous vegetables (for example, cucumber, pumpkin, zucchini, water melon, and melon), cruciferous vegetables (for example, Japanese radish, white turnip, horseradish, kohlrabi, Chinese cabbage, cabbage, leaf mustard, broccoli, and cauliflower), asteraceous vegetables (for example, burdock, crown daisy, artichoke, and lettuce), liliaceous vegetables (for example, welsh onion, onion, garlic, and asparagus), ammiaceous vegetables (for example, carrot, parsley, celery, and parsnip), chenopodiaceous vegetables (for example, spinach and Swiss chard), lamiaceous vegetables (for example, perilla, mint, and basil), strawberry, sweet potato, glutinous yam, eddoe, pomaceous fruits (for example, apple, pear, Japanese pear, Chinese white pear, Chinese quince, and quince), stone fleshy fruits (for example, peach, plum, nectarine, Japanese apricot (Prunus mume), cherry fruit, apricot, and prune), citrus fruits (for example, Citrus unshiu, orange, lemon, lime, and grapefruit), nuts (for example, chestnuts, walnuts, hazelnuts, almond, pistachio, cashew nuts, and macadamia nuts), berry fruits (for example, blueberry, cranberry, blackberry, and raspberry), grapes, Japanese persimmon, fig, olive, Japanese plum, banana, coffee, date palm, coconuts, ornamental plants, forest plants, turfs, grasses, and the others.

The above plants are not specifically limited as long as they are generally cultivated cultivars. The above plants also include plants which may be produced by natural breeding, plants which may be generated by mutation, F1 hybrid plants, and genetically modified crops. Examples of the genetically modified crops include plants which have resistance to HPPD (4-hydroxyphenylpyruvate dioxygenase enzyme) inhibitors such as isoxaflutole, ALS (acetolactate synthase) inhibitors such as imazethapyr and thifensulfuron-methyl, EPSP (5-enolpyruvylshikimate-3-phosphate synthase) inhibitors, glutamine synthetase inhibitors, PPO (protoporphyrinogen oxidase) inhibitors, or herbicide such as bromoxynil and dicamba; plants which can synthesize a selective toxin known in Bacillus spp. such as Bacillus thuringiensis or the like; and plants which can synthesize a gene fragment or the like which is partially identical to an endogenous gene derived from a harmful insect, and induce a gene silencing (RNAi; RNA interference) in the target harmful insect to achieve a specific insecticidal activity.

### EXAMPLES

Hereinafter, the present invention will be described in more detail with reference to production examples, formulation examples, test examples, and the like, but the present invention is not limited to these examples.

In the present specification, Me represents a methyl group, Et represents an ethyl group, Pr represents a propyl group, i-Pr represents an isopropyl group, Bu represents a butyl group, t-Bu represents a tert-butyl group, C₂F₅ represents a perfluoroethyl group, c-Pr represents a cyclopropyl group, c-Bu represents a cyclobutyl group, c-C₅H₉ represents a cyclopentyl group, c-C₆H₁₁ represents a cyclohexyl group, Ph represents a phenyl group, Py2 represents a 2-pyridyl group, Py3 represents a 3-pyridyl group, and Py4 represents a 4-pyridyl group. When c-Pr, Ph, Py2, Py3, and Py4 have a substituent, the substituent is described together with a substitution position before the symbol. For example, 1-CN-c-Pr represents a 1-cyanocyclopropyl group, 3-F-Ph represents a 3-fluorophenyl group, 3,4,5-(OCH₃)₃-Ph represents a 3,4,5-trimethoxyphenyl group, and 5-CF₃—Py2 represents a 5-(trifluoromethyl)-2-pyridyl group.

First, production examples of the present invention compound and production intermediates thereof will be shown.

### Reference Production Example 1

0.81 g of potassium carbonate was added to a mixture of 0.83 g of 5,6-dichloro-1-naphthol, 0.92 g of ethyl 5-(bromomethyl)isoxazole-3-carboxylate, and 5 mL of DMF, and the mixture was stirred at room temperature for 21 hours. Water was added to the resulting mixture, which was then extracted with methyl tert-butyl ether (hereinafter, referred to as MTBE). The resulting organic layer was washed with water and saturated saline, dried over anhydrous sodium sulfate, and concentrated under reduced pressure. The resulting residue was subjected to silica gel chromatography (hexane:ethyl acetate=3:1) to provide 0.95 g of Intermediate 1 represented by the following formula.

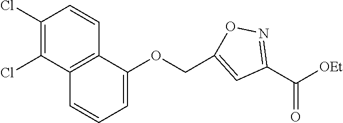 [SMILES: CCOC(=O)c1cc(COc2cccc3c(Cl)c(Cl)ccc23)on1]

Intermediate 1: ¹H-NMR (CDCl₃) δ: 8.14 (1H, d), 7.94 (1H, d), 7.54 (1H, t), 7.52 (1H, d), 6.94 (1H, d), 6.84 (1H, s), 5.40 (2H, s), 4.47 (2H, q), 1.43 (3H, t).

### Reference Production Example 1-1

The compound produced in accordance with Reference production example 1 and physical properties thereof are shown below.

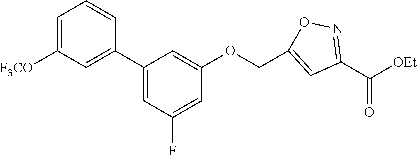 [SMILES: CCOC(=O)c1cc(COc2cc(F)cc(-c3cccc(OC(F)(F)F)c3)c2)on1]

Intermediate 2: ¹H-NMR (CDCl₃) δ: 7.51-7.42 (2H, m), 7.38 (1H, s), 7.26-7.20 (1H, m), 6.98-6.92 (2H, m), 6.81 (1H, s), 6.70 (1H, dt), 5.26 (2H, s), 4.46 (2H, q), 1.42 (3H, t).

### Reference Production Example 2

5 mL of a 1 mol/L aqueous sodium hydroxide solution was added to a mixture of 0.95 g of Intermediate 1 and 20 mL of THF, and the mixture was stirred for 18 hours. 10% Hydrochloric acid was added to the resulting mixture, and the mixture was extracted with MTBE. The resulting organic layer was dried over anhydrous sodium sulfate and concentrated under reduced pressure to provide 0.88 g of Intermediate 3 represented by the following formula.

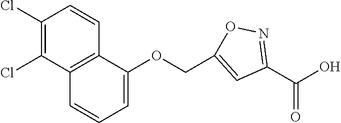 [SMILES: O=C(O)c1cc(COc2cccc3c(Cl)c(Cl)ccc23)on1]

Intermediate 3: ¹H-NMR (CDCl₃) δ: 8.14 (1H, d), 7.95 (1H, d), 7.56-7.51 (2H, m), 6.95 (1H, d), 6.90 (1H, s), 5.42 (2H, s).

### Reference Production Example 2-1

The compound produced in accordance with Reference production example 2 and physical properties thereof are shown below.

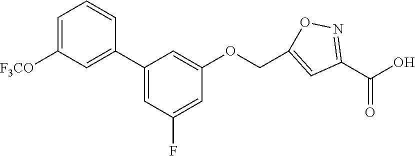 [SMILES: O=C(O)c1cc(COc2cc(F)cc(-c3cccc(OC(F)(F)F)c3)c2)on1]

Intermediate 4: ¹H-NMR (CDCl₃) δ: 7.51-7.44 (2H, m), 7.38 (1H, s), 7.26-7.21 (1H, m), 6.99-6.93 (2H, m), 6.85 (1H, s), 6.71 (1H, dt), 5.27 (2H, s).

### Reference Production Example 3

0.30 g of 1-(methylamino)cyclopentane methanamine was added to a mixture of 0.40 g of Intermediate 3, 0.45 g of 1-ethyl-3-(3-dimethylaminopropyl)carbodiimide hydrochloride, 0.02 g of 1-hydroxybenzotriazole, and 10 mL of chloroform, and the mixture was stirred at room temperature for 17 hours. The resulting mixture was concentrated under reduced pressure. The resulting residue was subjected to silica gel chromatography (chloroform methanol=10:1) to provide 0.40 g of Intermediate 5 represented by the following formula.

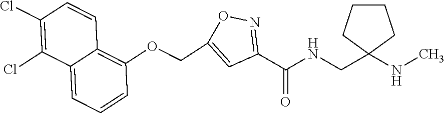 [SMILES: CNC1(CNC(=O)c2cc(COc3cccc4c(Cl)c(Cl)ccc34)on2)CCCC1]

Intermediate 5: ¹H-NMR (CDCl₃) δ: 8.15 (1H, d), 7.93 (1H, d), 7.53 (1H, t), 7.51 (1H, d), 7.44 (1H, s), 6.94 (1H, d), 6.88 (1H, s), 5.38 (2H, s), 3.40 (2H, d), 2.32 (3H, s), 1.76-1.34 (9H, m).

### Reference Production Example 3-1

The compound produced in accordance with Reference production example 3 and physical properties thereof are shown below.

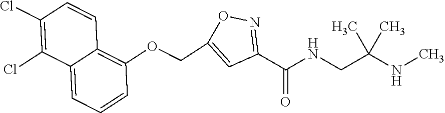 [SMILES: CNC(C)(C)CNC(=O)c1cc(COc2cccc3c(Cl)c(Cl)ccc23)on1]

Intermediate 6: ¹H-NMR (DMSO-D₆) δ: 8.52 (1H, s), 8.17 (1H, d), 7.83 (1H, d), 7.72-7.68 (2H, m), 7.34 (1H, d), 7.10 (1H, s), 5.61 (2H, s), 3.23 (2H, br), 2.28 (3H, s), 1.05 (6H, s).

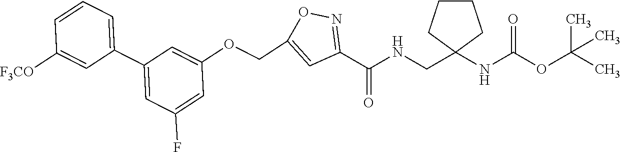 [SMILES: CC(C)(C)OC(=O)NC1(CNC(=O)c2cc(COc3cc(F)cc(-c4cccc(OC(F)(F)F)c4)c3)on2)CCCC1]

Intermediate 7: ¹H-NMR (CDCl₃) δ: 7.72 (1H, s), 7.50-7.43 (2H, m), 7.38 (1H, s), 7.26-7.20 (1H, m), 6.98-6.91 (2H, m), 6.82 (1H, s), 6.70 (1H, dt), 5.24 (2H, s), 4.65 (1H, s), 3.72 (2H, d), 1.89-1.64 (8H, m), 1.45 (9H, s).

### Reference Production Example 4

2 mL of trifluoroacetic acid was added to a mixture of 0.74 g of Intermediate 7 and 20 mL of chloroform, and the mixture was stirred at room temperature for 4 hours and 30 minutes. The resulting organic layer was dried over anhydrous sodium sulfate and concentrated under reduced pressure. The resulting residue was subjected to silica gel chromatography (chloroform:methanol=10:1) to provide 0.57 g of Intermediate 8 represented by the following formula.

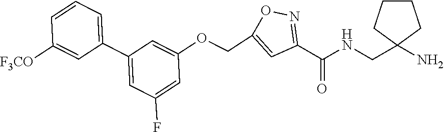 [SMILES: NC1(CNC(=O)c2cc(COc3cc(F)cc(-c4cccc(OC(F)(F)F)c4)c3)on2)CCCC1]

Intermediate 8: ¹H-NMR (CDCl₃) δ: 7.51-7.44 (2H, m), 7.38 (2H, s), 7.26-7.21 (1H, m), 6.99-6.92 (2H, m), 6.83 (1H, s), 6.70 (1H, dt), 5.24 (2H, s), 3.41 (2H, d), 1.86-1.61 (6H, m), 1.54-1.43 (2H, m), 1.42-1.24 (2H, m).

### Reference Production Example 5

0.31 g of 1,1′-bis(diphenylphosphino)ferrocenepalladium (II) dichloride was added to a mixture of 1.05 g of 5-bromo-2-difluoromethylbenzofuran, 1.62 g of bispinacol diboron, 1.25 g of potassium acetate, and 20 mL of dimethoxyethane, and the mixture was stirred at 80° C. for 3 hours and 30 minutes. After cooling to room temperature, water was added to the resulting mixture, which was then extracted with MTBE. The resulting organic layer was dried over anhydrous sodium sulfate and concentrated under reduced pressure. The resulting residue was subjected to silica gel chromatography (hexane:ethyl acetate=10:1) to provide 0.74 g of Intermediate 9 represented by the following formula.

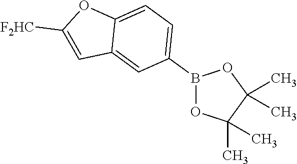 [SMILES: CC1(C)OB(c2ccc3oc(C(F)F)cc3c2)OC1(C)C]

Intermediate 9: ¹H-NMR (CDCl₃) δ: 8.14 (1H, s), 7.84 (1H, d), 7.53 (1H, d), 7.02 (1H, s), 6.75 (1H, t), 1.37 (12H, s).

### Reference Production Example 5-1

The compound produced in accordance with Reference production example 5 and physical properties thereof are shown below.

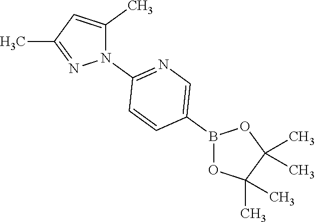

Intermediate 10: ¹H-NMR (CDCl₃) δ:8.75 (1H, dd), 8.12 (1H, dd), 7.84 (1H, dd), 5.99 (1H, s), 2.65 (3H, s), 2.30 (3H, s), 1.36 (12H, s).

### Reference Production Example 6

0.29 g of tetrakis(triphenylphosphine)palladium (0) was added to a mixture of 0.74 g of Intermediate 9, 0.59 g of ethyl 5-bromomethyl-isoxazole carboxylate, 1.04 g of potassium carbonate, 20 mL of toluene, and 4 mL of water, and the mixture was stirred under heat-reflux for 1 hour. After cooling to room temperature, water was added to the resulting mixture, which was then extracted with MTBE. The resulting organic layer was dried over anhydrous sodium sulfate and concentrated under reduced pressure. The resulting residue was subjected to silica gel chromatography (hexane:ethyl acetate=5:1) to provide 0.42 g of Intermediate 11 represented by the following formula.

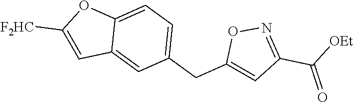 [SMILES: CCOC(=O)c1cc(Cc2ccc3oc(C(F)F)cc3c2)on1]

Intermediate 11: ¹H-NMR (CDCl₃) δ: 7.53 (1H, s), 7.52 (1H, d), 7.28 (1H, d), 7.00 (1H, s), 6.75 (1H, t), 6.33 (1H, s), 4.41 (2H, q), 4.24 (2H, s), 1.39 (3H, t).

### Reference Production Example 6-1

The compound produced in accordance with Reference production example 6 and physical properties thereof are shown below.

A compound represented by formula (A-1),

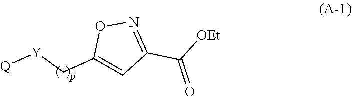

wherein (CH₂)ₚ—Y-Q is any one of [Table A-1].

TABLE A-1

| Intermediate | (CH₂)ₚ Y Q |
|---|---|
| 11 | CH₂-[4-(pylazol-1-yl)-Ph] |
| 12 | CH₂-[6-(pylazol-1-yl)-Py3] |
| 13 | CH₂-4-Ph-Ph |
| 14 | CH₂CH═CH-3,4-Me₂-Ph |
| 15 | CH₂-4-Py2-Ph |
| 16 | CH₂-[6-(3,5-Me₂-pylazol-1-yl)-Py3] |

Intermediate 11: ¹H-NMR (CDCl₃) δ: 7.92 (1H, d), 7.73 (1H, d), 7.68 (2H, d), 7.34 (2H, d), 6.48 (1H, t), 6.36 (1H, s), 4.42 (2H, q), 4.17 (2H, s), 1.40 (3H, t).

Intermediate 12: ¹H-NMR (CDCl₃) δ: 8.57 (1H, d), 8.33 (1H, d), 7.98 (1H, d), 7.74 (1H, d), 7.72 (1H, dd), 6.48 (1H, dd), 6.41 (1H, s), 4.43 (2H, q), 4.18 (2H, s), 1.40 (3H, t).

Intermediate 13: ¹H-NMR (CDCl₃) δ: 7.61-7.52 (4H, m), 7.48-7.41 (2H, m), 7.39-7.29 (3H, m), 6.39 (1H, s), 4.42 (2H, q), 4.18 (2H, s), 1.40 (3H, t).

Intermediate 14: ¹H-NMR (CDCl₃) δ: 7.15 (1H, s), 7.13-7.06 (2H, m), 6.51 (1H, d), 6.48 (1H, s), 6.21 (1H, dt), 4.43 (2H, q), 3.70 (2H, d), 2.26 (3H, s), 2.25 (3H, s), 1.41 (3H, t).

Intermediate 15: ¹H-NMR (CDCl₃) δ: 8.72-8.66 (1H, m), 7.98 (2H, d), 7.80-7.78 (2H, m), 7.37 (2H, d), 7.27-7.21 (1H, m), 6.36 (1H, s), 4.42 (2H, q), 4.19 (2H, s), 1.39 (3H, t).

Intermediate 16: ¹H-NMR (CDCl₃) δ: 8.32 (1H, d), 7.85 (1H, d), 7.67 (1H, dd), 6.39 (1H, s), 6.00 (1H, s), 4.42 (2H, q), 4.16 (2H, s), 2.63 (3H, s), 2.30 (3H, s), 1.40 (3H, t).

### Reference Production Example 7

The compound produced in accordance with Reference production example 2 and physical properties thereof are shown below.

A compound represented by formula (A-2),

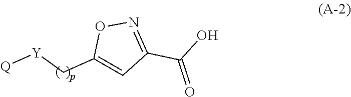

wherein (CH₂)ₚ—Y-Q is any one of [Table A-2].

TABLE A-2

| Intermediate | (CH₂)ₚ Y Q |
|---|---|
| 17 | CH₂-[4-(pylazol-1-yl)-Ph] |
| 18 | CH₂CH═CH-3,4-Me₂-Ph |
| 19 | CH₂-4-Py2-Ph |
| 20 | CH₂-[2-CHF₂-benzofuran-5-yl]] |
| 21 | CH₂O-3-F-5-C≡CH-Ph |

Intermediate 17: ¹H-NMR (CDCl₃) δ: 7.92 (1H, d), 7.76 (1H, d), 7.68 (2H, d), 7.35 (2H, d), 6.49 (1H, t), 6.41 (1H, s), 4.19 (2H, s).

Intermediate 18: ¹H-NMR (CDCl₃) δ: 7.16 (1H, s), 7.13-7.06 (2H, m), 6.54 (1H, s), 6.53 (1H, d), 6.21 (1H, dt), 3.73 (2H, d), 2.26 (3H, s), 2.25 (3H, s).

Intermediate 19: ¹H-NMR (DMSO-D₆) 5:8.68-8.63 (1H, m), 8.07 (2H, d), 7.93 (1H, dt), 7.88 (1H, td), 7.42 (2H, d), 7.34 (1H, ddd), 6.60 (1H, s), 4.28 (2H, s).

Intermediate 20: ¹H-NMR (CDCl₃) δ: 7.55-7.50 (2H, m), 7.28 (1H, dd), 7.00 (1H, s), 6.75 (1H, t), 6.39 (1H, s), 4.26 (2H, s).

Intermediate 21: ¹H-NMR (DMSO-D₆) δ: 7.10 (1H, dt), 7.05 (1H, s), 7.02-6.98 (1H, m), 6.98 (1H, s), 5.41 (2H, s), 4.36 (1H, s).

### Reference Production Example 8

The compound produced in accordance with Reference production example 3 and physical properties thereof are shown below.

A compound represented by formula (A-3),

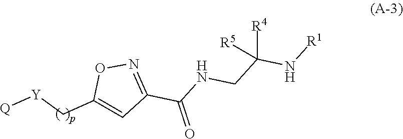

wherein the combination of R¹, R⁴, R⁵, and (CH₂)ₚ—Y-Q is any one of the combinations shown in [Table A-3].

TABLE A-3

| Intermediate | (CH₂)ₚ Y Q | R¹ | R⁴ | R⁵ |
|---|---|---|---|---|
| 22 | (CH₂)₃CH₃ | Me | Me | Me |

| | | | |
|---|---|---|---|
| 23 | CH₂-4-Me-Ph | Et | —(CH₂)₄— |
| 24 | CH₂-[4-(pylazol-1-yl)-Ph] | Me | —(CH₂)₄— |
| 25 | H | i-Pr | —(CH₂)₄— |
| 26 | CH₂-[4-(pylazol-1-yl)-Ph] | i-Pr | —(CH₂)₄— |
| 27 | CH₂O-[5,6-Cl₂-naphthalen-1-yl] | i-Pr | —(CH₂)₄— |
| 28 | CH₂-4-Me-Ph | i-Pr | —(CH₂)₄— |
| 29 | (CH₂)₃Ph | i-Pr | —(CH₂)₄— |
| 30 | CH₂CH═CH-3,4-Me₂-Ph | i-Pr | —(CH₂)₄— |
| 31 | CH₂CH (CH₃)₂ | i-Pr | —(CH₂)₄— |
| 32 | CH₂Ph | i-Pr | —(CH₂)₄— |
| 33 | CH₂-4-Py2-Ph | i-Pr | —(CH₂)₄— |
| 34 | CH₂-[2-CHF₂-benzofuran-5-yl] | i-Pr | —(CH₂)₄— |
| 35 | CH₂O-[3-F-5-(3-OCF₃-Ph)-Ph] | i-Pr | —(CH₂)₄— |
| 36 | CH₂O-3-F-5-C≡CH-Ph | i-Pr | —(CH₂)₄— |
| 37 | CH₂OCH₂-3,4-Cl₂-Ph | i-Pr | —(CH₂)₄— |

Intermediate 22: ¹H-NMR (CDCl₃) δ: 8.45-8.36 (1H, m), 6.42 (1H, s), 3.35 (2H, d), 2.78 (2H, t), 2.16 (3H, s), 1.74-1.65 (2H, m), 1.45-1.35 (2H, m), 1.02 (6H, s), 0.94 (3H, t).

Intermediate 23: ¹H-NMR (CDCl₃) δ: 7.46-7.34 (1H, m), 7.14 (4H, s), 6.36 (1H, s), 4.07 (2H, s), 3.35 (2H, d), 2.53 (2H, q), 2.33 (3H, s), 1.78-1.46 (9H, m), 1.08 (3H, t).

Intermediate 24: ¹H-NMR (CDCl₃) δ: 7.91 (1H, d), 7.72 (1H, d), 7.69-7.55 (3H, m), 7.32 (2H, d), 6.47 (1H, t), 6.43 (1H, s), 4.13 (2H, s), 3.47 (2H, d), 2.40 (3H, s), 1.80-1.55 (8H, m).

Intermediate 25: ¹H-NMR (CDCl₃) δ: 8.47 (1H, d), 7.68-7.53 (1H, m), 6.82 (1H, d), 3.36 (2H, d), 2.96-2.84 (1H, m), 1.76-1.53 (8H, m), 1.08 (6H, d).

Intermediate 26: ¹H-NMR (CDCl₃) δ: 7.91 (1H, d), 7.73 (1H, d), 7.67 (2H, d), 7.56-7.48 (1H, m), 7.34 (2H, d), 6.47 (1H, dd), 6.42 (1H, s), 4.15 (2H, s), 3.33 (2H, d), 2.93-2.83 (1H, m), 1.74-1.45 (9H, m), 1.06 (6H, d).

Intermediate 27: ¹H-NMR (CDCl₃) δ: 8.13 (1H, d), 7.91 (1H, d), 7.62 (1H, s), 7.53-7.49 (2H, m), 6.93 (1H, d), 6.88 (1H, s), 5.37 (2H, s), 3.36 (2H, d), 2.93-2.87 (1H, m), 1.72-1.56 (8H, m), 1.09 (3H, s), 1.07 (3H, s).

Intermediate 28: ¹H-NMR (CDCl₃) δ: 7.50 (1H, s), 7.14 (4H, s), 6.36 (1H, s), 4.06 (2H, s), 3.33 (2H, d), 2.91-2.85 (1H, m), 2.33 (3H, s), 1.71-1.53 (8H, m), 1.07 (3H, s), 1.05 (3H, s).

Intermediate 29: ¹H-NMR (CDCl₃) δ: 7.52 (1H, s), 7.30-7.28 (2H, m), 7.24-7.17 (3H, m), 6.45 (1H, s), 3.35 (2H, d), 2.93-2.86 (1H, m), 2.80 (2H, t), 2.69 (2H, t), 2.09-2.01 (2H, m), 1.71-1.55 (8H, m), 1.08 (3H, s), 1.06 (3H, s).

Intermediate 30: ¹H-NMR (CDCl₃) δ: 7.52 (1H, s), 7.14 (1H, s), 7.11-7.06 (2H, m), 6.52-6.48 (2H, m), 6.24-6.16 (1H, m), 3.68 (2H, d), 3.35 (2H, d), 2.92-2.86 (1H, m), 2.25 (6H, s), 1.70-1.56 (8H, m), 1.08 (3H, s), 1.06 (3H, s).

Intermediate 31: ¹H-NMR (CDCl₃) δ: 7.53 (1H, s), 6.43 (1H, s), 3.35 (2H, d), 2.93-2.87 (1H, m), 2.67 (2H, d), 2.11-2.00 (1H, m), 1.71-1.55 (8H, m), 1.08 (3H, s), 1.06 (3H, s), 0.98 (3H, s), 0.96 (3H, s).

Intermediate 32: ¹H-NMR (CDCl₃) δ: 7.51 (1H, s), 7.35-7.29 (3H, m), 7.27-7.24 (2H, m), 6.38 (1H, t), 4.11 (2H, s), 3.33 (2H, d), 2.92-2.85 (1H, m), 1.70-1.53 (8H, m), 1.07 (3H, s), 1.05 (3H, s).

Intermediate 33: ¹H-NMR (CDCl₃) δ: 8.70-8.68 (1H, m), 7.98-7.95 (2H, m), 7.78-7.70 (2H, m), 7.52 (1H, s), 7.38-7.35 (2H, m), 7.25-7.21 (1H, m), 6.43 (1H, t), 4.17 (2H, s), 3.33 (2H, d), 2.92-2.85 (1H, m), 1.70-1.53 (8H, m), 1.07 (3H, s), 1.05 (3H, s).

Intermediate 34: ¹H-NMR (CDCl₃) δ: 7.52-7.49 (3H, m), 7.29-7.27 (1H, m), 6.99 (1H, s), 6.75 (1H, t), 6.39 (1H, s), 4.21 (2H, s), 3.33 (2H, d), 2.91-2.85 (1H, m), 1.69-1.54 (8H, m), 1.07 (3H, s), 1.05 (3H, s).

Intermediate 35: ¹H-NMR (CDCl₃) δ: 7.62 (1H, s), 7.48-7.45 (2H, m), 7.38 (1H, s), 7.24-7.22 (1H, m), 6.95-6.92 (2H, m), 6.82 (1H, s), 6.72-6.68 (1H, m), 5.24 (2H, s), 3.36 (2H, d), 2.93-2.87 (1H, m), 1.71-1.56 (8H, m), 1.09 (3H, s), 1.07 (3H, s).

Intermediate 36: ¹H-NMR (CDCl₃) δ: 7.59 (1H, s), 6.88-6.86 (2H, m), 6.79 (1H, s), 6.71-6.67 (1H, m), 5.17 (2H, s), 3.35 (2H, d), 3.11 (1H, s), 2.93-2.86 (1H, m), 1.72-1.56 (8H, m), 1.08 (3H, s), 1.07 (3H, s).

Intermediate 37: ¹H-NMR (CDCl₃) δ: 7.58 (1H, s), 7.45-7.42 (2H, m), 7.19-7.16 (1H, m), 6.73 (1H, s), 4.67 (2H, s), 4.55 (2H, s), 3.35 (2H, d), 2.93-2.87 (1H, m), 1.71-1.55 (8H, m), 1.08 (3H, s), 1.07 (3H, s).

### Reference Production Example 9

To 0.56 g of Intermediate 12 was added 0.59 g of 1-[(1-methylethyl)amino]cyclopentane methanamine, and the mixture was stirred at 130° C. for 2 hours. After cooling to room temperature, the resulting mixture was concentrated under reduced pressure. The resulting residue was subjected to silica gel chromatography (chloroform:methanol=10:1) to provide 0.58 g of Intermediate 38 represented by the following formula.

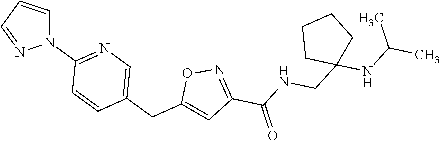 [SMILES: CC(C)NC1(CNC(=O)c2cc(Cc3ccc(-n4cccn4)nc3)on2)CCCC1]

Intermediate 38: ¹H-NMR (CDCl₃) δ: 8.54 (1H, d), 8.33 (1H, d), 7.97 (1H, d), 7.74 (1H, d), 7.72 (1H, dd), 7.58-7.50 (1H, m), 6.47 (1H, s), 6.46 (1H, s), 4.16 (2H, s), 3.33 (2H, d), 2.94-2.83 (1H, m), 1.74-1.48 (9H, m), 1.07 (6H, d).

### Reference Production Example 9-1

The compound produced in accordance with Reference production example 9 and physical properties thereof are shown below.

A compound represented by formula (A-4),

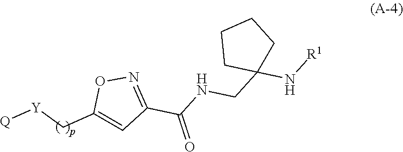

wherein the combination of R¹ and (CH₂)ₚ—Y-Q is any one of the combinations shown in [Table A-4].

TABLE A-4

| Intermediate | (CH₂)ₚ Y Q | R1 |
|---|---|---|
| 39 | CH₂-4-Ph-Ph | i-Pr |
| 40 | CH₂-4-OCF₃-Ph | i-Pr |
| 41 | CH₂-[6-(3,5-Me₂-pylazol-1-yl)-Py3] | i-Pr |
| 42 | CH₂-[6-(3,5-Me₂-pylazol-1-yl)-Py3] | Et |
| 43 | CH₂-[6-(3,5-Me₂-pylazol-1-yl)-Py3] | Me |

Intermediate 39: ¹H-NMR (CDCl₃) δ: 7.59-7.50 (5H, m), 7.47-7.41 (2H, m), 7.37-7.30 (3H, m), 6.43 (1H, s), 4.15 (2H, s), 3.33 (2H, d), 2.92-2.85 (1H, m), 1.70-1.53 (8H, m), 1.07 (3H, s), 1.05 (3H, s).

Intermediate 40: ¹H-NMR (CDCl₃) δ: 7.53 (1H, s), 7.28 (2H, d), 7.19 (2H, d), 6.41 (1H, s), 4.12 (2H, s), 3.33 (2H, d), 2.92-2.85 (1H, m), 1.70-1.55 (8H, m), 1.07 (3H, s), 1.06 (3H, s).

Intermediate 41: ¹H-NMR (CDCl₃) δ: 8.33 (1H, d), 7.83 (1H, d), 7.67 (1H, dd), 7.57-7.49 (1H, m), 6.45 (1H, s), 5.99 (1H, s), 4.13 (2H, s), 3.33 (2H, d), 2.93-2.83 (1H, m), 2.62 (3H, s), 2.29 (3H, s), 1.75-1.52 (9H, m), 1.06 (6H, d).

Intermediate 42: ¹H-NMR (CDCl₃) δ: 8.32 (1H, s), 7.83 (1H, d), 7.66 (1H, d), 7.49-7.39 (1H, m), 6.45 (1H, s), 5.99 (1H, s), 4.14 (2H, s), 3.36 (2H, d), 2.62 (3H, s), 2.54 (2H, q), 2.29 (3H, s), 1.76-1.47 (9H, m), 1.09 (3H, t).

Intermediate 43: ¹H-NMR (CDCl₃) δ: 8.32 (1H, d), 7.83 (1H, d), 7.66 (1H, dd), 7.40-7.30 (1H, m), 6.45 (1H, s), 5.99 (1H, s), 4.14 (2H, s), 3.38 (2H, d), 2.62 (3H, s), 2.30 (3H, s), 2.29 (3H, s), 1.74-1.40 (9H, m).

### Production Example 1

0.59 g of paraformaldehyde was added to a mixture of 0.50 g of 5-butyl-N-[2-methyl-2-(methylamino)propyl]-3-isoxazole carboxamide produced by the method described in WO 2016/017467 A and 22 mL of THF, and the mixture was stirred under heat-reflux for 2 hours. The resulting mixture was filtered and concentrated under reduced pressure. The resulting residue was subjected to silica gel chromatography (hexane:ethyl acetate=1:1) to provide 0.36 g of Present invention compound 1 represented by the following formula.

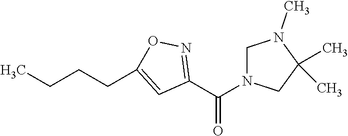 [SMILES: CCCCc1cc(C(=O)N2CN(C)C(C)(C)C2)no1]

Compound of the present invention 1: ¹H-NMR (CDCl₃) δ: 6.43 (1H, d), 4.60 (1H, s), 4.40 (1H, s), 3.81 (1H, s), 3.54 (1H, s), 2.79 (2H, t), 2.29 (3H, d), 1.72-1.66 (2H, m), 1.45-1.36 (2H, m), 1.14 (3H, s), 1.12 (3H, s), 0.95 (3H, t).

### Production Example 1-1

The compound produced in accordance with Production example 1 and physical properties thereof are shown below. A compound represented by formula (B-1),

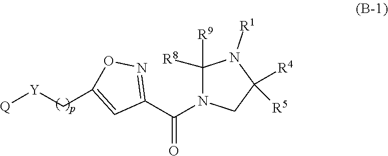

wherein the combination of R¹, R⁴, R⁵, R³, R⁹ and (CH₂)ₚ—Y-Q is any one of the combinations shown in [Table B-1], [Table B-2], [Table B-3], and [Table B-4].

TABLE B-1

| Compound | | | | | | |
|---|---|---|---|---|---|---|
| of the | | | | | | |
| present | | | | | | |
| invention | (CH₂)ₚ Y Q | R¹ | R⁴ | R⁵ | R⁸ | R⁹ |
| 2 | (CH₂)₃CH₃ | H | Me | Me | H | H |
| 3 | (CH₂)₃CH₃ | Me | Me | Me | i-Pr | H |
| 4 | (CH₂)₃CH₃ | H | Me | Me | Me | Me |
| 5 | (CH₂)₃CH₃ | H | Me | Me | t-Bu | H |
| 6 | CH₂(c-Pr) | H | Me | Me | t-Bu | H |
| 7 | CH₂Ph | H | Me | Me | t-Bu | H |
| 8 | CH₂-3-F-Ph | H | Me | Me | t-Bu | H |
| 9 | CH₂OPh | H | Me | Me | t-Bu | H |
| 10 | (CH₂)₇CH₃ | H | Me | Me | t-Bu | H |
| 11 | (CH₂)₂CF₃ | H | Me | Me | t-Bu | H |
| 12 | CH₂OCH₂-[1,3-benzodioxol-5-yl] | H | Me | Me | t-Bu | H |
| 13 | (CH₂)₂(c-C₅H₉) | H | Me | Me | t-Bu | H |
| 14 | CH₂O-[5,6-Cl₂-naphthalen-1-yl] | Me | Me | Me | H | H |

| | | | | | |
|---|---|---|---|---|---|
| 15 | CH₂-[5,6-Me₂-benzofuran-2-yl] | H | —(CH₂)₄— | Me | Me |
| 16 | CH₂-[5,6-Me₂-benzofuran-2-yl] | Et | —(CH₂)₄— | H | H |
| 17 | CH₂-[5,6-Me₂-benzofuran-2-yl] | Et | —(CH₂)₄— | C(O)OEt | H |
| 18 | CH₂-[5,6-Me₂-benzofuran-2-yl] | i-Pr | —(CH₂)₄— | Me | H |
| 19 | CH₂-[5,6-Me₂-benzofuran-2-yl] | Et | —(CH₂)₄— | Me | H |
| 20 | CH₂-[4,6-Me₂-5-Cl-benzofuran-2- | i-Pr | —(CH₂)₄— | Me | H |
| | yl] | | | | |
| 21 | CH₂-[4,6-Me₂-5-Cl-benzofuran-2- | i-Pr | —(CH₂)₄— | H | H |
| | yl] | | | | |
| 22 | CH₂-[5,6-Me₂-benzofuran-2-yl] | i-Pr | —(CH₂)₄— | H | H |
| 23 | CH₂-[5,6-Me₂-benzofuran-2-yl] | i-Pr | —(CH₂)₄— | c-Pr | H |
| 24 | CH₂-[4,6-Me₂-5-Cl-benzofuran-2- | i-Pr | —(CH₂)₄— | c-Pr | H |
| | yl] | | | | |
| 25 | CH₂-[5,6-Me₂-benzofuran-2-yl] | Et | —(CH₂)₄— | c-Pr | H |
| 26 | CH₂-[4,6-Me₂-5-Cl-benzofuran-2- | Et | —(CH₂)₄— | c-Pr | H |
| | yl] | | | | |
| 27 | CH₂-[4,6-Me₂-5-Cl-benzofuran-2- | Et | —(CH₂)₄— | H | H |
| | yl] | | | | |
| 28 | CH₂-[5,6-Me₂-benzofuran-2-yl] | i-Pr | —(CH₂)₄— | Et | H |

TABLE B-2

| | | | | | | |
|---|---|---|---|---|---|---|
| Compound | | | | | | |
| of the | | | | | | |
| present | | | | | | |
| invention | (CH₂)ₚ Y Q | R¹ | R⁴ | R⁵ | R⁸ | R⁹ |

| | | | | | |
|---|---|---|---|---|---|
| 29 | CH₂-[4,6-Me₂-5-Cl-benzofuran-2- | Et | —(CH₂)₄— | i-Pr | H |
| 30 | CH₂-[5,6-Me₂-benzofuran-2-yl] | i-Pr | —(CH₂)₄— | CH═CHCH₃ | H |
| 31 | CH₂-[5,6-Me₂-benzofuran-2-yl] | Et | —(CH₂)₄— | i-Pr | H |
| 32 | CH₂-[5,6-Me₂-benzofuran-2-yl] | i-Pr | —(CH₂)₄— | (CH₂) 2CH₃ | H |
| 33 | CH₂-[5,6-Me₂-benzofuran-2-yl] | i-Pr | —(CH₂)₄— | (CH₂)₃CH₃ | H |
| 34 | CH₂-[benzofuran-2-yl] | i-Pr | —(CH₂)₄— | Me | H |
| 35 | CH₂-[5-Me-benzofuran-2-yl] | i-Pr | —(CH₂)₄— | Me | H |
| 36 | CH₂-[5-Et-benzofuran-2-yl] | i-Pr | —(CH₂)₄— | Me | H |
| 37 | CH₂-[5-i-Pr-benzofuran-2-yl] | i-Pr | —(CH₂)₄— | Me | H |
| 38 | CH₂-[6-F-benzofuran-2-yl] | i-Pr | —(CH₂)₄— | Me | H |
| 39 | CH₂-[5-Cl-benzofuran-2-yl] | i-Pr | —(CH₂)₄— | Me | H |
| 40 | CH₂-[6-OMe-benzofuran-2-y]] | i-Pr | —(CH₂)₄— | Me | H |
| 41 | CH₂-[5-Me-6-OMe-benzofuran-2- | i-Pr | —(CH₂)₄— | Me | H |
| | yl] | | | | |
| 42 | CH₂-[6-Me-benzofuran-2-yl] | i-Pr | —(CH₂)₄— | Me | H |
| 43 | CH₂-[5,6-Me₂-benzothiophen-2- | i-Pr | —(CH₂)₄— | Me | H |
| | yl] | | | | |
| 44 | CH₂-[4,5-Me₂-benzothiophen-2- | i-Pr | —(CH₂)₄— | \|Me | H |
| | yl] | | | | |
| 45 | CH₂-[4,5,6-Me3-benzofuran-2-yl] | i-Pr | —(CH₂)₄— | Me | H |
| 46 | CH₂-[4,6-Me₂-benzofuran-2-yl] | i-Pr | —(CH₂)₄— | Me | H |
| 47 | CH₂-[5-C1-6-Me-benzofuran-2-yl] | i-Pr | —(CH₂)₄— | Me | H |
| 48 | CH₂-[5-F-benzofuran-2-yl] | i-Pr | —(CH₂)₄— | Me | H |
| 49 | CH₂-[6-Me-benzofuran-2-y]] | H | —(CH₂)₄— | t-Bu | H |
| 50 | CH₂O-[3-F-5-(3-OCF₃-Ph)-Ph] | H | —(CH₂)₄— | t-Bu | H |
| 51 | CH₂-[5,6-Me₂-benzofuran-2-yl] | H | —(CH₂)₄— | t-Bu | H |
| 52 | CH₂-[4,6-Me₂-5-Cl-benzofuran-2- | i-Pr | —(CH₂)₄— | i-Pr | H |
| | yl] | | | | |
| 53 | CH₂-[4,6-Me₂-5-Cl-benzofuran-2- | i-Pr | —(CH₂)₄— | CH₂OCH₃ | H |
| | yl] | | | | |
| 54 | CH₂-[4,6-Me₂-5-Cl-benzofuran-2- | i-Pr | —(CH₂)₄— | CH₂Ph | H |
| | yl] | | | | |
| 55 | CH₂O-[5,6-Cl₂-naphthalen-1-yl] | Me | —(CH₂)₄— | H | H |

TABLE B-3

| | | | | | | |
|---|---|---|---|---|---|---|
| Compound | | | | | | |
| of the | | | | | | |
| present | | | | | | |
| invention | (CH₂)ₚ Y Q | R¹ | R⁴ | R⁵ | R⁸ | R⁹ |

| | | | | | |
|---|---|---|---|---|---|
| 56 | (CH₂)₃CH₃ | Me | —(CH₂)₄— | i-Pr | H |
| 57 | CH₂-4-Me-Ph | Et | —(CH₂)₄— | i-Pr | H |
| 58 | CH₂-[4,6-Me₂-5-Cl-benzofuran-2- | H | —(CH₂)₄— | t-Bu | H |
| 59 | CH₂-[4,6-Me₂-5-Cl-benzofuran-2- | H | —(CH₂)₄— | H | H |
| | yl] | | | | |
| 60 | CH₂-[4,6-Me₂-5-Cl-benzofuran-2- | Me | —(CH₂)₄— | i-Pr | H |
| | yl] | | | | |
| 61 | CH₂-[4,6-Me₂-5-Cl-benzofuran-2- | Me | —(CH₂)₄— | H | H |
| | yl] | | | | |
| 62 | CH₂O-[5,6-Cl₂-naphthalen-1-yl] | Me | —(CH₂)₄— | i-Pr | H |
| 63 | CH₂-[4-(pylazol-1-yl)-Ph] | Me | —(CH₂)₄— | i-Pr | H |
| 64 | H | i-Pr | —(CH₂)₄— | Me | H |
| 65 | CH₂-[4-(pylazol-1-yl)-Ph] | i-Pr | —(CH₂)₄— | Me | H |
| 66 | CH₂-[6-(pylazol-1-yl)-Py3] | i-Pr | —(CH₂)₄— | Me | H |
| 67 | CH₂O-[5,6-Cl₂-naphthalen-1-yl] | i-Pr | —(CH₂)₄— | Me | H |
| 68 | CH₂-4-Ph-Ph | i-Pr | —(CH₂)₄— | Me | H |
| 69 | CH₂-4-OCF₃-Ph | i-Pr | —(CH₂)₄— | Me | H |
| 70 | CH₂-4-Me-Ph | i-Pr | —(CH₂)₄— | Me | H |
| 71 | CH₂-[5,6-Me₂-benzofuran-2-yl] | i-Pr | —(CH₂)₄— | CH₂CH(CH₃)₂ | H |
| 72 | CH₂O-4-OCF₃-Ph | i-Pr | —(CH₂)₄— | Me | H |
| 73 | CH₂-[5,6-Me₂-benzofuran-2-yl] | CH₂C≡CH | —(CH₂)₄— | i-Pr | H |
| 74 | (CH₂)₃Ph | i-Pr | —(CH₂)₄— | Me | H |
| 75 | CH₂CH═CH-3,4-Me₂-Ph | i-Pr | —(CH₂)₄— | Me | H |
| 76 | CH₂CH (CH₃)₂ | i-Pr | —(CH₂)₄— | Me | H |
| 77 | CH₂Ph | i-Pr | —(CH₂)₄— | Me | H |
| 78 | CH₂-4-Py2-Ph | i-Pr | —(CH₂)₄— | Me | H |
| 79 | CH₂-[2-CHF2-benzofuran-5-yl] | i-Pr | —(CH₂)₄— | Me | H |
| 80 | CH₂-[5,6-Me₂-benzofuran-2-yl] | i-Pr | —(CH₂)₄— | C(O)OEt | H |
| 81 | CH₂-[4,6-Me₂-5-Cl-benzofuran-2- | i-Pr | —(CH₂)₄— | C(O)OEt | H |
| | yl] | | | | |
| 82 | CH₂O-[3-F-5-(3-OCF₃-Ph)-Ph] | i-Pr | —(CH₂)₄— | Me | H |

TABLE B-4

| Compound | | | | | | |
|---|---|---|---|---|---|---|
| of the | | | | | | |
| present | | | | | | |
| invention | (CH₂)ₚ Y Q | R¹ | R⁴ | R⁵ | R⁸ | R⁹ |
| 83 | CH₂O-3-F-5-C≡CH-Ph | i-Pr | —(CH₂)₄— | | Me | H |
| 84 | CH₂-[5,6-Me₂-benzofuran-2-yl] | CH₂Ph | —(CH₂)₄— | | i-Pr | H |
| 85 | CH₂OCH₂-3,4-Cl₂-Ph | i-Pr | —(CH₂)₄— | | Me | H |
| 86 | CH₂-[5,6-Me₂-benzofuran-2-yl] | CH₂Ph | —(CH₂)₄— | | Me | H |
| 87 | CH₂-[5,6-Me₂-benzofuran-2-yl] | CH₂Ph | —(CH₂)₄— | | H | H |
| 88 | CH₂-[5,6-Me₂-benzofuran-2-yl] | CH₂Ph | —(CH₂)₄— | | C(O)OEt | H |
| 89 | CH₂-[5,6-Me₂-benzofuran-2-yl] | Et | —(CH₂)₄— | | furan-2- | H |
| 90 | CH₂-[5,6-Me₂-benzofuran-2-yl] | Et | —(CH₂)₄— | | 4-OMe-Ph | H |
| 91 | CH₂-[5,6-Me₂-benzofuran-2-yl] | Et | —(CH₂)₄— | | Ph | H |
| 92 | CH₂-[5,6-Me₂-benzofuran-2-yl] | Et | —(CH₂)₄— | | furan-3- | H |
| | | | | | yl | |
| 93 | CH₂-[6-(3,5-Me₂-pylazol-1-yl)- | i-Pr | —(CH₂)₄— | | Me | H |
| | Py3] | | | | | |
| 94 | CH₂-[5,6-Me₂-benzofuran-2-yl] | Et | —(CH₂)₄— | | 4-CF₃-Ph | H |
| 95 | CH₂-[5,6-Me₂-benzofuran-2-yl] | Et | —(CH₂)₄— | | thiophen- | H |
| | | | | | 3-yl | |
| 96 | CH₂-[5,6-Me₂-benzofuran-2-yl] | i-Pr | —(CH₂)₄— | | furan-2- | H |
| | | | | | yl | |
| 97 | CH₂-[6-(3,5-Me₂-pylazol-1-yl)- | Et | —(CH₂)₄— | | Ph | H |
| | Py3] | | | | | |
| 98 | CH₂-[5,6-Me₂-benzofuran-2-yl] | Et | —(CH₂)₄— | | t-Bu | H |
| 99 | CH₂-[5,6-Me₂-benzofuran-2-yl] | Me | —(CH₂)₄— | | t-Bu | H |
| 100 | CH₂-[5,6-Me₂-benzofuran-2-yl] | Me | Me | Me | t-Bu | H |

| | | | | | |
|---|---|---|---|---|---|
| 101 | CH₂-[5,6-Me₂-benzofuran-2-yl] | Me | —(CH₂)₄— | C(O)OEt | H |
| 102 | CH₂-[5-C1-6-Me-benzofuran-2-yl] | Me | —(CH₂)₄— | C(O)OEt | H |
| 103 | CH₂-[5-C1-6-Me-benzofuran-2-y]] | Me | —(CH₂)₄— | t-Bu | H |
| 104 | CH₂-[5,6-Me₂-benzofuran-2-yl] | CH₂C═CH | —(CH₂)₄— | t-Bu | H |
| 105 | CH₂-[5,6-Me₂-benzofuran-2-yl] | CH₂C═CH | —(CH₂)₄— | C(O)OEt | H |
| 106 | CH₂-[6-(3,5-Me₂-pylazol-1-yl)- | Me | —(CH₂)₄— | t-Bu | H |
| | Py3] | | | | |
| 107 | CH₂-[5,6-Me₂-benzofuran-2-yl] | Me | —(CH₂)₄— | i-Pr | H |
| 108 | CH₂-[5,6-Me₂-benzofuran-2-yl] | Me | —(CH₂)₄— | H | H |

Compound of the present invention 2: ¹H-NMR (CDCl₃) δ: 6.43 (1H, d), 4.86 (1H, s), 4.67 (1H, s), 3.65 (1H, s), 3.41 (1H, s), 2.79 (2H, t), 1.74-1.66 (2H, m), 1.45-1.36 (2H, m), 1.30 (3H, s), 1.28 (3H, s), 0.95 (3H, t).

Compound of the present invention 3: ¹H-NMR (CDCl₃) δ: 6.36 (1H, d), 4.50 (1H, d), 3.95 (1H, d), 3.40 (1H, d), 2.78 (2H, t), 2.29 (3H, s), 2.27-2.20 (1H, m), 1.74-1.66 (2H, m), 1.46-1.36 (2H, m), 1.14 (3H, s), 1.01 (3H, d), 0.99 (3H, d), 0.95 (3H, t), 0.90 (3H, s).

Compound of the present invention 4: ¹H-NMR (CDCl₃) δ: 6.38 (1H, s), 3.81 (2H, s), 2.78 (2H, t), 1.73 (6H, s), 1.72-1.64 (2H, m), 1.46-1.35 (2H, m), 1.31 (6H, s), 1.26 (1H, s), 0.95 (3H, t).

Compound of the present invention 5: ¹H-NMR (CDCl₃) δ: 7.53 (1H, s), 7.30 (1H, s), 6.43 (1H, s), 3.43 (2H, d), 2.78 (2H, t), 1.73-1.65 (2H, m), 1.46-1.35 (2H, m), 1.15 (6H, s), 1.06 (9H, s), 0.94 (3H, t).

Compound of the present invention 6: ¹H-NMR (CDCl₃) δ: 7.53 (1H, s), 7.31 (1H, s), 6.55 (1H, t), 3.42 (2H, d), 2.68 (2H, d), 1.15 (6H, s), 1.06 (10H, s), 0.63-0.59 (2H, m), 0.27-0.23 (2H, m).

Compound of the present invention 7: ¹H-NMR (CDCl₃) δ: 7.52 (1H, s), 7.36-7.24 (6H, m), 6.38 (1H, t), 4.10 (2H, s), 3.40 (2H, d), 1.13 (6H, s), 1.05 (9H, s).

Compound of the present invention 8: ¹H-NMR (CDCl₃) δ: 7.52 (1H, s), 7.33-7.29 (2H, m), 7.04-7.02 (1H, m), 7.01-6.94 (2H, m), 6.42 (1H, t), 4.11 (2H, s), 3.40 (2H, d), 1.14 (6H, s), 1.05 (9H, s).

Compound of the present invention 9: ¹H-NMR (CDCl₃) δ: 7.53 (1H, s), 7.33-7.29 (3H, m), 6.97-6.94 (3H, m), 6.78 (1H, t), 5.18 (2H, s), 3.42 (2H, d), 1.15 (6H, s), 1.06 (9H, s).

Compound of the present invention 10: ¹H-NMR (CDCl₃) δ: 7.53 (1H, s), 7.30 (1H, s), 6.42 (1H, t), 3.41 (2H, d), 2.77 (2H, t), 1.70 (2H, t), 1.32-1.26 (10H, m), 1.15 (6H, s), 1.06 (9H, s), 0.88 (3H, t).

Compound of the present invention 11: ¹H-NMR (CDCl₃) δ: 7.53 (1H, s), 7.34 (1H, s), 6.53 (1H, t), 3.42 (2H, d), 3.10-3.06 (2H, m), 2.60-2.48 (2H, m), 1.15 (6H, s), 1.06 (9H, s).

Compound of the present invention 12: ¹H-NMR (CDCl₃) δ: 7.53 (1H, s), 7.35 (1H, s), 6.85 (1H, s), 6.79 (2H, m), 6.70 (1H, s), 5.97 (2H, s), 4.64 (2H, s), 4.50 (2H, s), 3.42 (2H, d), 1.15 (6H, s), 1.06 (9H, s).

Compound of the present invention 13: ¹H-NMR (CDCl₃) δ: 7.53 (1H, s), 7.30 (1H, s), 6.43 (1H, t), 3.41 (2H, d), 2.79 (2H, t), 1.82-1.50 (11H, m), 1.15 (6H, s), 1.06 (9H, s).

Compound of the present invention 14: 8.14 (1H, d), 7.92 (1H, d), 7.54-7.49 (2H, m), 6.95 (1H, d), 6.88 (1H, d), 5.38 (2H, s), 4.63 (1H, s), 4.41 (1H, s), 3.83 (1H, s), 3.56 (1H, s), 2.30 (3H, d), 1.14 (6H, d).

Compound of the present invention 15: ¹H-NMR (CDCl₃) δ: 7.26 (1H, s), 7.21 (1H, s), 6.53 (1H, s), 6.48 (1H, s), 4.27 (2H, s), 3.82 (2H, s), 2.34 (3H, s), 2.32 (3H, s), 1.87-1.56 (12H, m), 1.36-1.19 (3H, m).

Compound of the present invention 16: ¹H-NMR (CDCl₃) δ: 7.26 (1H, s), 7.21 (1H, s), 6.58 (1H, s), 6.48 (1H, s), 4.61 (1H, s), 4.41 (1H, s), 4.27 (2H, s), 3.82 (1H, s), 3.57 (1H, s), 2.54 (2H, q), 2.34 (3H, s), 2.32 (3H, s), 1.83-1.49 (8H, m), 1.15 (3H, t).

Compound of the present invention 17: ¹H-NMR (CDCl₃) δ: 7.26 (1H, s), 7.21 (1H, s), 6.58 (1H, s), 6.47 (1H, s), 4.91 (1H, s), 4.27 (2H, s), 4.24 (2H, q), 4.09 (1H, d), 3.90 (1H, d), 2.88-2.60 (2H, m), 2.34 (3H, s), 2.31 (3H, s), 1.86-1.43 (8H, m), 1.29 (3H, t), 1.14 (3H, t).

Compound of the present invention 18: ¹H-NMR (CDCl₃) δ: 7.26 (1H, s), 7.21 (1H, s), 6.54 (1H, s), 6.48 (1H, s), 5.03 (1H, q), 4.27 (2H, s), 3.86 (1H, d), 3.59 (1H, d), 3.21-3.07 (1H, m), 2.34 (3H, s), 2.32 (3H, s), 1.84-1.51 (6H, m), 1.50-1.29 (2H, m), 1.44 (3H, d), 1.15 (3H, d), 1.07 (3H, d).

Compound of the present invention 19: ¹H-NMR (CDCl₃) δ: 7.26 (1H, s), 7.21 (1H, s), 6.54 (1H, s), 6.48 (1H, s), 4.66 (1H, q), 4.27 (2H, s), 3.94 (1H, d), 3.64 (1H, d), 2.77-2.65 (1H, m), 2.56-2.43 (1H, m), 2.34 (3H, s), 2.32 (3H, s), 1.83-1.48 (8H, m), 1.46 (3H, d), 1.15 (3H, t).

Compound of the present invention 20: ¹H-NMR (CDCl₃) δ: 7.51-7.44 (1H, m), 7.17 (1H, s), 6.59 (1H, s), 6.53 (1H, s), 4.29 (2H, s), 3.37 (2H, d), 3.29-3.18 (1H, m), 2.49 (3H, s), 2.46 (3H, s), 2.21 (3H, s), 1.88-1.79 (2H, m), 1.72-1.56 (4H, m), 1.48-1.38 (2H, m), 1.04 (6H, d).

Compound of the present invention 21: ¹H-NMR (CDCl₃) δ: 7.17 (1H, s), 6.60 (1H, s), 6.55 (1H, s), 4.77 (1H, s), 4.57 (1H, s), 4.29 (2H, s), 3.73 (1H, s), 3.49 (1H, s), 3.21-3.09 (1H, m), 2.49 (3H, s), 2.46 (3H, s), 1.85-1.69 (4H, m), 1.68-1.55 (4H, m), 1.08 (3H, d), 1.06 (3H, d).

Compound of the present invention 22: ¹H-NMR (CDCl₃) δ: 7.26 (1H, s), 7.21 (1H, s), 6.57 (1H, s), 6.48 (1H, s), 4.77 (1H, s), 4.56 (1H, s), 4.27 (2H, s), 3.72 (1H, s), 3.48 (1H, s), 3.22-3.10 (1H, m), 2.34 (3H, s), 2.31 (3H, s), 1.85-1.55 (8H, m), 1.08 (3H, d), 1.05 (3H, d).

Compound of the present invention 23: ¹H-NMR (CDCl₃) δ: 7.26 (1H, s), 7.21 (1H, s), 6.53 (1H, s), 6.48 (1H, s), 4.27 (1H, d), 4.27 (2H, s), 3.82 (1H, d), 3.68 (1H, d), 3.24-3.11 (1H, m), 2.34 (3H, s), 2.32 (3H, s), 1.92-1.37 (8H, m), 1.17-0.85 (8H, m), 0.58-0.22 (3H, m).

Compound of the present invention 24: ¹H-NMR (CDCl₃) δ: 7.17 (1H, s), 6.56 (1H, s), 6.55 (1H, s), 4.62 (1H, d), 4.28 (2H, s), 3.83 (1H, d), 3.69 (1H, d), 3.25-3.10 (1H, m), 2.49 (3H, s), 2.46 (3H, s), 1.97-0.85 (16H, m), 0.58-0.22 (3H, m).

Compound of the present invention 25: ¹H-NMR (CDCl₃) δ: 7.26 (1H, s), 7.21 (1H, s), 6.52 (1H, s), 6.48 (1H, s), 4.31 (1H, d), 4.27 (2H, s), 3.91 (1H, d), 3.65 (1H, d), 2.89-2.78 (1H, m), 2.58-2.46 (1H, m), 2.34 (3H, s), 2.32 (3H, s), 1.86-1.60 (5H, m), 1.58-1.41 (2H, m), 1.35-1.24 (1H, m), 1.16 (3H, t), 1.08-0.85 (2H, m), 0.60-0.09 (3H, m).

Compound of the present invention 26: ¹H-NMR (CDCl₃) δ: 7.17 (1H, s), 6.55 (2H, s), 4.31 (1H, d), 4.28 (2H, s), 3.92 (1H, d), 3.66 (1H, d), 2.90-2.78 (1H, m), 2.58-2.50 (1H, m), 2.49 (3H, s), 2.46 (3H, s), 1.88-1.60 (5H, m), 1.58-1.43 (2H, m), 1.37-1.23 (1H, m), 1.16 (3H, t), 1.09-0.85 (2H, m), 0.63-0.21 (3H, m).

Compound of the present invention 27: ¹H-NMR (CDCl₃) δ: 7.17 (1H, s), 6.60 (1H, s), 6.55 (1H, s), 4.62 (1H, s), 4.41 (1H, s), 4.29 (2H, s), 3.82 (1H, s), 3.57 (1H, s), 2.54 (2H, q), 2.49 (3H, s), 2.46 (3H, s), 1.84-1.50 (8H, m), 1.15 (3H, t).

Compound of the present invention 28: ¹H-NMR (CDCl₃) δ: 7.26 (1H, s), 7.21 (1H, s), 6.54 (1H, s), 6.48 (1H, s), 5.04 (1H, dd), 4.27 (2H, s), 3.81 (1H, d), 3.55 (1H, d), 3.25-3.05 (1H, m), 2.34 (3H, s), 2.31 (3H, s), 2.04-1.92 (1H, m), 1.83-1.42 (9H, m), 1.11 (3H, d), 1.03 (3H, d), 0.93 (3H, t).

Compound of the present invention 29: ¹H-NMR (CDCl₃) δ: 7.17 (1H, s), 6.55 (1H, s), 6.52 (1H, s), 4.72 (1H, d), 4.29 (2H, s), 3.97 (1H, d), 3.43 (1H, d), 2.95-2.74 (1H, m), 2.66-2.51 (1H, m), 2.49 (3H, s), 2.46 (3H, s), 2.14-2.02 (1H, m), 1.94-1.36 (6H, m), 1.21-0.77 (11H, m).

Compound of the present invention 30: ¹H-NMR (CDCl₃) δ: 7.25 (1H, s), 7.20 (1H, s), 6.56 (1H, s), 6.47 (1H, s), 5.92-5.57 (1H, m), 5.47-5.16 (2H, m), 4.26 (2H, s), 3.89 (1H, d), 3.60 (1H, d), 3.21-3.12 (1H, m), 2.34 (3H, s), 2.31 (3H, s), 1.85-1.31 (11H, m), 1.14-1.00 (6H, m).

Compound of the present invention 31: ¹H-NMR (CDCl₃) δ: 7.26 (1H, s), 7.21 (1H, s), 6.50 (1H, s), 6.49 (1H, s), 4.71 (1H, d), 4.27 (2H, s), 3.96 (1H, d), 3.42 (1H, d), 2.8-2.73 (1H, m), 2.57-2.46 (1H, m), 2.34 (3H, s), 2.32 (3H, s), 2.13-2.02 (1H, m), 1.83-1.40 (8H, m), 1.11 (3H, t), 1.02 (3H, d), 0.99 (3H, t).

Compound of the present invention 32: ¹H-NMR (CDCl₃) δ: 7.26 (1H, s), 7.21 (1H, s), 6.54 (1H, s), 6.48 (1H, s), 5.06 (1H, dd), 4.27 (2H, s), 3.79 (1H, d), 3.56 (1H, d), 3.27-3.09 (1H, m), 2.34 (3H, s), 2.32 (3H, s), 1.96-1.19 (12H, m), 1.14-0.80 (9H, m).

Compound of the present invention 33: ¹H-NMR (CDCl₃) δ: 7.26 (1H, s), 7.21 (1H, s), 6.54 (1H, s), 6.48 (1H, s), 5.06 (1H, dd), 4.27 (2H, s), 3.78 (1H, d), 3.57 (1H, d), 3.27-3.09 (1H, m), 2.34 (3H, s), 2.32 (3H, s), 1.98-1.19 (14H, m), 1.13-0.80 (9H, m).

Compound of the present invention 34: ¹H-NMR (CDCl₃) δ: 7.52 (1H, s), 7.44 (1H, s), 7.27 (1H, t), 7.21 (1H, t), 6.60 (1H, s), 6.58 (1H, s), 5.03 (1H, q), 4.31 (2H, s), 3.87 (1H, d), 3.60 (1H, d), 3.21-3.08 (1H, m), 1.83-1.37 (11H, m), 1.15 (3H, d), 1.07 (3H, d).

Compound of the present invention 35: ¹H-NMR (CDCl₃) δ: 7.31 (1H, d), 7.30 (1H, s), 7.07 (1H, d), 6.57 (1H, s), 6.52 (1H, s), 5.03 (1H, q), 4.31 (2H, s), 3.87 (1H, d), 3.60 (1H, d), 3.21-3.08 (1H, m), 2.42 (3H, s), 1.83-1.37 (11H, m), 1.15 (3H, d), 1.07 (3H, d).

Compound of the present invention 36: ¹H-NMR (CDCl₃) δ: 7.34 (1H, d), 7.32 (1H, s), 7.10 (1H, d), 6.56 (1H, s), 6.54 (1H, s), 5.03 (1H, q), 4.31 (2H, s), 3.86 (1H, d), 3.60 (1H, d), 3.21-3.08 (1H, m), 2.72 (2H, q), 1.83-1.40 (11H, m), 1.26 (3H, t), 1.15 (3H, d), 1.07 (3H, d).

Compound of the present invention 37: ¹H-NMR (CDCl₃) δ: 7.36 (1H, s), 7.35 (1H, d), 7.14 (1H, d), 6.56 (1H, s), 6.55 (1H, s), 5.03 (1H, q), 4.29 (2H, s), 3.86 (1H, d), 3.59 (1H, d), 3.21-3.08 (1H, m), 3.05-2.93 (1H, m), 1.82-1.37 (11H, m), 1.28 (6H, d), 1.15 (3H, d), 1.07 (3H, d).

Compound of the present invention 38: ¹H-NMR (CDCl₃) δ: 7.43 (1H, dd), 7.16 (1H, dd), 6.99 (1H, td), 6.57 (2H, s), 5.04 (1H, q), 4.29 (2H, s), 3.87 (1H, d), 3.60 (1H, d), 3.21-3.08 (1H, m), 1.84-1.30 (11H, m), 1.15 (3H, d), 1.07 (3H, d).

Compound of the present invention 39: ¹H-NMR (CDCl₃) δ: 7.49 (1H, d), 7.35 (1H, d), 7.23 (1H, dd), 6.59 (1H, s), 6.55 (1H, s), 5.04 (1H, q), 4.30 (2H, s), 3.88 (1H, d), 3.60 (1H, d), 3.21-3.08 (1H, m), 1.83-1.28 (11H, m), 1.15 (3H, d), 1.07 (3H, d).

Compound of the present invention 40: ¹H-NMR (CDCl₃) δ: 7.37 (1H, d), 6.98 (1H, d), 6.85 (1H, dd), 6.56 (1H, s), 6.52 (1H, s), 5.03 (1H, q), 4.27 (2H, s), 3.87 (1H, d), 3.84 (3H, s), 3.60 (1H, d), 3.20-3.09 (1H, m), 1.82-1.29 (11H, m), 1.15 (3H, d), 1.07 (3H, d).

Compound of the present invention 41: ¹H-NMR (CDCl₃) δ: 7.23 (1H, s), 6.93 (1H, s), 6.55 (1H, s), 6.47 (1H, s), 5.03 (1H, q), 4.26 (2H, s), 3.87 (1H, d), 3.85 (3H, s), 3.59 (1H, d), 3.21-3.07 (1H, m), 2.27 (3H, s), 1.82-1.24 (11H, m), 1.14 (3H, d), 1.07 (3H, d).

Compound of the present invention 42: ¹H-NMR (CDCl₃) δ: 7.38 (1H, d), 7.24 (1H, s), 7.04 (1H, d), 6.56 (1H, s), 6.54 (1H, s), 5.03 (1H, q), 4.28 (2H, s), 3.87 (1H, d), 3.60 (1H, d), 3.21-3.09 (1H, m), 2.46 (3H, s), 1.82-1.30 (11H, m), 1.15 (3H, d), 1.07 (3H, d).

Compound of the present invention 43: ¹H-NMR (CDCl₃) δ: 7.53 (1H, s), 7.47 (1H, s), 7.05 (1H, s), 6.52 (1H, s), 5.03 (1H, q), 4.36 (2H, s), 3.86 (1H, d), 3.59 (1H, d), 3.21-3.08 (1H, m), 2.35 (3H, s), 2.34 (3H, s), 1.82-1.24 (11H, m), 1.14 (3H, d), 1.06 (3H, d).

Compound of the present invention 44: ¹H-NMR (CDCl₃) δ: 7.51 (1H, d), 7.23 (1H, s), 7.12 (1H, d), 6.53 (1H, s), 5.03 (1H, q), 4.39 (2H, s), 3.86 (1H, d), 3.59 (1H, d), 3.22-3.08 (1H, m), 2.47 (3H, s), 2.38 (3H, s), 1.85-1.25 (11H, m), 1.14 (3H, d), 1.06 (3H, d).

Compound of the present invention 45: ¹H-NMR (CDCl₃) δ: 7.10 (1H, s), 6.55 (1H, s), 6.54 (1H, s), 5.03 (1H, q), 4.27 (2H, s), 3.86 (1H, d), 3.59 (1H, d), 3.21-3.07 (1H, m), 2.39 (3H, s), 2.37 (3H, s), 2.23 (3H, s), 1.84-1.24 (11H, m), 1.14 (3H, d), 1.06 (3H, d).

Compound of the present invention 46: ¹H-NMR (CDCl₃) δ: 7.07 (1H, s), 6.85 (1H, s), 6.56 (1H, s), 6.55 (1H, s), 5.03 (1H, q), 4.28 (2H, s), 3.87 (1H, d), 3.60 (1H, d), 3.23-3.08 (1H, m), 2.43 (3H, s), 2.41 (3H, s), 1.85-1.28 (11H, m), 1.15 (3H, d), 1.07 (3H, d).

Compound of the present invention 47: ¹H-NMR (CDCl₃) δ: 7.49 (1H, s), 7.30 (1H, s), 6.57 (1H, s), 6.50 (1H, s), 5.03 (1H, q), 4.28 (2H, s), 3.87 (1H, d), 3.60 (1H, d), 3.22-3.08 (1H, m), 2.45 (3H, s), 1.84-1.28 (11H, m), 1.14 (3H, d), 1.07 (3H, d).

Compound of the present invention 48: ¹H-NMR (CDCl₃) δ: 7.36 (1H, dd), 7.17 (1H, dd), 6.98 (1H, td), 6.58 (1H, s), 6.57 (1H, s), 5.03 (1H, q), 4.30 (2H, s), 3.88 (1H, d), 3.60 (1H, d), 3.21-3.08 (1H, m), 1.87-1.29 (11H, m), 1.15 (3H, d), 1.07 (3H, d).

Compound of the present invention 49: ¹H-NMR (CDCl₃) δ: 7.48 (1H, s), 7.38 (1H, d), 7.24 (1H, m), 7.03 (1H, d), 6.58 (1H, t), 6.52 (1H, q), 4.28 (2H, s), 3.49 (2H, d), 2.45 (3H, s), 1.74-1.25 (8H, m), 1.06 (9H, s).

Compound of the present invention 50: ¹H-NMR (CDCl₃) δ: 7.49 (1H, s), 7.48-7.46 (2H, m), 7.38 (1H, s), 7.33 (1H, s), 7.26-7.23 (1H, m), 6.96-6.92 (2H, m), 6.81 (1H, s), 6.72-6.68 (1H, m), 5.23 (2H, s), 3.50 (2H, d), 1.73-1.26 (8H, m), 1.07 (9H, s).

Compound of the present invention 51: ¹H-NMR (CDCl₃) δ: 7.48 (1H, s), 7.28-7.22 (2H, m), 7.21 (1H, s), 6.57 (1H, t), 6.46 (1H, s), 4.26 (2H, s), 3.48 (2H, d), 2.34 (3H, s), 2.31 (3H, s), 1.75-1.55 (8H, m), 1.06 (9H, s).

Compound of the present invention 52: ¹H-NMR (CDCl₃) δ: 7.17 (1H, s), 6.55 (1H, s), 6.52 (1H, s), 5.04 (1H, d), 4.28 (2H, s), 3.78 (1H, d), 3.58 (1H, d), 3.20-3.09 (1H, m), 2.66-2.52 (1H, m), 2.49 (3H, s), 2.46 (3H, s), 1.99-0.68 (20H, m).

Compound of the present invention 53: ¹H-NMR (CDCl₃) δ: 7.17 (1H, s), 6.59 (1H, s), 6.55 (1H, s), 5.19 (1H, dd), 4.29 (2H, s), 3.87 (1H, d), 3.79-3.06 (7H, m), 2.49 (3H, s), 2.46 (3H, s), 1.84-1.41 (8H, m), 1.16 (3H, d), 1.02 (3H, d).

Compound of the present invention 54: ¹H-NMR (CDCl₃) δ: 7.33-7.09 (6H, m), 6.57 (1H, s), 6.56 (1H, s), 5.23 (1H, dd), 4.29 (2H, s), 3.63 (1H, d), 3.33-2.88 (4H, m), 2.50 (3H, s), 2.46 (3H, s), 1.95-1.40 (8H, m), 0.97 (3H, d), 0.78 (3H, d).

Compound of the present invention 55: ¹H-NMR (CDCl₃) δ: 8.16 (1H, d), 7.93 (1H, d), 7.53 (1H, t), 7.51 (1H, d), 6.96 (1H, dd), 6.89 (1H, d), 5.39 (2H, s), 4.64 (1H, s), 4.41 (1H, s), 3.86 (1H, s), 3.60 (1H, s), 2.36 (3H, s), 1.86-1.47 (8H, m).

Compound of the present invention 56: ¹H-NMR (CDCl₃) δ: 6.36 (1H, t), 4.44 (1H, d), 4.02 (1H, d), 3.40 (1H, d), 2.78 (2H, t), 2.31 (3H, s), 2.27-2.19 (1H, m), 1.74-1.38 (12H, m), 1.02 (6H, d), 0.95 (3H, t).

Compound of the present invention 57: ¹H-NMR (CDCl₃) δ: 7.17-7.13 (4H, m), 6.29 (1H, s), 4.70 (1H, d), 4.06 (2H, s), 3.96 (1H, d), 3.41 (1H, d), 2.82-2.74 (1H, m), 2.56-2.49 (1H, m), 2.34 (3H, s), 2.11-2.03 (1H, m), 1.78-1.43 (8H, m), 1.10 (3H, t), 0.99 (6H, d).

Compound of the present invention 58: ¹H-NMR (CDCl₃) δ: 7.49 (1H, s), 7.28 (1H, s), 7.17 (1H, s), 6.59 (1H, t), 6.53 (1H, d), 4.28 (2H, s), 3.49 (2H, d), 2.49 (3H, s), 2.46 (3H, s), 1.69-1.61 (8H, m), 1.06 (9H, s).

Compound of the present invention 59: ¹H-NMR (CDCl₃) δ: 7.17 (1H, s), 6.60 (1H, d), 6.55 (1H, t), 4.82 (1H, s), 4.61 (1H, s), 4.29 (2H, s), 3.75 (1H, s), 3.51 (1H, s), 2.49 (3H, s), 2.46 (3H, s), 1.73-1.63 (8H, m).

Compound of the present invention 60: ¹H-NMR (CDCl₃) δ: 7.17 (1H, s), 6.56 (1H, d), 6.53 (1H, t), 4.43 (1H, d), 4.29 (2H, s), 4.02 (1H, d), 3.41 (1H, d), 2.49 (3H, s), 2.46 (3H, s), 2.31 (3H, s), 2.25-2.21 (1H, m), 1.76-1.26 (8H, m), 1.02 (3H, s), 1.00 (3H, s).

Compound of the present invention 61: ¹H-NMR (CDCl₃) δ: 7.17 (1H, s), 6.60 (1H, d), 6.55 (1H, dd), 4.61 (1H, s), 4.39 (1H, s), 4.29 (2H, s), 3.83 (1H, s), 3.58 (1H, s), 2.49 (3H, s), 2.46 (3H, s), 2.33 (3H, d), 1.79-1.53 (8H, m)).

Compound of the present invention 62: ¹H-NMR (CDCl₃) δ: 8.15 (1H, d), 7.92 (1H, d), 7.52 (1H, t), 7.50 (1H, d), 6.96 (1H, d), 6.83 (1H, s), 5.38 (2H, s), 4.45 (1H, d), 4.05 (1H, d), 3.44 (1H, d), 2.32 (3H, s), 2.28-2.21 (1H, m), 1.78-1.26 (8H, m), 1.03 (3H, s), 1.02 (3H, s).

Compound of the present invention 63: ¹H-NMR (CDCl₃) δ: 7.92 (1H, d), 7.73 (1H, d), 7.67 (2H, m), 7.36 (2H, m), 6.47 (1H, m), 6.36 (1H, s), 4.42 (1H, d), 4.15 (2H, s), 4.01 (1H, d), 3.40 (1H, d), 2.30 (3H, s), 2.26-2.18 (1H, m), 1.75-1.24 (8H, m), 1.01 (3H, s), 0.99 (3H, s).

Compound of the present invention 64: ¹H-NMR (CDCl₃) δ: 8.45 (1H, d), 6.80 (1H, d), 5.06 (1H, q), 3.88 (1H, d), 3.63 (1H, d), 3.23-3.09 (1H, m), 1.86-1.53 (6H, m), 1.51-1.41 (5H, m), 1.16 (3H, d), 1.08 (3H, d).

Compound of the present invention 65: ¹H-NMR (CDCl₃) δ: 7.92 (1H, d), 7.73 (1H, d), 7.67 (2H, d), 7.36 (2H, d), 6.47 (1H, t), 6.40 (1H, s), 5.03 (1H, q), 4.15 (2H, s), 3.86 (1H, d), 3.59 (1H, d), 3.22-3.07 (1H, m), 1.88-1.35 (11H, m), 1.14 (3H, d), 1.06 (3H, d).

Compound of the present invention 66: ¹H-NMR (CDCl₃) δ: 8.55 (1H, d), 8.34 (1H, d), 7.97 (1H, d), 7.77-7.67 (2H, m), 6.47 (1H, t), 6.44 (1H, s), 5.03 (1H, q), 4.15 (2H, s), 3.86 (1H, d), 3.59 (1H, d), 3.22-3.07 (1H, m), 1.86-1.28 (11H, m), 1.14 (3H, d), 1.06 (3H, d).

Compound of the present invention 67: ¹H-NMR (CDCl₃) δ: 8.16 (1H, d), 7.92 (1H, d), 7.55-7.49 (2H, m), 6.96 (1H, d), 6.86 (1H, s), 5.38 (2H, s), 5.06 (1H, q), 3.90 (1H, d), 3.63 (1H, d), 3.19-3.12 (1H, m), 1.76-1.56 (8H, m), 1.46 (3H, d), 1.16 (3H, d), 1.08 (3H, d).

Compound of the present invention 68: ¹H-NMR (CDCl₃) δ: 7.58-7.54 (5H, m), 7.45-7.41 (2H, m), 7.35-7.33 (2H, m), 6.41 (1H, s), 5.03 (1H, q), 4.15 (2H, s), 3.88 (1H, d), 3.60 (1H, d), 3.18-3.09 (1H, m), 1.78-1.45 (8H, m), 1.44 (3H, d), 1.14 (3H, d), 1.06 (3H, d).

Compound of the present invention 69: ¹H-NMR (CDCl₃) δ: 7.30 (2H, d), 7.19 (2H, d), 6.39 (1H, s), 5.03 (1H, q), 4.12 (2H, s), 3.87 (1H, d), 3.59 (1H, d), 3.17-3.10 (1H, m), 1.79-1.56 (8H, m), 1.44 (3H, d), 1.14 (3H, d), 1.06 (3H, d).

Compound of the present invention 70: ¹H-NMR (CDCl₃) δ: 7.15 (4H, m), 6.34 (1H, s), 5.02 (1H, q), 4.06 (2H, s), 3.86 (1H, d), 3.58 (1H, d), 3.16-3.09 (1H, m), 2.34 (3H, s), 1.78-1.54 (8H, m), 1.43 (3H, d), 1.14 (3H, d), 1.06 (3H, d).

Compound of the present invention 71: ¹H-NMR (CDCl₃) δ: 7.26 (1H, s), 7.21 (1H, s), 6.55 (1H, s), 6.48 (1H, s), 5.14 (1H, t), 4.26 (2H, s), 3.75 (1H, d), 3.67 (1H, d), 3.28-3.13 (1H, m), 2.34 (3H, s), 2.32 (3H, s), 2.28-2.17 (1H, m), 1.97-1.21 (10H, m), 1.15-0.80 (12H, m).

Compound of the present invention 72: ¹H-NMR (CDCl₃) δ: 7.18 (2H, d), 6.97 (2H, d), 6.78 (1H, s), 5.18 (2H, s), 5.05 (1H, q), 3.87 (1H, d), 3.61 (1H, d), 3.18-3.11 (1H, m), 1.83-1.56 (8H, m), 1.46 (3H, d), 1.15 (3H, d), 1.08 (3H, d).

Compound of the present invention 73: ¹H-NMR (CDCl₃) δ: 7.26 (1H, s), 7.21 (1H, s), 6.52 (1H, s), 6.49 (1H, s), 5.07 (1H, d), 4.27 (2H, s), 4.05 (1H, d), 3.58 (1H, dd), 3.48 (1H, dd), 3.43 (1H, d), 2.34 (3H, s), 2.32 (3H, s), 2.28-2.15 (2H, m), 1.95-1.40 (7H, m), 1.32-1.25 (1H, m), 1.02 (3H, d), 1.00 (3H, d).

Compound of the present invention 74: ¹H-NMR (CDCl₃) δ: 7.32-7.28 (2H, m), 7.23-7.18 (3H, m), 6.42 (1H, s), 5.04 (1H, q), 3.87 (1H, d), 3.60 (1H, d), 3.17-3.10 (1H, m), 2.80 (2H, t), 2.71 (2H, t), 2.10-2.02 (2H, m), 1.82-1.55 (8H, m), 1.45 (3H, d), 1.15 (3H, d), 1.08 (3H, d).

Compound of the present invention 75: ¹H-NMR (CDCl₃) δ: 7.15 (1H, s), 7.11-7.06 (2H, m), 6.52-6.45 (2H, m), 6.25-6.18 (1H, m), 5.04 (1H, q), 3.86 (1H, d), 3.67 (2H, d), 3.60 (1H, d), 3.17-3.10 (1H, m), 2.25 (6H, d), 1.76-1.55 (8H, m), 1.45 (3H, d), 1.15 (3H, d), 1.07 (3H, d).

Compound of the present invention 76: ¹H-NMR (CDCl₃) δ: 6.41 (1H, s), 5.05 (1H, q), 3.89 (1H, d), 3.61 (1H, d), 3.17-3.10 (1H, m), 2.67 (2H, d), 2.10-2.03 (1H, m), 1.78-1.56 (8H, m), 1.45 (3H, d), 1.15 (3H, d), 1.08 (3H, d), 0.99 (3H, s), 0.97 (3H, s).

Compound of the present invention 77: ¹H-NMR (CDCl₃) δ: 7.37-7.26 (5H, m), 6.36 (1H, t), 5.02 (1H, q), 4.11 (2H, s), 3.87 (1H, d), 3.59 (1H, d), 3.16-3.10 (1H, m), 1.78-1.54 (8H, m), 1.42 (3H, d), 1.14 (3H, d), 1.06 (3H, d).

Compound of the present invention 78: ¹H-NMR (CDCl₃) δ: 8.70-8.68 (1H, m), 7.98-7.96 (2H, m), 7.78-7.70 (2H, m), 7.38 (2H, d), 7.24-7.22 (1H, m), 6.40 (1H, s), 5.03 (1H, q), 4.17 (2H, s), 3.86 (1H, d), 3.59 (1H, d), 3.18-3.10 (1H, m), 1.81-1.45 (8H, m), 1.44 (3H, d), 1.14 (3H, d), 1.06 (3H, d).

Compound of the present invention 79: ¹H-NMR (CDCl₃) δ: 7.55 (1H, s), 7.51 (1H, d), 7.30 (1H, d), 6.99 (1H, s), 6.88-6.61 (1H, t), 6.37 (1H, s), 5.02 (1H, q), 4.20 (2H, s), 3.86 (1H, d), 3.59 (1H, d), 3.16-3.09 (1H, m), 1.78-1.54 (8H, m), 1.43 (3H, d), 1.14 (3H, d), 1.06 (3H, d).

Compound of the present invention 80: ¹H-NMR (CDCl₃) δ: 7.26 (1H, s), 7.21 (1H, s), 6.60 (1H, s), 6.47 (1H, s), 5.24 (1H, s), 4.27 (2H, s), 4.22 (2H, q), 3.96 (1H, d), 3.87 (1H, d), 3.37-3.20 (1H, m), 2.34 (3H, s), 2.31 (3H, s), 1.92-1.48 (8H, m), 1.28 (3H, t), 1.15 (3H, d), 1.09 (3H, d).

Compound of the present invention 81: ¹H-NMR (CDCl₃) δ: 7.17 (1H, s), 6.62 (1H, s), 6.54 (1H, s), 5.24 (1H, s), 4.29 (2H, s), 4.22 (2H, q), 3.97 (1H, d), 3.88 (1H, d), 3.37-3.21 (1H, m), 2.49 (3H, s), 2.46 (3H, s), 1.92-1.47 (8H, m), 1.29 (3H, t), 1.15 (3H, d), 1.09 (3H, d).

Compound of the present invention 82: ¹H-NMR (CDCl₃) δ: 7.48-7.46 (2H, m), 7.38 (1H, s), 7.26-7.23 (1H, m), 6.96-6.93 (2H, m), 6.81 (1H, s), 6.73-6.69 (1H, m), 5.24 (2H, s), 5.05 (1H, q), 3.88 (1H, d), 3.62 (1H, d), 3.18-3.11 (1H, m), 1.83-1.58 (8H, m), 1.46 (3H, d), 1.15 (3H, d), 1.08 (3H, d).

Compound of the present invention 83: ¹H-NMR (CDCl₃) δ: 6.88-6.87 (2H, m), 6.78 (1H, s), 6.72-6.68 (1H, m), 5.17 (2H, s), 5.05 (1H, q), 3.87 (1H, d), 3.61 (1H, d), 3.19-3.12 (1H, m), 3.12 (1H, s), 1.82-1.56 (8H, m), 1.46 (3H, d), 1.15 (3H, d), 1.08 (3H, d).

Compound of the present invention 84: ¹H-NMR (CDCl₃) δ: 7.39-7.17 (7H, m), 6.52 (1H, s), 6.50 (1H, s), 4.79 (1H, d), 4.28 (2H, s), 4.07 (1H, d), 4.00 (1H, d), 3.61 (1H, d), 3.50 (1H, d), 2.35 (3H, s), 2.32 (3H, s), 1.81-1.15 (9H, m), 0.90 (3H, d), 0.86 (3H, d).

Compound of the present invention 85: ¹H-NMR (CDCl₃) δ: 7.46-7.42 (2H, m), 7.19-7.13 (1H, m), 6.70 (1H, s), 5.05 (1H, q), 4.66 (2H, s), 4.56 (2H, s), 3.87 (1H, d), 3.61 (1H, d), 3.18-3.11 (1H, m), 1.77-1.56 (8H, m), 1.46 (3H, d), 1.16 (3H, d), 1.08 (3H, d).

Compound of the present invention 86: ¹H-NMR (CDCl₃) δ: 7.41-7.18 (7H, m), 6.54 (1H, s), 6.49 (1H, s), 4.64 (1H, q), 4.27 (2H, s), 4.02 (1H, d), 3.93 (1H, d), 3.69 (1H, d), 3.53 (1H, d), 2.34 (3H, s), 2.32 (3H, s), 1.82-1.36 (8H, m), 1.17 (3H, d).

Compound of the present invention 87: ¹H-NMR (CDCl₃) δ: 7.37-7.18 (7H, m), 6.55 (1H, s), 6.47 (1H, s), 4.49 (1H, s), 4.27 (2H, s), 4.22 (1H, s), 3.88 (1H, s), 3.65 (2H, s), 3.63 (1H, s), 2.34 (3H, s), 2.31 (3H, s), 1.93-1.54 (8H, m).

Compound of the present invention 88: ¹H-NMR (CDCl₃) δ: 7.37-7.16 (7H, m), 6.58 (1H, s), 6.47 (1H, s), 4.80 (1H, s), 4.27 (1H, d), 4.27 (2H, s), 3.97 (2H, m), 3.91-3.80 (1H, m), 3.73 (1H, d), 3.64-3.47 (1H, d), 2.34 (3H, s), 2.31 (3H, s), 1.88-1.50 (8H, m), 0.97 (3H, t).

Compound of the present invention 89: ¹H-NMR (CDCl₃) δ: 7.37 (1H, d), 7.25 (1H, s), 7.19 (1H, s), 6.53 (1H, s), 6.46 (1H, s), 6.32 (1H, dd), 6.08-5.96 (1H, m), 5.52 (1H, s), 4.25 (2H, s), 4.14 (1H, d), 3.86 (1H, d), 2.76-58 (1H, m), 2.57-2.43 (1H, m), 2.34 (3H, s), 2.31 (3H, s), 1.85-1.36 (8H, m), 0.86 (3H, t).

Compound of the present invention 90: ¹H-NMR (CDCl₃) δ: 7.51 (2H, d), 7.24 (1H, s), 7.18 (1H, s), 6.84 (2H, d), 6.49 (1H, s), 6.44 (1H, s), 5.24 (1H, s), 4.23 (2H, s), 4.17 (1H, d), 3.79 (1H, d), 3.78 (3H, s), 2.78-2.61 (1H, m), 2.53-2.36 (1H, m), 2.33 (3H, s), 2.31 (3H, s), 1.90-1.57 (8H, m), 0.69 (3H, t).

Compound of the present invention 91: ¹H-NMR (CDCl₃) δ: 7.58 (2H, d), 7.33-6.98 (5H, m), 6.49 (1H, s), 6.44 (1H, s), 5.29 (1H, s), 4.23 (2H, s), 4.19 (1H, d), 3.81 (1H, d), 2.80-2.62 (1H, m), 2.56-2.38 (1H, m), 2.33 (3H, s), 2.30 (3H, s), 1.89-1.22 (8H, m), 0.67 (3H, t).

Compound of the present invention 92: ¹H-NMR (CDCl₃) δ: 7.58 (1H, s), 7.32 (1H, t), 7.25 (1H, s), 7.20 (1H, s), 6.54 (1H, d), 6.52 (1H, s), 6.46 (1H, s), 5.45 (1H, s), 4.25 (2H, s), 4.12 (1H, d), 3.67 (1H, d), 2.80-2.63 (1H, m), 2.57-2.39 (1H, m), 2.34 (3H, s), 2.31 (3H, s), 1.87-1.47 (8H, m), 0.92 (3H, t).

Compound of the present invention 93: ¹H-NMR (CDCl₃) δ: 8.33 (1H, d), 7.83 (1H, d), 7.69 (1H, dd), 6.43 (1H, s), 5.99 (1H, s), 5.03 (1H, q), 4.13 (2H, s), 3.86 (1H, d), 3.59 (1H, d), 3.19-3.08 (1H, m), 2.62 (3H, s), 2.29 (3H, s), 1.89-1.50 (8H, m), 1.44 (3H, s), 1.14 (3H, d), 1.07 (3H, d).

Compound of the present invention 94: ¹H-NMR (CDCl₃) δ: 7.71 (2H, d), 7.56 (2H, d), 7.24 (1H, s), 7.18 (1H, s), 6.48 (1H, s), 6.44 (1H, s), 5.34 (1H, s), 4.24 (2H, s), 4.22 (1H, d), 3.80 (1H, d), 2.82-2.69 (1H, m), 2.55-2.41 (1H, m), 2.33 (3H, s), 2.31 (3H, s), 1.90-1.57 (7H, m), 1.44-1.36 (1H, m), 0.67 (3H, t).

Compound of the present invention 95: ¹H-NMR (CDCl₃) δ: 7.31 (1H, dd), 7.24 (1H, s), 7.21 (1H, dd), 7.19 (1H, s), 6.92 (1H, dd), 6.52 (1H, s), 6.45 (1H, s), 5.75 (1H, s), 4.24 (2H, s), 4.22 (1H, d), 3.70 (1H, d), 2.86-2.68 (1H, m), 2.65-2.46 (1H, m), 2.34 (3H, s), 2.31 (3H, s), 1.91-1.50 (7H, m), 1.38-1.24 (1H, m), 0.92 (3H, t).

Compound of the present invention 96: ¹H-NMR (CDCl₃) δ: 7.35 (1H, dd), 7.25 (1H, s), 7.20 (1H, s), 6.55 (1H, s), 6.46 (1H, s), 6.37 (1H, t), 6.30 (1H, t), 6.00 (1H, s), 4.25 (2H, s), 4.10 (1H, d), 3.76 (1H, d), 3.28-3.17 (1H, m), 2.34 (3H, s), 2.31 (3H, s), 1.81-1.44 (8H, m), 1.12 (3H, d), 0.88 (3H, d).

Compound of the present invention 97: ¹H-NMR (CDCl₃) δ: 8.29 (1H, d), 7.80 (1H, dd), 7.62 (1H, dd), 7.58 (2H, d), 7.33-7.03 (3H, m), 6.37 (1H, s), 5.98 (1H, s), 5.29 (1H, s), 4.19 (1H, d), 4.09 (2H, s), 3.81 (1H, d), 2.83-2.66 (1H, m), 2.60 (3H, s), 2.56-2.35 (1H, m), 2.28 (3H, s), 1.90-1.49 (7H, m), 1.42-1.34 (1H, m), 0.67 (3H, t).

Compound of the present invention 98: ¹H-NMR (CDCl₃) δ: 7.26 (1H, s), 7.22 (1H, s), 6.48 (2H, s), 4.78 (1H, s), 4.27 (2H, s), 4.05 (1H, d), 3.42 (1H, d), 3.01-2.90 (1H, m), 2.78-2.66 (1H, m), 2.34 (3H, s), 2.32 (3H, s), 1.92-1.21 (8H, m), 1.09 (3H, t), 0.98 (9H, s).

Compound of the present invention 99: ¹H-NMR (CDCl₃) δ: 7.26 (1H, s), 7.22 (1H, s), 6.49 (1H, s), 6.48 (1H, s), 4.56 (1H, s), 4.27 (2H, s), 4.03 (1H, d), 3.39 (1H, d), 2.38 (3H, s), 2.35 (3H, s), 2.32 (3H, s), 1.80-1.35 (8H, m), 0.98 (9H, s).

Compound of the present invention 100: ¹H-NMR (CDCl₃) δ: 7.26 (1H, s), 7.22 (1H, s), 6.49 (1H, s), 6.47 (1H, s), 4.59 (1H, s), 4.27 (2H, s), 3.93 (1H, d), 3.42 (1H, d), 2.35 (3H, s), 2.34 (3H, s), 2.32 (3H, s), 1.13 (3H, s), 0.97 (9H, s), 0.89 (3H, s).

Compound of the present invention 101: ¹H-NMR (CDCl₃) δ: 7.26 (1H, s), 7.21 (1H, s), 6.58 (1H, s), 6.48 (1H, s), 4.60 (1H, s), 4.34-4.08 (5H, m), 3.77 (1H, d), 2.39 (3H, s), 2.34 (3H, s), 2.31 (3H, s), 1.85-1.57 (7H, m), 1.39-1.30 (1H, m), 1.28 (3H, t).

Compound of the present invention 102: ¹H-NMR (CDCl₃) δ: 7.49 (1H, s), 7.29 (1H, s), 6.60 (1H, s), 6.50 (1H, s), 4.61 (1H, s), 4.34-4.06 (5H, m), 3.78 (1H, d), 2.45 (3H, s), 2.40 (3H, s), 1.86-1.54 (7H, m), 1.40-1.32 (1H, m), 1.29 (3H, t).

Compound of the present invention 103: ¹H-NMR (CDCl₃) δ: 7.49 (1H, s), 7.30 (1H, s), 6.51 (2H, s), 4.57 (1H, s), 4.28 (2H, s), 4.05 (1H, d), 3.40 (1H, d), 2.46 (3H, s), 2.39 (3H, s), 1.88-1.35 (8H, m), 0.98 (9H, s).

Compound of the present invention 104: ¹H-NMR (CDCl₃) δ: 7.26 (1H, s), 7.21 (1H, s), 6.51 (1H, s), 6.49 (1H, s), 5.18 (1H, s), 4.27 (2H, s), 4.11 (1H, d), 3.64 (1H, dd), 3.47 (1H, dd), 3.40 (1H, d), 2.34 (3H, s), 2.32 (3H, s), 2.28 (1H, t), 1.94-1.34 (8H, m), 0.99 (9H, s).

Compound of the present invention 105: ¹H-NMR (CDCl₃) δ: 7.26 (1H, s), 7.21 (1H, s), 6.60 (1H, s), 6.48 (1H, s), 5.22 (1H, s), 4.43-4.05 (5H, m), 3.98-3.50 (3H, m), 2.34 (3H, s), 2.32 (3H, s), 2.26 (1H, t), 2.20-1.91 (1H, m), 1.85-1.49 (7H, m), 1.30 (3H, t).

Compound of the present invention 106: ¹H-NMR (CDCl₃) δ: 8.34 (1H, d), 7.84 (1H, d), 7.69 (1H, dd), 6.36 (1H, s), 5.99 (1H, s), 4.56 (1H, s), 4.14 (2H, s), 4.02 (1H, d), 3.39 (1H, d), 2.62 (3H, s), 2.39 (3H, s), 2.29 (3H, s), 1.80-1.35 (8H, m), 0.98 (9H, s).

Compound of the present invention 107: ¹H-NMR (CDCl₃) δ: 7.26 (1H, s), 7.22 (1H, s), 6.51 (1H, s), 6.49 (1H, s), 4.42 (1H, d), 4.27 (2H, s), 4.01 (1H, d), 3.40 (1H, d), 2.35 (3H, s), 2.32 (3H, s), 2.31 (3H, s), 2.28-2.13 (1H, m), 1.80-1.42 (8H, m), 1.00 (6H, d).

Compound of the present invention 108: ¹H-NMR (CDCl₃) δ: 7.26 (1H, s), 7.21 (1H, s), 6.58 (1H, s), 6.48 (1H, s), 4.60 (1H, s), 4.39 (1H, s), 4.27 (2H, s), 3.82 (1H, s), 3.58 (1H, s), 2.34 (6H, s), 2.32 (3H, s), 1.85-1.48 (8H, m).

Then, examples of the Compound of the present invention produced in accordance with any of the production examples described in Examples and the production methods described in the present specification are shown below.

A compound represented by formula (L-1) (hereinafter, referred to as a compound (L-1)),

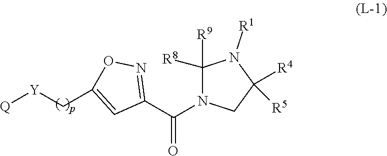

wherein Y represents an oxygen atom, p represents 1, Q represents a phenyl group, and the combination of R¹, R⁴, R⁵, R³, and R⁹ is any one of the combinations shown in [Table L1] to [Table L4](hereinafter, referred to as a compound group SX1).

TABLE L1

| R¹ | R⁴ | R⁵ | R⁸ | R⁹ |
|---|---|---|---|---|
| H | Me | Me | H | H |
| H | Et | Me | H | H |
| H | Et | Et | H | H |
| H | —(CH₂)₄— | | H | H |
| H | Me | Me | Me | H |
| H | Et | Me | Me | H |
| H | Et | Et | Me | H |
| H | —(CH₂)₄— | | Me | H |
| H | Me | Me | Me | Me |
| H | Et | Me | Me | Me |
| H | Et | Et | Me | Me |
| H | —(CH₂)₄— | | Me | Me |
| H | Me | Me | Et | H |
| H | Et | Me | Et | H |
| H | Et | Et | Et | H |
| H | —(CH₂)₄— | | Et | H |
| H | Me | Me | Pr | H |
| H | Et | Me | Pr | H |
| H | Et | Et | Pr | H |
| H | —(CH₂)₄— | | Pr | H |
| H | Me | Me | i-Pr | H |
| H | Et | Me | i-Pr | H |
| H | Et | Et | i-Pr | H |
| H | —(CH₂)₄— | | i-Pr | H |
| H | Me | Me | t-Bu | H |
| H | Et | Me | t-Bu | H |
| H | Et | Et | t-Bu | H |

| | | | |
|---|---|---|---|
| H | —(CH₂)₄— | t-Bu | H |

TABLE L2

| R¹ | R⁴ | R⁵ | R⁸ | R⁹ |
|---|---|---|---|---|
| Me | Me | Me | H | H |
| Me | Et | Me | H | H |
| Me | Et | Et | H | H |
| Me | —(CH₂)₄— | | H | H |
| Me | Me | Me | Me | H |
| Me | Et | Me | Me | H |
| Me | Et | Et | Me | H |
| Me | —(CH₂)₄— | | Me | H |
| Me | Me | Me | Me | Me |
| Me | Et | Me | Me | Me |
| Me | Et | Et | Me | Me |
| Me | —(CH₂)₄— | | Me | Me |
| Me | Me | Me | Et | H |
| Me | Et | Me | Et | H |
| Me | Et | Et | Et | H |
| Me | —(CH₂)₄— | | Et | H |
| Me | Me | Me | Pr | H |
| Me | Et | Me | Pr | H |
| Me | Et | Et | Pr | H |
| Me | —(CH₂)₄— | | Pr | H |
| Me | Me | Me | i-Pr | H |
| Me | Et | Me | i-Pr | H |
| Me | Et | Et | i-Pr | H |
| Me | —(CH₂)₄— | | i-Pr | H |
| Me | Me | Me | t-Bu | H |
| Me | Et | Me | t-Bu | H |
| Me | Et | Et | t-Bu | H |

| | | | |
|---|---|---|---|
| Me | —(CH₂)₄— | t-Bu | H |

TABLE L3

| R¹ | R⁴ | R⁵ | R⁸ | R⁹ |
|---|---|---|---|---|
| Et | Me | Me | H | H |
| Et | Et | Me | H | H |
| Et | Et | Et | H | H |
| Et | —(CH₂)₄— | | H | H |
| Et | Me | Me | Me | H |
| Et | Et | Me | Me | H |
| Et | Et | Et | Me | H |
| Et | —(CH₂)₄— | | Me | H |
| Et | Me | Me | Me | Me |
| Et | Et | Me | Me | Me |
| Et | Et | Et | Me | Me |
| Et | —(CH₂)₄— | | Me | Me |
| Et | Me | Me | Et | H |
| Et | Et | Me | Et | H |
| Et | Et | Et | Et | H |
| Et | —(CH₂)₄— | | Et | H |
| Et | Me | Me | Pr | H |
| Et | Et | Me | Pr | H |
| Et | Et | Et | Pr | H |
| Et | —(CH₂)₄— | | Pr | H |
| Et | Me | Me | i-Pr | H |
| Et | Et | Me | i-Pr | H |
| Et | Et | Et | i-Pr | H |
| Et | —(CH₂)₄— | | i-Pr | H |
| Et | Me | Me | t-Bu | H |
| E? | Et | Me | t-Bu | H |
| Et | Et | Et | t-Bu | H |

| | | | |
|---|---|---|---|
| Et | —(CH₂)₄— | t-Bu | H |

TABLE L4

| R¹ | R⁴ | R⁵ | R⁸ | R⁹ |
|---|---|---|---|---|
| i-Pr | Me | Me | H | H |
| i-Pr | Et | Me | H | H |
| i-Pr | Et | Et | H | H |
| i-Pr | —(CH₂)₄— | | H | H |
| i-Pr | Me | Me | Me | H |
| i-Pr | Et | Me | Me | H |
| i-Pr | Et | Et | Me | H |
| i-Pr | —(CH₂)₄— | | Me | H |
| i-Pr | Me | Me | Me | Me |
| i-Pr | Et | Me | Me | Me |
| i-Pr | Et | Et | Me | Me |
| i-Pr | —(CH₂)₄— | | Me | Me |
| i-Pr | Me | Me | Et | H |
| i-Pr | Et | Me | Et | H |
| i-Pr | Et | Et | Et | H |
| i-Pr | —(CH₂)₄— | | Et | H |
| i-Pr | Me | Me | Pr | H |
| i-Pr | Et | Me | Pr | H |
| i-Pr | Et | Et | Pr | H |
| i-Pr | —(CH₂)₄— | | Pr | H |
| i-Pr | Me | Me | i-Pr | H |
| i-Pr | Et | Me | i-Pr | H |
| i-Pr | Et | Et | i-Pr | H |
| i-Pr | —(CH₂)₄— | | i-Pr | H |
| i-Pr | Me | Me | t-Bu | H |
| i-Pr | Et | Me | t-Bu | H |
| i-Pr | Et | Et | t-Bu | H |

| | | | |
|---|---|---|---|
| i-Pr | —(CH₂)₄— | t-Bu | H |

A compound according to the compound (L-1), wherein Y represents an oxygen atom, p represents 1, Q represents a 2-fluorophenyl group, and the combination of R¹, R⁴, R⁵, R⁸, and R⁹ represents any one of the combinations shown in [Table L1] to [Table L4](hereinafter, referred to as a compound group SX2).

A compound according to the compound (L-1), wherein Y represents an oxygen atom, p represents 1, Q represents a 3-fluorophenyl group, and the combination of R¹, R⁴, R⁵, R⁸, and R⁹ represents any one of the combinations shown in [Table L1] to [Table L4](hereinafter, referred to as a compound group SX3).

A compound according to the compound (L-1), wherein Y represents an oxygen atom, p represents 1, Q represents a 4-fluorophenyl group, and the combination of R¹, R⁴, R⁵, R⁸, and R⁹ represents any one of the combinations shown in [Table L1] to [Table L4](hereinafter, referred to as a compound group SX4).

A compound according to the compound (L-1), wherein Y represents an oxygen atom, p represents 1, Q represents a 2-chlorophenyl group, and the combination of R¹, R⁴, R⁵, R⁸, and R⁹ represents any one of the combinations shown in [Table L1] to [Table L4](hereinafter, referred to as a compound group SX5).

A compound according to the compound (L-1), wherein Y represents an oxygen atom, p represents 1, Q represents a 3-chlorophenyl group, and the combination of R¹, R⁴, R⁵, R⁸, and R⁹ represents any one of the combinations shown in [Table L1] to [Table L4](hereinafter, referred to as a compound group SX6).

A compound according to the compound (L-1), wherein Y represents an oxygen atom, p represents 1, Q represents a 4-chlorophenyl group, and the combination of R¹, R⁴, R⁵, R⁸, and R⁹ represents any one of the combinations shown in [Table L1] to [Table L4](hereinafter, referred to as a compound group SX7).

A compound according to the compound (L-1), wherein Y represents an oxygen atom, p represents 1, Q represents a 2-bromophenyl group, and the combination of R¹, R⁴, R⁵, R⁸, and R⁹ represents any one of the combinations shown in [Table L1] to [Table L4](hereinafter, referred to as a compound group SX8).

A compound according to the compound (L-1), wherein Y represents an oxygen atom, p represents 1, Q represents a 3-bromophenyl group, and the combination of R¹, R⁴, R⁵, R⁸, and R⁹ represents any one of the combinations shown in [Table L1] to [Table L4](hereinafter, referred to as a compound group SX9).

A compound according to the compound (L-1), wherein Y represents an oxygen atom, p represents 1, Q represents a 4-bromophenyl group, and the combination of R¹, R⁴, R⁵, R⁸, and R⁹ represents any one of the combinations shown in [Table L1] to [Table L4](hereinafter, referred to as a compound group SX10).

A compound according to the compound (L-1), wherein Y represents an oxygen atom, p represents 1, Q represents a 2-iodophenyl group, and the combination of R¹, R⁴, R⁵, R⁸, and R⁹ represents any one of the combinations shown in [Table L1] to [Table L4](hereinafter, referred to as a compound group S11).

A compound according to the compound (L-1), wherein Y represents an oxygen atom, p represents 1, Q represents a 3-iodophenyl group, and the combination of R¹, R⁴, R⁵, R⁸, and R⁹ represents any one of the combinations shown in [Table L1] to [Table L4](hereinafter, referred to as a compound group SX12).

A compound according to the compound (L-1), wherein Y represents an oxygen atom, p represents 1, Q represents a 4-iodophenyl group, and the combination of R¹, R⁴, R⁵, R⁸, and R⁹ represents any one of the combinations shown in [Table L1] to [Table L4](hereinafter, referred to as a compound group SX13).

A compound according to the compound (L-1), wherein Y represents an oxygen atom, p represents 1, Q represents a 2-methylphenyl group, and the combination of R¹, R⁴, R⁵, R⁸, and R⁹ represents any one of the combinations shown in [Table L1] to [Table L4](hereinafter, referred to as a compound group SX14).

A compound according to the compound (L-1), wherein Y represents an oxygen atom, p represents 1, Q represents a 3-methylphenyl group, and the combination of R¹, R⁴, R⁵, R⁸, and R⁹ represents any one of the combinations shown in [Table L1] to [Table L4](hereinafter, referred to as a compound group SX15).

A compound according to the compound (L-1), wherein Y represents an oxygen atom, p represents 1, Q represents a 4-methylphenyl group, and the combination of R¹, R⁴, R⁵, R⁸, and R⁹ represents any one of the combinations shown in [Table L1] to [Table L4](hereinafter, referred to as a compound group SX16).

A compound according to the compound (L-1), wherein Y represents an oxygen atom, p represents 1, Q represents a 2-methoxyphenyl group, and the combination of R¹, R⁴, R⁵, R⁸, and R⁹ represents any one of the combinations shown in [Table L1] to [Table L4](hereinafter, referred to as a compound group SX17).

A compound according to the compound (L-1), wherein Y represents an oxygen atom, p represents 1, Q represents a 3-methoxyphenyl group, and the combination of R¹, R⁴, R⁵, R⁸, and R⁹ represents any one of the combinations shown in [Table L1] to [Table L4](hereinafter, referred to as a compound group SX18).

A compound according to the compound (L-1), wherein Y represents an oxygen atom, p represents 1, Q represents a 4-methoxyphenyl group, and the combination of R¹, R⁴, R⁵, R⁸, and R⁹ represents any one of the combinations shown in [Table L1] to [Table L4](hereinafter, referred to as a compound group SX19).

A compound according to the compound (L-1), wherein Y represents an oxygen atom, p represents 1, Q represents a 2-trifluoromethylphenyl group, and the combination of R¹, R⁴, R⁵, R⁸, and R⁹ represents any one of the combinations shown in [Table L1] to [Table L4](hereinafter, referred to as a compound group SX20).

A compound according to the compound (L-1), wherein Y represents an oxygen atom, p represents 1, Q represents a 3-trifluoromethylphenyl group, and the combination of R¹, R⁴, R⁵, R⁸, and R⁹ represents any one of the combinations shown in [Table L1] to [Table L4](hereinafter, referred to as a compound group SX21).

A compound according to the compound (L-1), wherein Y represents an oxygen atom, p represents 1, Q represents a 4-trifluoromethylphenyl group, and the combination of R¹, R⁴, R⁵, R⁸, and R⁹ represents any one of the combinations shown in [Table L1] to [Table L4](hereinafter, referred to as a compound group SX22).

A compound according to the compound (L-1), wherein Y represents an oxygen atom, p represents 1, Q represents a 2-trifluoromethoxyphenyl group, and the combination of R¹, R⁴, R⁵, R⁸, and R⁹ represents any one of the combinations shown in [Table L1] to [Table L4](hereinafter, referred to as a compound group S23).

A compound according to the compound (L-1), wherein Y represents an oxygen atom, p represents 1, Q represents a 3-trifluoromethoxyphenyl group, and the combination of R¹, R⁴, R⁵, R⁸, and R⁹ represents any one of the combinations shown in [Table L1] to [Table L4](hereinafter, referred to as a compound group SX24).

A compound according to the compound (L-1), wherein Y represents an oxygen atom, p represents 1, Q represents a 4-trifluoromethoxyphenyl group, and the combination of R¹, R⁴, R⁵, R⁸, and R⁹ represents any one of the combinations shown in [Table L1] to [Table L4](hereinafter, referred to as a compound group SX25).

A compound according to the compound (L-1), wherein Y represents an oxygen atom, p represents 1, Q represents a benzyl group, and the combination of R¹, R⁴, R⁵, R⁸, and R⁹ represents any one of the combinations shown in [Table L1] to [Table L4](hereinafter, referred to as a compound group SX26).

A compound according to the compound (L-1), wherein Y represents an oxygen atom, p represents 1, Q represents a 2-fluorobenzyl group, and the combination of R¹, R⁴, R⁵, R⁸, and R⁹ represents any one of the combinations shown in [Table L1] to [Table L4](hereinafter, referred to as a compound group SX27).

A compound according to the compound (L-1), wherein Y represents an oxygen atom, p represents 1, Q represents a 3-fluorobenzyl group, and the combination of R¹, R⁴, R⁵, R⁸, and R⁹ represents any one of the combinations shown in [Table L1] to [Table L4](hereinafter, referred to as a compound group SX28).

A compound according to the compound (L-1), wherein Y represents an oxygen atom, p represents 1, Q represents a 4-fluorobenzyl group, and the combination of R¹, R⁴, R⁵, R⁸, and R⁹ represents any one of the combinations shown in [Table L1] to [Table L4](hereinafter, referred to as a compound group SX29).

A compound according to the compound (L-1), wherein Y represents an oxygen atom, p represents 1, Q represents a 2-chlorobenzyl group, and the combination of R¹, R⁴, R⁵, R⁸, and R⁹ represents any one of the combinations shown in [Table L1] to [Table L4](hereinafter, referred to as a compound group SX30).

A compound according to the compound (L-1), wherein Y represents an oxygen atom, p represents 1, Q represents a 3-chlorobenzyl group, and the combination of R¹, R⁴, R⁵, R⁸, and R⁹ represents any one of the combinations shown in [Table L1] to [Table L4](hereinafter, referred to as a compound group SX31).

A compound according to the compound (L-1), wherein Y represents an oxygen atom, p represents 1, Q represents a 4-chlorobenzyl group, and the combination of R¹, R⁴, R⁵, R⁸, and R⁹ represents any one of the combinations shown in [Table L1] to [Table L4](hereinafter, referred to as a compound group SX32).

A compound according to the compound (L-1), wherein Y represents an oxygen atom, p represents 1, Q represents a 2-bromobenzyl group, and the combination of R¹, R⁴, R⁵, R⁸, and R⁹ represents any one of the combinations shown in [Table L1] to [Table L4](hereinafter, referred to as a compound group SX33).

A compound according to the compound (L-1), wherein Y represents an oxygen atom, p represents 1, Q represents a 3-bromobenzyl group, and the combination of R¹, R⁴, R⁵, R⁸, and R⁹ represents any one of the combinations shown in [Table L1] to [Table L4](hereinafter, referred to as a compound group SX34).

A compound according to the compound (L-1), wherein Y represents an oxygen atom, p represents 1, Q represents a 4-bromobenzyl group, and the combination of R¹, R⁴, R⁵, R⁸, and R⁹ represents any one of the combinations shown in [Table L1] to [Table L4](hereinafter, referred to as a compound group SX35).

A compound according to the compound (L-1), wherein Y represents an oxygen atom, p represents 1, Q represents a 2-iodobenzyl group, and the combination of R¹, R⁴, R⁵, R⁸, and R⁹ represents any one of the combinations shown in [Table L1] to [Table L4](hereinafter, referred to as a compound group S36).

A compound according to the compound (L-1), wherein Y represents an oxygen atom, p represents 1, Q represents a 3-iodobenzyl group, and the combination of R¹, R⁴, R⁵, R⁸, and R⁹ represents any one of the combinations shown in [Table L1] to [Table L4](hereinafter, referred to as a compound group SX37).

A compound according to the compound (L-1), wherein Y represents an oxygen atom, p represents 1, Q represents a 4-iodobenzyl group, and the combination of R¹, R⁴, R⁵, R⁸, and R⁹ represents any one of the combinations shown in [Table L1] to [Table L4](hereinafter, referred to as a compound group SX38).

A compound according to the compound (L-1), wherein Y represents an oxygen atom, p represents 1, Q represents a 2-methylbenzyl group, and the combination of R¹, R⁴, R⁵, R⁸, and R⁹ represents any one of the combinations shown in [Table L1] to [Table L4](hereinafter, referred to as a compound group SX39).

A compound according to the compound (L-1), wherein Y represents an oxygen atom, p represents 1, Q represents a 3-methylbenzyl group, and the combination of R¹, R⁴, R⁵, R⁸, and R⁹ represents any one of the combinations shown in [Table L1] to [Table L4](hereinafter, referred to as a compound group SX40).

A compound according to the compound (L-1), wherein Y represents an oxygen atom, p is 1, Q represents a 4-methylbenzyl group, and the combination of R¹, R⁴, R⁵, R⁸, and R⁹ represents any one of the combinations shown in [Table L1] to [Table L4](hereinafter, referred to as a compound group SX41).

A compound according to the compound (L-1), wherein Y represents an oxygen atom, p represents 1, Q represents a 2-methoxybenzyl group, and the combination of R¹, R⁴, R⁵, R⁸, and R⁹ represents any one of the combinations shown in [Table L1] to [Table L4](hereinafter, referred to as a compound group SX42).

A compound according to the compound (L-1), wherein Y represents an oxygen atom, p represents 1, Q represents a 3-methoxybenzyl group, and the combination of R¹, R⁴, R⁵, R⁸, and R⁹ represents any one of the combinations shown in [Table L1] to [Table L4](hereinafter, referred to as a compound group SX43).

A compound according to the compound (L-1), wherein Y represents an oxygen atom, p represents 1, Q represents a 4-methoxybenzyl group, and the combination of R¹, R⁴, R⁵, R⁸, and R⁹ represents any one of the combinations shown in [Table L1] to [Table L4](hereinafter, referred to as a compound group SX44).

A compound according to the compound (L-1), wherein Y represents an oxygen atom, p represents 1, Q represents a 2-trifluoromethylbenzyl group, and the combination of R¹, R⁴, R⁵, R⁸, and R⁹ represents any one of the combinations shown in [Table L1] to [Table L4](hereinafter, referred to as a compound group SX45).

A compound according to the compound (L-1), wherein Y represents an oxygen atom, p represents 1, Q represents a 3-trifluoromethylbenzyl group, and the combination of R¹, R⁴, R⁵, R⁸, and R⁹ represents any one of the combinations shown in [Table L1] to [Table L4](hereinafter, referred to as a compound group SX46).

A compound according to the compound (L-1), wherein Y represents an oxygen atom, p represents 1, Q represents a 4-trifluoromethylbenzyl group, and the combination of R¹, R⁴, R⁵, R⁸, and R⁹ represents any one of the combinations shown in [Table L1] to [Table L4](hereinafter, referred to as a compound group SX47).

A compound according to the compound (L-1), wherein Y represents an oxygen atom, p represents 1, Q represents a 2-trifluoromethoxybenzyl group, and the combination of R¹, R⁴, R⁵, R⁸, and R⁹ represents any one of the combinations shown in [Table L1] to [Table L4](hereinafter, referred to as a compound group S48).

A compound according to the compound (L-1), wherein Y represents an oxygen atom, p represents 1, Q represents a 3-trifluoromethoxybenzyl group, and the combination of R¹, R⁴, R⁵, R⁸, and R⁹ represents any one of the combinations shown in [Table L1] to [Table L4](hereinafter, referred to as a compound group SX49).

A compound according to the compound (L-1), wherein Y represents an oxygen atom, p represents 1, Q represents a 4-trifluoromethoxybenzyl group, and the combination of R¹, R⁴, R⁵, R⁸, and R⁹ represents any one of the combinations shown in [Table L1] to [Table L4](hereinafter, referred to as a compound group SX50).

A compound according to the compound (L-1), wherein Y represents an oxygen atom, p represents 1, Q represents a 2-naphthyl group, and the combination of R¹, R⁴, R⁵, R⁸, and R⁹ represents any one of the combinations shown in [Table L1] to [Table L4](hereinafter, referred to as a compound group SX51).

A compound according to the compound (L-1), wherein Y represents an oxygen atom, p represents 1, Q represents a 2-naphthylmethyl group, and the combination of R¹, R⁴, R⁵, R⁸, and R⁹ represents any one of the combinations shown in [Table L1] to [Table L4](hereinafter, referred to as a compound group SX52).

A compound according to the compound (L-1), wherein Y represents an oxygen atom, p represents 1, Q represents a 5-chloro-1-naphthyl group, and the combination of R¹, R⁴, R⁵, R⁸, and R⁹ represents any one of the combinations shown in [Table L1] to [Table L4](hereinafter, referred to as a compound group SX53).

A compound according to the compound (L-1), wherein Y represents an oxygen atom, p represents 1, Q represents a 6-chloro-1-naphthyl group, and the combination of R¹, R⁴, R⁵, R⁸, and R⁹ represents any one of the combinations shown in [Table L1] to [Table L4](hereinafter, referred to as a compound group SX54).

A compound according to the compound (L-1), wherein Y represents an oxygen atom, p represents 1, Q represents a 5,6-dichloro-1-naphthyl group, and the combination of R¹, R⁴, R⁵, R⁸, and R⁹ represents any one of the combinations shown in [Table L1] to [Table L4](hereinafter, referred to as a compound group SX55).

A compound according to the compound (L-1), wherein Y represents an oxygen atom, p represents 1, Q represents a 5-methyl-1-naphthyl group, and the combination of R¹, R⁴, R⁵, R⁸, and R⁹ represents any one of the combinations shown in [Table L1] to [Table L4](hereinafter, referred to as a compound group SX56).

A compound according to the compound (L-1), wherein Y represents an oxygen atom, p represents 1, Q represents a 6-methyl-1-naphthyl group, and the combination of R¹, R⁴, R⁵, R⁸, and R⁹ represents any one of the combinations shown in [Table L1] to [Table L4](hereinafter, referred to as a compound group SX57).

A compound according to the compound (L-1), wherein Y represents an oxygen atom, p represents 1, Q represents a 5,6-dimethyl-1-naphthyl group, and the combination of R¹, R⁴, R⁵, R⁸, and R⁹ represents any one of the combinations shown in [Table L1] to [Table L4](hereinafter, referred to as a compound group SX58).

A compound according to the compound (L-1), wherein Y represents an oxygen atom, p represents 1, Q represents a 3-phenyl-4-fluorophenyl group, and the combination of R¹, R⁴, R⁵, R⁸, and R⁹ represents any one of the combinations shown in [Table L1] to [Table L4](hereinafter, referred to as a compound group SX59).

A compound according to the compound (L-1), wherein Y represents an oxygen atom, p represents 1, Q represents a 3-(2-fluorophenyl)-4-fluorophenyl group, and the combination of R¹, R⁴, R⁵, R⁸, and R⁹ represents any one of the combinations shown in [Table L1] to [Table L4](hereinafter, referred to as a compound group SX60).

A compound according to the compound (L-1), wherein Y represents an oxygen atom, p represents 1, Q represents a 3-(3-fluorophenyl)-4-fluorophenyl group, and the combination of R¹, R⁴, R⁵, R⁸, and R⁹ represents any one of the combinations shown in [Table L1] to [Table L4](hereinafter, referred to as a compound group SX61).

A compound according to the compound (L-1), wherein Y represents an oxygen atom, p represents 1, Q represents a 3-(4-fluorophenyl)-4-fluorophenyl group, and the combination of R¹, R⁴, R⁵, R⁸, and R⁹ represents any one of the combinations shown in [Table L1] to [Table L4](hereinafter, referred to as a compound group SX62).

A compound according to the compound (L-1), wherein Y represents an oxygen atom, p represents 1, Q represents a 3-(2-chlorophenyl)-4-fluorophenyl group, and the combination of R¹, R⁴, R⁵, R⁸, and R⁹ represents any one of the combinations shown in [Table L1] to [Table L4](hereinafter, referred to as a compound group SX63).

A compound according to the compound (L-1), wherein Y represents an oxygen atom, p represents 1, Q represents a 3-(3-chlorophenyl)-4-fluorophenyl group, and the combination of R¹, R⁴, R⁵, R⁸, and R⁹ represents any one of the combinations shown in [Table L1] to [Table L4](hereinafter, referred to as a compound group SX64).

A compound according to the compound (L-1), wherein Y represents an oxygen atom, p represents 1, Q represents a 3-(4-chlorophenyl)-4-fluorophenyl group, and the combination of R¹, R⁴, R⁵, R⁸, and R⁹ represents any one of the combinations shown in [Table L1] to [Table L4](hereinafter, referred to as a compound group SX65).

A compound according to the compound (L-1), wherein Y represents an oxygen atom, p represents 1, Q represents a 3-(2-methylphenyl)-4-fluorophenyl group, and the combination of R¹, R⁴, R⁵, R⁸, and R⁹ represents any one of the combinations shown in [Table L1] to [Table L4](hereinafter, referred to as a compound group SX66).

A compound according to the compound (L-1), wherein Y represents an oxygen atom, p represents 1, Q represents a 3-(3-methylphenyl)-4-fluorophenyl group, and the combination of R¹, R⁴, R⁵, R⁸, and R⁹ represents any one of the combinations shown in [Table L1] to [Table L4](hereinafter, referred to as a compound group SX67).

A compound according to the compound (L-1), wherein Y represents an oxygen atom, p represents 1, Q represents a 3-(4-methylphenyl)-4-fluorophenyl group, and the combination of R¹, R⁴, R⁵, R⁸, and R⁹ represents any one of the combinations shown in [Table L1] to [Table L4](hereinafter, referred to as a compound group SX68).

A compound according to the compound (L-1), wherein Y represents an oxygen atom, p represents 1, Q represents a 3-(2-methoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX69).

A compound according to the compound (L-1), wherein Y represents an oxygen atom, p represents 1, Q represents a 3-(3-methoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX70).

A compound according to the compound (L-1), wherein Y represents an oxygen atom, p represents 1, Q represents a 3-(4-methoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX71).

A compound according to the compound (L-1), wherein Y represents an oxygen atom, p represents 1, Q represents a 3-(2-trifluoromethylphenyl)-4-fluorophenyl group, and the combination of R¹, R⁴, R⁵, R⁸, and R⁹ represents any one of the combinations shown in [Table L1] to [Table L4](hereinafter, referred to as a compound group SX72).

A compound according to the compound (L-1), wherein Y represents an oxygen atom, p represents 1, Q represents a 3-(3-trifluoromethylphenyl)-4-fluorophenyl group, and the combination of R¹, R⁴, R⁵, R⁸, and R⁹ represents any one of the combinations shown in [Table L1] to [Table L4](hereinafter, referred to as a compound group SX73).

A compound according to the compound (L-1), wherein Y represents an oxygen atom, p represents 1, Q represents a 3-(4-trifluoromethylphenyl)-4-fluorophenyl group, and the combination of R¹, R⁴, R⁵, R⁸, and R⁹ represents any one of the combinations shown in [Table L1] to [Table L4](hereinafter, referred to as a compound group SX74).

A compound according to the compound (L-1), wherein Y represents an oxygen atom, p represents 1, Q represents a 3-(2-trifluoromethoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX75).

A compound according to the compound (L-1), wherein Y represents an oxygen atom, p represents 1, Q represents a 3-(3-trifluoromethoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX76).

A compound according to the compound (L-1), wherein Y represents an oxygen atom, p represents 1, Q represents a 3-(4-trifluoromethoxyphenyl)-4-fluorophenyl group, and the combination of R¹, R⁴, R⁵, R⁸, and R⁹ represents any one of the combinations shown in [Table L1] to [Table L4](hereinafter, referred to as a compound group SX77).

A compound according to the compound (L-1), wherein Y represents an oxygen atom, p represents 1, Q represents a 3-fluoro-5-phenylphenyl group, and the combination of R¹, R⁴, R⁵, R⁸, and R⁹ represents any one of the combinations shown in [Table L1] to [Table L4](hereinafter, referred to as a compound group SX78).

A compound according to the compound (L-1), wherein Y represents an oxygen atom, p represents 1, Q represents a 3-fluoro-5-(2-fluorophenyl)phenyl group, and the combination of R¹, R⁴, R⁵, R⁸, and R⁹ represents any one of the combinations shown in [Table L1] to [Table L4](hereinafter, referred to as a compound group SX79).

A compound according to the compound (L-1), wherein Y represents an oxygen atom, p represents 1, Q represents a 3-fluoro-5-(3-fluorophenyl)phenyl group, and the combination of R¹, R⁴, R⁵, R⁸, and R⁹ represents any one of the combinations shown in [Table L1] to [Table L4](hereinafter, referred to as a compound group SX80).

A compound according to the compound (L-1), wherein Y represents an oxygen atom, p represents 1, Q represents a 3-fluoro-5-(4-fluorophenyl)phenyl group, and the combination of R¹, R⁴, R⁵, R⁸, and R⁹ represents any one of the combinations shown in [Table L1] to [Table L4](hereinafter, referred to as a compound group SX81).

A compound according to the compound (L-1), wherein Y represents an oxygen atom, p represents 1, Q represents a 3-fluoro-5-(2-chlorophenyl)phenyl group, and the combination of R¹, R⁴, R⁵, R⁸, and R⁹ represents any one of the combinations shown in [Table L1] to [Table L4](hereinafter, referred to as a compound group SX82).

A compound according to the compound (L-1), wherein Y represents an oxygen atom, p represents 1, Q represents a 3-fluoro-5-(3-chlorophenyl)phenyl group, and the combination of R¹, R⁴, R⁵, R⁸, and R⁹ represents any one of the combinations shown in [Table L1] to [Table L4](hereinafter, referred to as a compound group SX83).

A compound according to the compound (L-1), wherein Y represents an oxygen atom, p represents 1, Q represents a 3-fluoro-5-(4-chlorophenyl)phenyl group, and the combination of R¹, R⁴, R⁵, R⁸, and R⁹ represents any one of the combinations shown in [Table L1] to [Table L4](hereinafter, referred to as a compound group SX84).

A compound according to the compound (L-1), wherein Y represents an oxygen atom, p represents 1, Q represents a 3-fluoro-5-(2-methylphenyl)phenyl group, and the combination of R¹, R⁴, R⁵, R⁸, and R⁹ represents any one of the combinations shown in [Table L1] to [Table L4](hereinafter, referred to as a compound group SX85).

A compound according to the compound (L-1), wherein Y represents an oxygen atom, p represents 1, Q represents a 3-fluoro-5-(3-methylphenyl)phenyl group, and the combination of R¹, R⁴, R⁵, R⁸, and R⁹ represents any one of the combinations shown in [Table L1] to [Table L4](hereinafter, referred to as a compound group SX86).

A compound according to the compound (L-1), wherein Y represents an oxygen atom, p represents 1, Q represents a 4-fluoro-5-(3-methylphenyl)phenyl group, and the combination of R¹, R⁴, R⁵, R⁸, and R⁹ represents any one of the combinations shown in [Table L1] to [Table L4](hereinafter, referred to as a compound group SX87).

A compound according to the compound (L-1), wherein Y represents an oxygen atom, p represents 1, Q represents a 3-fluoro-5-(2-methylphenyl)phenyl group, and the combination of R¹, R⁴, R⁵, R⁸, and R⁹ represents any one of the combinations shown in [Table L1] to [Table L4](hereinafter, referred to as a compound group SX88).

A compound according to the compound (L-1), wherein Y represents an oxygen atom, p represents 1, Q represents a 3-fluoro-5-(3-methylphenyl)phenyl group, and the combination of R¹, R⁴, R⁵, R⁸, and R⁹ represents any one of the combinations shown in [Table L1] to [Table L4](hereinafter, referred to as a compound group SX89).

A compound according to the compound (L-1), wherein Y represents an oxygen atom, p represents 1, Q represents a 3-fluoro-5-(4-methylphenyl)phenyl group, and the combination of R¹, R⁴, R⁵, R⁸, and R⁹ represents any one of the combinations shown in [Table L1] to [Table L4](hereinafter, referred to as a compound group SX90).

A compound according to the compound (L-1), wherein Y represents an oxygen atom, p represents 1, Q represents a 3-fluoro-5-(2-trifluoromethylphenyl)phenyl group, and the combination of R¹, R⁴, R⁵, R⁸, and R⁹ represents any one of the combinations shown in [Table L1] to [Table L4](hereinafter, referred to as a compound group SX91).

A compound according to the compound (L-1), wherein Y represents an oxygen atom, p represents 1, Q represents a 3-fluoro-5-(3-trifluoromethylphenyl)phenyl group, and the combination of R¹, R⁴, R⁵, R⁸, and R⁹ represents any one of the combinations shown in [Table L1] to [Table L4](hereinafter, referred to as a compound group SX92).

A compound according to the compound (L-1), wherein Y represents an oxygen atom, p represents 1, Q represents a 3-fluoro-5-(4-trifluoromethylphenyl)phenyl group, and the combination of R¹, R⁴, R⁵, R⁸, and R⁹ represents any one of the combinations shown in [Table L1] to [Table L4](hereinafter, referred to as a compound group SX93).

A compound according to the compound (L-1), wherein Y represents an oxygen atom, p represents 1, Q represents a 3-fluoro-5-(2-trifluoromethoxyphenyl)phenyl group, and the combination of R¹, R⁴, R⁵, R⁸, and R⁹ represents any one of the combinations shown in [Table L1] to [Table L4](hereinafter, referred to as a compound group SX94).

A compound according to the compound (L-1), wherein Y represents an oxygen atom, p represents 1, Q represents a 3-fluoro-5-(3-trifluoromethoxyphenyl)phenyl group, and the combination of R¹, R⁴, R⁵, R⁸, and R⁹ represents any one of the combinations shown in [Table L1] to [Table L4](hereinafter, referred to as a compound group SX95).

A compound according to the compound (L-1), wherein Y represents an oxygen atom, p represents 1, Q represents a 3-fluoro-5-(4-trifluoromethoxyphenyl)phenyl group, and the combination of R¹, R⁴, R⁵, R⁸, and R⁹ represents any one of the combinations shown in [Table L1] to [Table L4](hereinafter, referred to as a compound group SX96).

A compound according to the compound (L-1), wherein Y represents a single bond, p represents 0, Q represents a benzyl group, and the combination of R¹, R⁴, R⁵, R⁸, and R⁹ represents any one of the combinations shown in [Table L1] to [Table L4](hereinafter, referred to as a compound group SX97).

A compound according to the compound (L-1), wherein Y represents a single bond, p represents 0, Q represents a 2-fluorobenzyl group, and the combination of R¹, R⁴, R⁵, R⁸, and R⁹ represents any one of the combinations shown in [Table L1] to [Table L4](hereinafter, referred to as a compound group SX98).

A compound according to the compound (L-1), wherein Y represents a single bond, p represents 0, Q represents a 3-fluorobenzyl group, and the combination of R¹, R⁴, R⁵, R⁸, and R⁹ represents any one of the combinations shown in [Table L1] to [Table L4](hereinafter, referred to as a compound group SX99).

A compound according to the compound (L-1), wherein Y represents a single bond, p represents 0, Q represents a 4-fluorobenzyl group, and the combination of R¹, R⁴, R⁵, R⁸, and R⁹ represents any one of the combinations shown in [Table L1] to [Table L4](hereinafter, referred to as a compound group SX100).

A compound according to the compound (L-1), wherein Y represents a single bond, p represents 0, Q represents a 2-chlorobenzyl group, and the combination of R¹, R⁴, R⁵, R⁸, and R⁹ represents any one of the combinations shown in [Table L1] to [Table L4](hereinafter, referred to as a compound group SX101).

A compound according to the compound (L-1), wherein Y represents a single bond, p represents 0, Q represents a 3-chlorobenzyl group, and the combination of R¹, R⁴, R⁵, R⁸, and R⁹ represents any one of the combinations shown in [Table L1] to [Table L4](hereinafter, referred to as a compound group SX102).

A compound according to the compound (L-1), wherein Y represents a single bond, p represents 0, Q represents a 4-chlorobenzyl group, and the combination of R¹, R⁴, R⁵, R⁸, and R⁹ represents any one of the combinations shown in [Table L1] to [Table L4](hereinafter, referred to as a compound group SX103).

A compound according to the compound (L-1), wherein Y represents a single bond, p represents 0, Q represents a 2-methylbenzyl group, and the combination of R¹, R⁴, R⁵, R⁸, and R⁹ represents any one of the combinations shown in [Table L1] to [Table L4](hereinafter, referred to as a compound group 104).

A compound according to the compound (L-1), wherein Y represents a single bond, p represents 0, Q represents a 3-methylbenzyl group, and the combination of R¹, R⁴, R⁵, R⁸, and R⁹ represents any one of the combinations shown in [Table L1] to [Table L4](hereinafter, referred to as a compound group SX105).

A compound according to the compound (L-1), wherein Y represents a single bond, p represents 0, Q represents a 4-methylbenzyl group, and the combination of R¹, R⁴, R⁵, R⁸, and R⁹ represents any one of the combinations shown in [Table L1] to [Table L4](hereinafter, referred to as a compound group SX106).

A compound according to the compound (L-1), wherein Y represents a single bond, p represents 0, Q represents a 2-methoxybenzyl group, and the combination of R¹, R⁴, R⁵, R⁸, and R⁹ represents any one of the combinations shown in [Table L1] to [Table L4](hereinafter, referred to as a compound group SX107).

A compound according to the compound (L-1), wherein Y represents a single bond, p represents 0, Q represents a 3-methoxybenzyl group, and the combination of R¹, R⁴, R⁵, R⁸, and R⁹ represents any one of the combinations shown in [Table L1] to [Table L4](hereinafter, referred to as a compound group SX108).

A compound according to the compound (L-1), wherein Y represents a single bond, p represents 0, Q represents a 4-methoxybenzyl group, and the combination of R¹, R⁴, R⁵, R⁸, and R⁹ represents any one of the combinations shown in [Table L1] to [Table L4](hereinafter, referred to as a compound group SX109).

A compound according to the compound (L-1), wherein Y represents a single bond, p represents 0, Q represents a 4-phenylbenzyl group, and the combination of R¹, R⁴, R⁵, R⁸, and R⁹ represents any one of the combinations shown in [Table L1] to [Table L4](hereinafter, referred to as a compound group SX110).

A compound according to the compound (L-1), wherein Y represents a single bond, p represents 0, Q represents a 4-(2-fluorophenyl)benzyl group, and the combination of R¹, R⁴, R⁵, R⁸, and R⁹ represents any one of the combinations shown in [Table L1] to [Table L4](hereinafter, referred to as a compound group SX111).

A compound according to the compound (L-1), wherein Y represents a single bond, p represents 0, Q represents a 4-(3-fluorophenyl)benzyl group, and the combination of R¹, R⁴, R⁵, R⁸, and R⁹ represents any one of the combinations shown in [Table L1] to [Table L4](hereinafter, referred to as a compound group SX112).

A compound according to the compound (L-1), wherein Y represents a single bond, p represents 0, Q represents a 4-(4-fluorophenyl)benzyl group, and the combination of R¹, R⁴, R⁵, R⁸, and R⁹ represents any one of the combinations shown in [Table L1] to [Table L4](hereinafter, referred to as a compound group SX113).

A compound according to the compound (L-1), wherein Y represents a single bond, p represents 0, Q represents a 4-(2-chlorophenyl)benzyl group, and the combination of R¹, R⁴, R⁵, R⁸, and R⁹ represents any one of the combinations shown in [Table L1] to [Table L4](hereinafter, referred to as a compound group SX114).

A compound according to the compound (L-1), wherein Y represents a single bond, p represents 0, Q represents a 4-(3-chlorophenyl)benzyl group, and the combination of R¹, R⁴, R⁵, R⁸, and R⁹ represents any one of the combinations shown in [Table L1] to [Table L4](hereinafter, referred to as a compound group SX115).

A compound according to the compound (L-1), wherein Y represents a single bond, p represents 0, Q represents a 4-(4-chlorophenyl)benzyl group, and the combination of R¹, R⁴, R⁵, R⁸, and R⁹ represents any one of the combinations shown in [Table L1] to [Table L4](hereinafter, referred to as a compound group SX116).

A compound according to the compound (L-1), wherein Y represents a single bond, p represents 0, Q represents a 4-(2-methylphenyl)benzyl group, and the combination of R¹, R⁴, R⁵, R⁸, and R⁹ represents any one of the combinations shown in [Table L1] to [Table L4](hereinafter, referred to as a compound group SX117).

A compound according to the compound (L-1), wherein Y represents a single bond, p represents 0, Q represents a 4-(3-methylphenyl)benzyl group, and the combination of R¹, R⁴, R⁵, R⁸, and R⁹ represents any one of the combinations shown in [Table L1] to [Table L4](hereinafter, referred to as a compound group SX118).

A compound according to the compound (L-1), wherein Y represents a single bond, p represents 0, Q represents a 4-(4-methylphenyl)benzyl group, and the combination of R¹, R⁴, R⁵, R⁸, and R⁹ represents any one of the combinations shown in [Table L1] to [Table L4](hereinafter, referred to as a compound group SX119).

A compound according to the compound (L-1), wherein Y represents a single bond, p represents 0, Q represents a 4-(2-methylphenyl)benzyl group, and the combination of R¹, R⁴, R⁵, R⁸, and R⁹ represents any one of the combinations shown in [Table L1] to [Table L4](hereinafter, referred to as a compound group SX120).

A compound according to the compound (L-1), wherein Y represents a single bond, p represents 0, Q represents a 4-(3-methylphenyl)benzyl group, and the combination of R¹, R⁴, R⁵, R⁸, and R⁹ represents any one of the combinations shown in [Table L1] to [Table L4](hereinafter, referred to as a compound group SX121).

A compound according to the compound (L-1), wherein Y represents a single bond, p represents 0, Q represents a 4-(4-methylphenyl)benzyl group, and the combination of R¹, R⁴, R⁵, R⁸, and R⁹ represents any one of the combinations shown in [Table L1] to [Table L4](hereinafter, referred to as a compound group SX122).

A compound according to the compound (L-1), wherein Y represents a single bond, p represents 0, Q represents a 4-(2-trifluoromethylphenyl)benzyl group, and the combination of R¹, R⁴, R⁵, R⁸, and R⁹ represents any one of the combinations shown in [Table L1] to [Table L4](hereinafter, referred to as a compound group SX123).

A compound according to the compound (L-1), wherein Y represents a single bond, p represents 0, Q represents a 4-(3-trifluoromethylphenyl)benzyl group, and the combination of R¹, R⁴, R⁵, R⁸, and R⁹ represents any one of the combinations shown in [Table L1] to [Table L4](hereinafter, referred to as a compound group SX124).

A compound according to the compound (L-1), wherein Y represents a single bond, p represents 0, Q represents a 4-(4-trifluoromethylphenyl)benzyl group, and the combination of R¹, R⁴, R⁵, R⁸, and R⁹ represents any one of the combinations shown in [Table L1] to [Table L4](hereinafter, referred to as a compound group SX125).

A compound according to the compound (L-1), wherein Y represents a single bond, p represents 0, Q represents a 4-(2-trifluoromethoxyphenyl)benzyl group, and the combination of R¹, R⁴, R⁵, R⁸, and R⁹ represents any one of the combinations shown in [Table L1] to [Table L4](hereinafter, referred to as a compound group SX126).

A compound according to the compound (L-1), wherein Y represents a single bond, p represents 0, Q represents a 4-(3-trifluoromethoxyphenyl)benzyl group, and the combination of R¹, R⁴, R⁵, R⁸, and R⁹ represents any one of the combinations shown in [Table L1] to [Table L4](hereinafter, referred to as a compound group SX127).

A compound according to the compound (L-1), wherein Y represents a single bond, p represents 0, Q represents a 4-(4-trifluoromethoxyphenyl)benzyl group, and the combination of R¹, R⁴, R⁵, R⁸, and R⁹ represents any one of the combinations shown in [Table L1] to [Table L4](hereinafter, referred to as a compound group SX128).

A compound according to the compound (L-1), wherein Y represents a single bond, p represents 0, Q represents a 4-(1-pyrrolyl)benzyl group, and the combination of R¹, R⁴, R⁵, R⁸, and R⁹ represents any one of the combinations shown in [Table L1] to [Table L4](hereinafter, referred to as a compound group SX129).

A compound according to the compound (L-1), wherein Y represents a single bond, p represents 0, Q represents a 4-(1-pyrazolyl)benzyl group, and the combination of R¹, R⁴, R⁵, R⁸, and R⁹ represents any one of the combinations shown in [Table L1] to [Table L4](hereinafter, referred to as a compound group SX130).

A compound according to the compound (L-1), wherein Y represents a single bond, p represents 0, Q represents a 4-(1-imidazolyl)benzyl group, and the combination of R¹, R⁴, R⁵, R⁸, and R⁹ represents any one of the combinations shown in [Table L1] to [Table L4](hereinafter, referred to as a compound group SX131).

A compound according to the compound (L-1), wherein Y represents a single bond, p represents 0, Q represents a 4-(1,2,3-triazol-1-yl)benzyl group, and the combination of R¹, R⁴, R⁵, R⁸, and R⁹ represents any one of the combinations shown in [Table L1] to [Table L4](hereinafter, referred to as a compound group SX132).

A compound according to the compound (L-1), wherein Y represents a single bond, p represents 0, Q represents a 4-(1,2,3-triazol-2-yl)benzyl group, and the combination of R¹, R⁴, R⁵, R⁸, and R⁹ represents any one of the combinations shown in [Table L1] to [Table L4](hereinafter, referred to as a compound group SX133).

A compound according to the compound (L-1), wherein Y represents a single bond, p represents 0, Q represents a hydrogen atom, and the combination of R¹, R⁴, R⁵, R⁸, and R⁹ represents any one of the combinations shown in [Table L1] to [Table L4](hereinafter, referred to as a compound group SX134).

A compound according to the compound (L-1), wherein Y represents a single bond, p represents 0, Q represents a methyl group, and the combination of R¹, R⁴, R⁵, R⁸, and R⁹ represents any one of the combinations shown in [Table L1] to [Table L4](hereinafter, referred to as a compound group SX135).

A compound according to the compound (L-1), wherein Y represents a single bond, p represents 0, Q represents an ethyl group, and the combination of R¹, R⁴, R⁵, R³, and R⁹ represents any one of the combinations shown in [Table L1] to [Table L4](hereinafter, referred to as a compound group SX136).

A compound according to the compound (L-1), wherein Y represents a single bond, p represents 0, Q represents a propyl group, and the combination of R¹, R⁴, R⁵, R³, and R⁹ represents any one of the combinations shown in [Table L1] to [Table L4](hereinafter, referred to as a compound group SX137).

A compound according to the compound (L-1), wherein Y represents a single bond, p represents 0, Q represents a butyl group, and the combination of R¹, R⁴, R⁵, R³, and R⁹ represents any one of the combinations shown in [Table L1] to [Table L4](hereinafter, referred to as a compound group SX138).

A compound according to the compound (L-1), wherein Y represents a single bond, p represents 0, Q represents a C5H₁₁ group, and the combination of R¹, R⁴, R⁵, R³, and R⁹ represents any one of the combinations shown in [Table L1] to [Table L4](hereinafter, referred to as a compound group SX139).

A compound according to the compound (L-1), wherein Y represents a single bond, p represents 0, Q represents a C6H₁₃ group, and the combination of R¹, R⁴, R⁵, R³, and R⁹ represents any one of the combinations shown in [Table L1] to [Table L4](hereinafter, referred to as a compound group SX140).

A compound according to the compound (L-1), wherein Y represents a single bond, p represents 0, Q represents a C7H₁₅ group, and the combination of R¹, R⁴, R⁵, R⁸, and R⁹ represents any one of the combinations shown in [Table L1] to [Table L4](hereinafter, referred to as a compound group SX141).

A compound according to the compound (L-1), wherein Y represents a single bond, p represents 0, Q represents a C₈H₁₇ group, and the combination of R¹, R⁴, R⁵, R⁸, and R⁹ represents any one of the combinations shown in [Table L1] to [Table L4](hereinafter, referred to as a compound group SX142).

A compound according to the compound (L-1), wherein Y represents a single bond, p represents 0, Q represents a CH₂-benzofuran-2-yl group, and the combination of R¹, R⁴, R⁵, R⁸, and R⁹ represents any one of the combinations shown in [Table L1] to [Table L4](hereinafter, referred to as a compound group SX143).

A compound according to the compound (L-1), wherein Y represents a single bond, p represents 0, Q represents a CH₂-3-methylbenzofuran-2-yl group, and the combination of R¹, R⁴, R⁵, R⁸, and R⁹ represents any one of the combinations shown in [Table L1] to [Table L4](hereinafter, referred to as a compound group SX144).

A compound according to the compound (L-1), wherein Y represents a single bond, p represents 0, Q represents a CH₂-4-methylbenzofuran-2-yl group, and the combination of R¹, R⁴, R⁵, R⁸, and R⁹ represents any one of the combinations shown in [Table L1] to [Table L4](hereinafter, referred to as a compound group SX145).

A compound according to the compound (L-1), wherein Y represents a single bond, p represents 0, Q represents a CH₂-5-methylbenzofuran-2-yl group, and the combination of R¹, R⁴, R⁵, R⁸, and R⁹ represents any one of the combinations shown in [Table L1] to [Table L4](hereinafter, referred to as a compound group SX146).

A compound according to the compound (L-1), wherein Y represents a single bond, p represents 0, Q represents a CH₂-6-methylbenzofuran-2-yl group, and the combination of R¹, R⁴, R⁵, R⁸, and R⁹ represents any one of the combinations shown in [Table L1] to [Table L4](hereinafter, referred to as a compound group SX147).

A compound according to the compound (L-1), wherein Y represents a single bond, p represents 0, Q represents a CH₂-7-methylbenzofuran-2-yl group, and the combination of R¹, R⁴, R⁵, R⁸, and R⁹ represents any one of the combinations shown in [Table L1] to [Table L4](hereinafter, referred to as a compound group SX148).

A compound according to the compound (L-1), wherein Y represents a single bond, p represents 0, Q represents a CH₂-3-fluorobenzofuran-2-yl group, and the combination of R¹, R⁴, R⁵, R⁸, and R⁹ represents any one of the combinations shown in [Table L1] to [Table L4](hereinafter, referred to as a compound group SX149).

A compound according to the compound (L-1), wherein Y represents a single bond, p represents 0, Q represents a CH₂-4-fluorobenzofuran-2-yl group, and the combination of R¹, R⁴, R⁵, R⁸, and R⁹ represents any one of the combinations shown in [Table L1] to [Table L4](hereinafter, referred to as a compound group SX150).

A compound according to the compound (L-1), wherein Y represents a single bond, p represents 0, Q represents a CH₂-5-fluorobenzofuran-2-yl group, and the combination of R¹, R⁴, R⁵, R⁸, and R⁹ represents any one of the combinations shown in [Table L1] to [Table L4](hereinafter, referred to as a compound group SX151).

A compound according to the compound (L-1), wherein Y represents a single bond, p represents 0, Q represents a CH₂-6-fluorobenzofuran-2-yl group, and the combination of R¹, R⁴, R⁵, R⁸, and R⁹ represents any one of the combinations shown in [Table L1] to [Table L4](hereinafter, referred to as a compound group SX152).

A compound according to the compound (L-1), wherein Y represents a single bond, p represents 0, Q represents a CH₂-7-fluorobenzofuran-2-yl group, and the combination of R¹, R⁴, R⁵, R⁸, and R⁹ represents any one of the combinations shown in [Table L1] to [Table L4](hereinafter, referred to as a compound group SX153).

A compound according to the compound (L-1), wherein Y represents a single bond, p represents 0, Q represents a CH₂-3-chlorobenzofuran-2-yl group, and the combination of R¹, R⁴, R⁵, R⁸, and R⁹ represents any one of the combinations shown in [Table L1] to [Table L4](hereinafter, referred to as a compound group SX154).

A compound according to the compound (L-1), wherein Y represents a single bond, p represents 0, Q represents a CH₂-4-chlorobenzofuran-2-yl group, and the combination of R¹, R⁴, R⁵, R⁸, and R⁹ represents any one of the combinations shown in [Table L1] to [Table L4](hereinafter, referred to as a compound group SX155).

A compound according to the compound (L-1), wherein Y represents a single bond, p represents 0, Q represents a CH₂-5-chlorobenzofuran-2-yl group, and the combination of R¹, R⁴, R⁵, R⁸, and R⁹ represents any one of the combinations shown in [Table L1] to [Table L4](hereinafter, referred to as a compound group SX156).

A compound according to the compound (L-1), wherein Y represents a single bond, p represents 0, Q represents a CH₂-6-chlorobenzofuran-2-yl group, and the combination of R¹, R⁴, R⁵, R⁸, and R⁹ represents any one of the combinations shown in [Table L1] to [Table L4](hereinafter, referred to as a compound group SX157).

A compound according to the compound (L-1), wherein Y represents a single bond, p represents 0, Q represents a CH₂-7-chlorobenzofuran-2-yl group, and the combination of R¹, R⁴, R⁵, R⁸, and R⁹ represents any one of the combinations shown in [Table L1] to [Table L4](hereinafter, referred to as a compound group SX158).

A compound according to the compound (L-1), wherein Y represents a single bond, p represents 0, Q represents a CH₂-3-methoxybenzofuran-2-yl group, and the combination of R¹, R⁴, R⁵, R⁸, and R⁹ represents any one of the combinations shown in [Table L1] to [Table L4](hereinafter, referred to as a compound group SX159).

A compound according to the compound (L-1), wherein Y represents a single bond, p represents 0, Q represents a CH₂-4-methoxybenzofuran-2-yl group, and the combination of R¹, R⁴, R⁵, R⁸, and R⁹ represents any one of the combinations shown in [Table L1] to [Table L4](hereinafter, referred to as a compound group SX160).

A compound according to the compound (L-1), wherein Y represents a single bond, p represents 0, Q represents a CH₂-5-methoxybenzofuran-2-yl group, and the combination of R¹, R⁴, R⁵, R⁸, and R⁹ represents any one of the combinations shown in [Table L1] to [Table L4](hereinafter, referred to as a compound group SX161).

A compound according to the compound (L-1), wherein Y represents a single bond, p represents 0, Q represents a CH₂-6-methoxybenzofuran-2-yl group, and the combination of R¹, R⁴, R⁵, R⁸, and R⁹ represents any one of the combinations shown in [Table L1] to [Table L4](hereinafter, referred to as a compound group SX162).

A compound according to the compound (L-1), wherein Y represents a single bond, p represents 0, Q represents a CH₂-7-methoxybenzofuran-2-yl group, and the combination of R¹, R⁴, R⁵, R⁸, and R⁹ represents any one of the combinations shown in [Table L1] to [Table L4](hereinafter, referred to as a compound group SX163).

A compound according to the compound (L-1), wherein Y represents a single bond, p represents 0, Q represents a CH₂-4,5-dimethylbenzofuran-2-yl group, and the combination of R¹, R⁴, R⁵, R⁸, and R⁹ represents any one of the combinations shown in [Table L1] to [Table L4](hereinafter, referred to as a compound group SX164).

A compound according to the compound (L-1), wherein Y represents a single bond, p represents 0, Q represents a CH₂-4,6-dimethylbenzofuran-2-yl group, and the combination of R¹, R⁴, R⁵, R⁸, and R⁹ represents any one of the combinations shown in [Table L1] to [Table L4](hereinafter, referred to as a compound group SX165).

A compound according to the compound (L-1), wherein Y represents a single bond, p represents 0, Q represents a CH₂-4,7-dimethylbenzofuran-2-yl group, and the combination of R¹, R⁴, R⁵, R⁸, and R⁹ represents any one of the combinations shown in [Table L1] to [Table L4](hereinafter, referred to as a compound group SX166).

A compound according to the compound (L-1), wherein Y represents a single bond, p represents 0, Q represents a CH₂-5,6-dimethylbenzofuran-2-yl group, and the combination of R¹, R⁴, R⁵, R⁸, and R⁹ represents any one of the combinations shown in [Table L1] to [Table L4](hereinafter, referred to as a compound group SX167).

A compound according to the compound (L-1), wherein Y represents a single bond, p represents 0, Q represents a CH₂-5,7-dimethylbenzofuran-2-yl group, and the combination of R¹, R⁴, R⁵, R⁸, and R⁹ represents any one of the combinations shown in [Table L1] to [Table L4](hereinafter, referred to as a compound group SX168).

A compound according to the compound (L-1), wherein Y represents a single bond, p represents 0, Q represents a CH₂-6,7-dimethylbenzofuran-2-yl group, and the combination of R¹, R⁴, R⁵, R⁸, and R⁹ represents any one of the combinations shown in [Table L1] to [Table L4](hereinafter, referred to as a compound group SX169).

A compound according to the compound (L-1), wherein Y represents a single bond, p represents 0, Q represents a CH₂-benzothiophene-2-yl group, and the combination of R¹, R⁴, R⁵, R⁸, and R⁹ represents any one of the combinations shown in [Table L1] to [Table L4](hereinafter, referred to as a compound group SX170).

A compound according to the compound (L-1), wherein Y represents a single bond, p represents 0, Q represents a CH₂-3-methylbenzothiophene-2-yl group, and the combination of R¹, R⁴, R⁵, R⁸, and R⁹ represents any one of the combinations shown in [Table L1] to [Table L4](hereinafter, referred to as a compound group SX171).

A compound according to the compound (L-1), wherein Y represents a single bond, p represents 0, Q represents a CH₂-4-methylbenzothiophene-2-yl group, and the combination of R¹, R⁴, R⁵, R⁸, and R⁹ represents any one of the combinations shown in [Table L1] to [Table L4](hereinafter, referred to as a compound group SX172).

A compound according to the compound (L-1), wherein Y represents a single bond, p represents 0, Q represents a CH₂-5-methylbenzothiophene-2-yl group, and the combination of R¹, R⁴, R⁵, R⁸, and R⁹ represents any one of the combinations shown in [Table L1] to [Table L4](hereinafter, referred to as a compound group SX173).

A compound according to the compound (L-1), wherein Y represents a single bond, p represents 0, Q represents a CH₂-6-methylbenzothiophene-2-yl group, and the combination of R¹, R⁴, R⁵, R⁸, and R⁹ represents any one of the combinations shown in [Table L1] to [Table L4](hereinafter, referred to as a compound group SX174).

A compound according to the compound (L-1), wherein Y represents a single bond, p represents 0, Q represents a CH₂-7-methylbenzothiophene-2-yl group, and the combination of R¹, R⁴, R⁵, R⁸, and R⁹ represents any one of the combinations shown in [Table L1] to [Table L4](hereinafter, referred to as a compound group SX175).

A compound according to the compound (L-1), wherein Y represents a single bond, p represents 0, Q represents a CH₂-3-fluorobenzothiophene-2-yl group, and the combination of R¹, R⁴, R⁵, R⁸, and R⁹ represents any one of the combinations shown in [Table L1] to [Table L4](hereinafter, referred to as a compound group SX176).

A compound according to the compound (L-1), wherein Y represents a single bond, p represents 0, Q represents a CH₂-4-fluorobenzothiophene-2-yl group, and the combination of R¹, R⁴, R⁵, R⁸, and R⁹ represents any one of the combinations shown in [Table L1] to [Table L4](hereinafter, referred to as a compound group SX177).

A compound according to the compound (L-1), wherein Y represents a single bond, p represents 0, Q represents a CH₂-5-fluorobenzothiophene-2-yl group, and the combination of R¹, R⁴, R⁵, R⁸, and R⁹ represents any one of the combinations shown in [Table L1] to [Table L4](hereinafter, referred to as a compound group SX178).

A compound according to the compound (L-1), wherein Y represents a single bond, p represents 0, Q represents a CH₂-6-fluorobenzothiophene-2-yl group, and the combination of R¹, R⁴, R⁵, R⁸, and R⁹ represents any one of the combinations shown in [Table L1] to [Table L4](hereinafter, referred to as a compound group SX179).

A compound according to the compound (L-1), wherein Y represents a single bond, p represents 0, Q represents a CH₂-7-fluorobenzothiophene-2-yl group, and the combination of R¹, R⁴, R⁵, R⁸, and R⁹ represents any one of the combinations shown in [Table L1] to [Table L4](hereinafter, referred to as a compound group SX180).

A compound according to the compound (L-1), wherein Y represents a single bond, p represents 0, Q represents a CH₂-3-chlorobenzothiophene-2-yl group, and the combination of R¹, R⁴, R⁵, R⁸, and R⁹ represents any one of the combinations shown in [Table L1] to [Table L4](hereinafter, referred to as a compound group SX181).

A compound according to the compound (L-1), wherein Y represents a single bond, p represents 0, Q represents a CH₂-4-chlorobenzothiophene-2-yl group, and the combination of R¹, R⁴, R⁵, R⁸, and R⁹ represents any one of the combinations shown in [Table L1] to [Table L4](hereinafter, referred to as a compound group SX182).

A compound according to the compound (L-1), wherein Y represents a single bond, p represents 0, Q represents a CH₂-5-chlorobenzothiophene-2-yl group, and the combination of R¹, R⁴, R⁵, R⁸, and R⁹ represents any one of the combinations shown in [Table L1] to [Table L4](hereinafter, referred to as a compound group SX183).

A compound according to the compound (L-1), wherein Y represents a single bond, p represents 0, Q represents a CH₂-6-chlorobenzothiophene-2-yl group, and the combination of R¹, R⁴, R⁵, R⁸, and R⁹ represents any one of the combinations shown in [Table L1] to [Table L4](hereinafter, referred to as a compound group SX184).

A compound according to the compound (L-1), wherein Y represents a single bond, p represents 0, Q represents a CH₂-7-chlorobenzothiophene-2-yl group, and the combination of R¹, R⁴, R⁵, R⁸, and R⁹ represents any one of the combinations shown in [Table L1] to [Table L4](hereinafter, referred to as a compound group SX185).

A compound according to the compound (L-1), wherein Y represents a single bond, p represents 0, Q represents a CH₂-3-methoxybenzothiophene-2-yl group, and the combination of R¹, R⁴, R⁵, R⁸, and R⁹ represents any one of the combinations shown in [Table L1] to [Table L4](hereinafter, referred to as a compound group SX186).

A compound according to the compound (L-1), wherein Y represents a single bond, p represents 0, Q represents a CH₂-4-methoxybenzothiophene-2-yl group, and the combination of R¹, R⁴, R⁵, R⁸, and R⁹ represents any one of the combinations shown in [Table L1] to [Table L4](hereinafter, referred to as a compound group SX187).

A compound according to the compound (L-1), wherein Y represents a single bond, p represents 0, Q represents a CH₂-5-methoxybenzothiophene-2-yl group, and the combination of R¹, R⁴, R⁵, R⁸, and R⁹ represents any one of the combinations shown in [Table L1] to [Table L4](hereinafter, referred to as a compound group SX188).

A compound according to the compound (L-1), wherein Y represents a single bond, p represents 0, Q represents a CH₂-6-methoxybenzothiophene-2-yl group, and the combination of R¹, R⁴, R⁵, R⁸, and R⁹ represents any one of the combinations shown in [Table L1] to [Table L4](hereinafter, referred to as a compound group SX189).

A compound according to the compound (L-1), wherein Y represents a single bond, p represents 0, Q represents a CH₂-7-methoxybenzothiophene-2-yl group, and the combination of R¹, R⁴, R⁵, R⁸, and R⁹ represents any one of the combinations shown in [Table L1] to [Table L4](hereinafter, referred to as a compound group SX190).

A compound according to the compound (L-1), wherein Y represents a single bond, p represents 0, Q represents a CH₂-4,5-dimethylbenzothiophene-2-yl group, and the combination of R¹, R⁴, R⁵, R⁸, and R⁹ represents any one of the combinations shown in [Table L1] to [Table L4](hereinafter, referred to as a compound group SX191).

A compound according to the compound (L-1), wherein Y represents a single bond, p represents 0, Q represents a CH₂-4,6-dimethylbenzothiophene-2-yl group, and the combination of R¹, R⁴, R⁵, R⁸, and R⁹ represents any one of the combinations shown in [Table L1] to [Table L4](hereinafter, referred to as a compound group SX192).

A compound according to the compound (L-1), wherein Y represents a single bond, p represents 0, Q represents a CH₂-4,7-dimethylbenzothiophene-2-yl group, and the combination of R¹, R⁴, R⁵, R⁸, and R⁹ represents any one of the combinations shown in [Table L1] to [Table L4](hereinafter, referred to as a compound group SX193).

A compound according to the compound (L-1), wherein Y represents a single bond, p represents 0, Q represents a CH₂-5,6-dimethylbenzothiophene-2-yl group, and the combination of R¹, R⁴, R⁵, R⁸, and R⁹ represents any one of the combinations shown in [Table L1] to [Table L4](hereinafter, referred to as a compound group SX194).

A compound according to the compound (L-1), wherein Y represents a single bond, p represents 0, Q represents a CH₂-5,7-dimethylbenzothiophene-2-yl group, and the combination of R¹, R⁴, R⁵, R⁸, and R⁹ represents any one of the combinations shown in [Table L1] to [Table L4](hereinafter, referred to as a compound group SX195).

A compound according to the compound (L-1), wherein Y represents a single bond, p represents 0, Q represents a CH₂-6,7-dimethylbenzothiophene-2-yl group, and the combination of R¹, R⁴, R⁵, R³, and R⁹ represents any one of the combinations shown in [Table L1] to [Table L4](hereinafter, referred to as a compound group SX196).

A compound according to the compound (L-1), wherein Y represents a single bond, p represents 0, Q represents a (CH₂)₃ phenyl group, and the combination of R¹, R⁴, R⁵, R³, and R⁹ represents any one of the combinations shown in [Table L1] to [Table L4](hereinafter, referred to as a compound group SX197).

A compound according to the compound (L-1), wherein Y represents a single bond, p represents 0, Q represents a (CH₂)₃-2-fluorophenyl group, and the combination of R¹, R⁴, R⁵, R³, and R⁹ represents any one of the combinations shown in [Table L1] to [Table L4](hereinafter, referred to as a compound group SX198).

A compound according to the compound (L-1), wherein Y represents a single bond, p represents 0, Q represents a (CH₂)₃-3-fluorophenyl group, and the combination of R¹, R⁴, R⁵, R³, and R⁹ represents any one of the combinations shown in [Table L1] to [Table L4](hereinafter, referred to as a compound group SX199).

A compound according to the compound (L-1), wherein Y represents a single bond, p represents 0, Q represents a (CH₂)₃-4-fluorophenyl group, and the combination of R¹, R⁴, R⁵, R⁸, and R⁹ represents any one of the combinations shown in [Table L1] to [Table L4](hereinafter, referred to as a compound group SX200).

A compound according to the compound (L-1), wherein Y represents a single bond, p represents 0, Q represents a (CH₂)₃-2-chlorophenyl group, and the combination of R¹, R⁴, R⁵, R³, and R⁹ represents any one of the combinations shown in [Table L1] to [Table L4](hereinafter, referred to as a compound group SX201).

A compound according to the compound (L-1), wherein Y represents a single bond, p represents 0, Q represents a (CH₂)₃-3-chlorophenyl group, and the combination of R¹, R⁴, R⁵, R⁸, and R⁹ represents any one of the combinations shown in [Table L1] to [Table L4](hereinafter, referred to as a compound group SX202).

A compound according to the compound (L-1), wherein Y represents a single bond, p represents 0, Q represents a (CH₂)₃-4-chlorophenyl group, and the combination of R¹, R⁴, R⁵, R⁸, and R⁹ represents any one of the combinations shown in [Table L1] to [Table L4](hereinafter, referred to as a compound group SX203).

A compound according to the compound (L-1), wherein Y represents a single bond, p represents 0, Q represents a (CH₂)₃-2-methylphenyl group, and the combination of R¹, R⁴, R⁵, R³, and R⁹ represents any one of the combinations shown in [Table L1] to [Table L4](hereinafter, referred to as a compound group SX204).

A compound according to the compound (L-1), wherein Y represents a single bond, p represents 0, Q represents a (CH₂)₃-3-methylphenyl group, and the combination of R¹, R⁴, R⁵, R⁸, and R⁹ represents any one of the combinations shown in [Table L1] to [Table L4](hereinafter, referred to as a compound group SX205).

A compound according to the compound (L-1), wherein Y represents a single bond, p represents 0, Q represents a (CH₂)₃-4-methylphenyl group, and the combination of R¹, R⁴, R⁵, R³, and R⁹ represents any one of the combinations shown in [Table L1] to [Table L4](hereinafter, referred to as a compound group SX206).

A compound according to the compound (L-1), wherein Y represents a single bond, p represents 0, Q represents a (CH₂)₃-2-methoxyphenyl group, and the combination of R¹, R⁴, R⁵, R⁸, and R⁹ represents any one of the combinations shown in [Table L1] to [Table L4](hereinafter, referred to as a compound group SX207).

A compound according to the compound (L-1), wherein Y represents a single bond, p represents 0, Q represents a (CH₂)₃-3-methoxyphenyl group, and the combination of R¹, R⁴, R⁵, R³, and R⁹ represents any one of the combinations shown in [Table L1] to [Table L4](hereinafter, referred to as a compound group SX208).

A compound according to the compound (L-1), wherein Y represents a single bond, p represents 0, Q represents a (CH₂)₃-4-methoxyphenyl group, and the combination of R¹, R⁴, R⁵, R⁸, and R⁹ represents any one of the combinations shown in [Table L1] to [Table L4](hereinafter, referred to as a compound group SX209).

A compound according to the compound (L-1), wherein Y represents a single bond, p represents 0, Q represents a (CH₂)₃-2-naphthyl group, and the combination of R¹, R⁴, R⁵, R³, and R⁹ represents any one of the combinations shown in [Table L1] to [Table L4](hereinafter, referred to as a compound group SX210).

Then, formulation examples of the Compound of the present invention will be described. Parts represent parts by weight. In addition, the Compound S of the present invention represents a compound described in compound groups SX1 to SX210 or any Compound of the present invention compounds 1 to 108.

### Formulation Example 1

35 parts of a mixture of polyoxyethylene alkyl ether sulfate ammonium salt and silica (weight ratio 1:1), 10 parts of any one of the Compound S of the present invention, and 55 parts of water are mixed, and finely pulverized by wet grinding method to provide a formulation.

### Formulation Example 2

50 parts of any one of the Compound S of the present invention, 3 parts of calcium lignosulfonate, 2 parts of sodium lauryl sulfate, and 45 parts of silica are pulverized and mixed to provide a formulation.

### Formulation Example 3

5 parts of any one of the Compound S of the present invention, 9 parts of polyoxyethylene styrylphenyl ether, 5 parts of polyoxyethylene decyl ether (addition number of ethylene oxide: 5), 6 parts of calcium dodecylbenzene sulfonate, and 75 parts of xylene are mixed to provide a formulation.

### Formulation Example 4

2 parts of any one of the Compound S of the present invention, 1 part of silica, 2 parts of calcium lignosulfonate, 30 parts of bentonite, and 65 parts of kaolin clay are pulverized and mixed, an appropriate amount of water is added thereto, the mixture is kneaded, granulated by a granulator, and then dried to provide a formulation.

### Formulation Example 5

10 parts of any one of the Compound S of the present invention is mixed with a mixture of 18 parts of benzyl alcohol and 9 parts of DMSO, 6.3 parts of GERONOL (registered trademark) TE250, 2.7 parts of Ethylan (registered trademark) NS-500LQ, and 54 parts of solvent naphtha are added thereto, and the mixture is mixed to provide a formulation.

### Formulation Example 6

0.1 parts of any one of the Compound S of the present invention and 39.9 parts of kerosene are mixed and dissolved, and placed in an aerosol container, and filled with 60 parts of liquefied petroleum gas (mixture of propane, butane, and isobutane and saturated vapor pressure: 0.47 MPa (25° C.)) to provide a formulation.

### Formulation Example 7

0.2 parts of any one of the Compound S of the present invention, 50 parts of pyrethrum extract cake powder, 30 parts of tab powder, and 19.8 parts of wood powder are mixed, an appropriate amount of water is added thereto, and the mixture is kneaded, then subjected to an extruder to form a plate-shaped sheet, and formed into a spiral shape by a punching machine, thereby providing a formulation.

Then, the efficacy of the Compound of the present invention against harmful arthropod is shown by Test Examples. In the following Test Examples, the test was performed at 25° C.

### Test Method 1

A test compound is formed into a formulation according to the method described in Formulation Example 1, and water containing 0.03% by volume of Sindain (registered trademark) is added thereto to prepare a dilution containing the test compound at a predetermined concentration.

A cucumber (Cucumis sativus) seedling (second true leaf stage) planted in a container is inoculated with about 30 cotton aphids (Aphis gossypii) (whole stage). One day later, the dilution is sprayed onto the seedlings at a rate of 10 mL/seedling. After 5 days, the number of surviving insects is examined, and the controlling value is determined by the following formula.

$$Controlling value ( \% ) = \{ 1 - ( Cb \times Tai ) / ( Cai \times Tb ) \} \times 100$$

wherein the symbols in the formula represent the following meanings.
Cb: number of test insects in a non-treated section
Cai: number of surviving insects in a non-treated section at the time of investigation
Tb: number of test insects in a treated section
Tai: number of surviving insects in a treated section at the time of investigation

Herein, the non-treated section means a section that is subjected to the same operation as that of the treated section except that the test compound is not used.

### Test Example 1-1

As a result of performing a test according to test method 1 using the following Compound of the present invention as a test compound at a predetermined concentration of 500 ppm, the following Compounds of the present invention all exhibited a controlling value of 90% or more.

### Compound of the Present Invention: 29, 91

### Test Method 2

A test compound is formed into a formulation according to the method described in Formulation Example 1, and water containing 0.03% by volume of Sindain (registered trademark) is added thereto to prepare a dilution containing the test compound at a predetermined concentration.

A cabbage (Brassicae oleracea) seedling (2 to 3 true leaf stage) planted in a container is sprayed with the dilution at a rate of 20 mL/seedling. Thereafter, the foliage portion of the seedling is cut out and placed in a container on which filter paper is laid. Thereto are released five second instar larvae of common cutworm (Spodoptera litura). After 5 days, the number of surviving insects is counted, and the mortality is determined from the following equation.

$$Mortality ( \% ) = ( 1 - number of surviving insects / 5 ) \times 100$$

### Test Example 2-1

As a result of performing a test according to test method 2 using the following Compound of the present invention as a test compound at a predetermined concentration of 500 ppm, the following Compounds of the present invention all exhibited a mortality of 80% or more.

Compound of the present invention: 14, 16, 18, 20, 21, 22, 24, 25, 26, 28, 29, 30, 31, 32, 34, 36, 37, 38, 39, 41, 43, 44, 45, 46, 47, 49, 50, 51, 52, 53, 54, 55, 58, 59, 60, 61, 62, 63, 67, 68, 69, 71, 73, 75, 78, 79, 82, 85, 86, 89, 91, 92, 93, 95, 99, 100, 103, 104

### Test Method 3

A test compound is formed into a formulation according to the method described in Formulation Example 1, and water containing 0.03% by volume of Sindain (registered trademark) is added thereto to prepare a dilution containing the test compound at a predetermined concentration.

A cabbage (Brassicae oleracea) seedling (2 to 3 true leaf stage) planted in a container is sprayed with the dilution at a rate of 20 mL/seedling. Thereafter, the foliage portion of the seedling is cut out and placed in a container on which filter paper is laid. Thereto are released five second instar larvae of diamondback moth (Plutella xylostella). After 5 days, the number of surviving insects is counted, and the mortality is determined from the following equation.

$$Mortality ( \% ) = ( 1 - number of surviving insects / 5 ) \times 100$$

### Test Example 3-1

As a result of performing a test according to test method 3 using the following Compound of the present invention as a test compound at a predetermined concentration of 500 ppm, the following Compounds of the present invention all exhibited a mortality of 80% or more.

Compound of the present invention: 6, 14, 16, 17, 18, 20, 21, 22, 23, 24, 25, 26, 27, 28, 29, 32, 34, 35, 36, 37, 38, 39, 41, 46, 47, 48, 49, 50, 51, 52, 55, 57, 58, 59, 60, 61, 62, 63, 65, 66, 67, 68, 69, 73, 74, 75, 77, 78, 79, 80, 82, 83, 84, 85, 86, 89, 90, 91, 92, 93, 95, 96, 97, 99, 100, 101, 102, 103, 104, 105, 106

### Test Method 4

Each 1 mg of the test compound is dissolved in 50 μL of a mixed solution of polyoxyethylene sorbitan monococoate and acetone (5:95 (volume ratio)). Water containing 0.03% by volume of Sindain (registered trademark) is added thereto to prepare a dilution containing the test compound at a predetermined concentration.

Young seedlings of maize (Zea mays) are immersed in the dilution for 30 seconds. Thereafter, the two fruits are placed in a petri dish (90 mm diameter), and thereto are released 10 second instar larvae of Western corn rootworm (Diabrotica virgifera virgifera). After 5 days, the number of dead insects is counted, and the mortality is determined from the following equation.

$$Mortality ( \% ) = ( number of dead insects / 10 ) \times 100$$

### Test Example 4-1

The control effect can be confirmed by performing a test in accordance with Test method 4 using the Compound of the present invention as a test compound at a predetermined concentration of 200 ppm.

### Compound of the Present Invention:

### Test Method 5

An acetone solution prepared such that the amount of the test compound is 800 ppm is poured into a container having an internal volume of 50 mL, and the inner surface of the container is uniformly coated such that the amount of the test compound is 40 mg/m², and then dried.

Five adult male German cockroaches (Blattella germanica) are placed in the container, and the lid is closed. After a lapse of a predetermined time, the state of German cockroaches (Blattella germanica) is examined, and the mortality is determined. The mortality is calculated by the following equation.

$$Mortality ( \% ) = ( number of dead insects / number of tested insects ) \times 100$$

### Test Example 5-1

As a result of performing a test according to test method 5 using the following Compound of the present invention as a test compound at a predetermined time of one hour, the following Compounds of the present invention all exhibited a mortality of 80% or more.

Compound of the present invention: 4, 5, 6, 7, 8, 9, 10, 11, 12, 15, 49, 50, 51, 56, 58, 65, 93

### Test Example 5-2

As a result of performing a test according to test method 5 using the following Compound of the present invention as a test compound at a predetermined time of one day, the following Compounds of the present invention all exhibited a mortality of 80% or more.

Compound of the present invention: 4, 5, 6, 7, 8, 9, 11, 12, 15, 16, 18, 19, 20, 21, 23, 24, 25, 26, 28, 29, 30, 31, 32, 33, 34, 35, 36, 37, 38, 39, 40, 41, 42, 43, 45, 46, 47, 48, 49, 50, 51, 52, 56, 57, 58, 60, 62, 63, 65, 66, 67, 68, 69, 70, 71, 72, 73, 74, 75, 76, 77, 78, 79, 82, 83, 85, 86, 89, 92, 93, 95, 96, 99, 104, 105, 106, 107, 108

### Test Example 5-3

As a result of performing a test according to test method 5 using the following Compound of the present invention as a test compound at a predetermined time of three days, the following Compounds of the present invention all exhibited a mortality of 80% or more.

Compound of the present invention: 3, 4, 5, 6, 7, 8, 9, 11, 12, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 55, 56, 57, 58, 59, 60, 61, 62, 63, 65, 66, 67, 68, 69, 70, 71, 72, 73, 74, 75, 76, 77, 78, 79, 82, 83, 85, 86, 89, 91, 92, 93, 95, 96, 97, 98, 99, 100, 104, 105, 106, 107, 108

### Test Method 6

An acetone solution prepared such that the amount of the test compound is 2000 ppm is poured into a container having an internal volume of 20 mL, and the inner surface of the container is uniformly coated such that the amount of the test compound is 100 mg/m², and then dried.

Five nymphs of Haemaphysalis longicornis are placed in the container, and the lid is closed. After a lapse of a predetermined time, the state of Haemaphysalis longicornis is examined to determine the mortality. The mortality is calculated by the following equation.

$$Mortality ( \% ) = ( number of dead insects / number of tested insects ) \times 100$$

### Test Example 6-1

As a result of performing a test according to test method 6 using the following Compound of the present invention as a test compound at a predetermined time of two days, the following Compounds of the present invention all exhibited a mortality of 80% or more.

Compound of the present invention: 1, 2, 3, 5, 6, 16, 18, 20, 21, 23, 25, 26, 27, 28, 29, 31, 32, 33, 34, 35, 36, 37, 38, 39, 40, 42, 45, 46, 47, 49, 50, 51, 56, 57, 58, 59, 60, 61, 62

### Test Method 7

An acetone solution prepared such that the amount of the test compound is 800 ppm is poured into a container having an internal volume of 20 mL, and the inner surface of the container is uniformly coated such that the amount of the test compound is 40 mg/m², and then dried.

Five adult female house flies (Musca domestica) are placed in the container, and the lid is closed. After a lapse of a predetermined time, the state of house flies (Musca domestica) is examined, and the mortality is determined. The mortality is calculated by the following equation.

$$Mortality ( \% ) = ( number of dead insects / number of tested insects ) \times 100$$

### Test Example 7-1

As a result of performing a test according to test method 7 using the following Compound of the present invention as a test compound at a predetermined time of one hour, the following Compounds of the present invention all exhibited a mortality of 80% or more.

Compound of the present invention: 3, 4, 5, 6, 7, 8, 9, 10, 12, 15, 18, 19, 20, 23, 24, 25, 26, 28, 30, 31, 32, 33, 34, 35, 36, 37, 38, 39, 40, 41, 42, 43, 45, 46, 47, 48, 49, 50, 51, 56, 57, 58, 60, 62, 63, 65, 66, 67, 68, 69, 70, 71, 72, 73, 74, 75, 76, 77, 78, 79, 82, 85, 89, 92, 93, 98, 99, 100, 105, 107

### Test Example 7-2

As a result of performing a test according to test method 7 using the following Compound of the present invention as a test compound at a predetermined time of one day, the following Compounds of the present invention all exhibited a mortality of 80% or more.

Compound of the present invention: 3, 4, 5, 6, 7, 8, 10, 13, 14, 15, 16, 18, 19, 20, 21, 22, 23, 24, 25, 26, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 65, 66, 67, 68, 69, 70, 71, 72, 73, 74, 75, 76, 77, 78, 79, 82, 83, 85, 86, 89, 92, 93, 96, 98, 99, 100, 103, 104, 105, 106, 107, 108

### Test Method 8

An acetone solution prepared such that the amount of the test compound is 200 ppm is poured into a container having an internal volume of 20 mL, and the inner surface of the container is uniformly coated such that the amount of the test compound is 10 mg/m², and then dried. Five adult female common house mosquitos (Culex pipiens pallens) are placed in the container, and the lid is closed. After a lapse of a predetermined time, the state of common house mosquitos (Culex pipiens pallens) is examined, and the mortality is determined. The mortality is calculated by the following equation.

$$Mortality ( \% ) = ( number of dead insects / number of tested insects ) \times 100$$

### Test Example 8-1

As a result of performing a test according to test method 8 using the following Compound of the present invention as a test compound at a predetermined time of one hour, the following Compounds of the present invention all exhibited a mortality of 80% or more.

Compound of the present invention: 3, 4, 5, 6, 7, 8, 9, 10, 11, 12, 15, 19, 25, 26, 28, 30, 31, 34, 35, 36, 38, 39, 40, 41, 42, 44, 47, 48, 49, 50, 51, 56, 57, 58, 60, 63, 64, 65, 66, 67, 68, 69, 70, 71, 75, 76, 77, 78, 79, 82, 83, 86, 92, 93, 100, 106, 107

### Test Example 8-2

As a result of performing a test according to test method 8 using the following Compound of the present invention as a test compound at a predetermined time of one day, the following Compounds of the present invention all exhibited a mortality of 80% or more.

Compound of the present invention: 1, 2, 3, 4, 5, 6, 7, 8, 9, 10, 11, 12, 13, 15, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 82, 83, 84, 85, 86, 89, 91, 92, 93, 96, 97, 98, 99, 100, 103, 104, 105, 106, 107, 108

### Test Method 9

An acetone solution prepared such that the amount of the test compound is 50 ppm is poured into a container having an internal volume of 20 mL, and the inner surface of the container is uniformly coated such that the amount of the test compound is 2.5 mg/m², and then dried. 10 adult female Pharaoh ants (Monomorium pharaonis) are placed in the container, and the lid is closed. After a lapse of a predetermined time, the state of Pharaoh ants (Monomorium pharaonis) is examined, and the mortality is determined. The mortality is calculated by the following equation.

$$Mortality ( \% ) = ( number of dead insects / number of tested insects ) \times 100$$

### Test Example 9-1

As a result of performing a test according to test method 9 using the following Compound of the present invention as a test compound at a predetermined time of one hour, the following Compounds of the present invention all exhibited a mortality of 70% or more.

Compound of the present invention: 4, 5, 6, 8, 11, 12, 15, 18, 19, 20, 21, 23, 24, 25, 26, 27, 28, 29, 30, 31, 32, 33, 34, 35, 36, 38, 39, 40, 41, 42, 43, 44, 45, 46, 47, 49, 51, 53, 58, 60, 63, 65, 66, 68, 69, 70, 71, 73, 75, 78, 79, 82, 85, 86, 89, 91, 92, 93, 95, 96, 97, 98, 99, 100, 103, 104, 105, 106, 107

### Test Example 9-2

As a result of performing a test according to test method 9 using the following Compound of the present invention as a test compound at a predetermined time of one day, the following Compounds of the present invention all exhibited a mortality of 70% or more.

Compound of the present invention: 4, 5, 6, 7, 8, 9, 11, 12, 15, 16, 17, 18, 19, 20, 21, 22, 23, 24, 25, 26, 27, 28, 29, 30, 31, 32, 33, 34, 35, 36, 38, 39, 40, 41, 42, 43, 44, 45, 46, 47, 48, 49, 50, 51, 52, 53, 54, 55, 56, 57, 58, 59, 60, 61, 62, 63, 65, 66, 67, 68, 69, 70, 71, 72, 73, 74, 75, 78, 79, 82, 83, 85, 86, 89, 90, 91, 92, 93, 95, 96, 97, 98, 99, 100101, 103, 104, 105, 106, 107, 108

### Test Method 10

A test compound is formed into a formulation according to the method described in Formulation Example 5, and water containing 0.03% by volume of Sindain (registered trademark) is added thereto to prepare a dilution containing the test compound at a predetermined concentration.

A cucumber (Cucumis sativus) seedling (second true leaf stage) planted in a container is sprayed with the dilution at a rate of 10 mL/seedling. Thereafter, the first true leaf is cut out and stored in a container, and approximately 20 larvae of western flower thrips (Frankliniella occidentalis) are released into the container. After 7 days, the number of surviving insects is examined, and the mortality is determined by the following equation.

$$Mortality ( \% ) = \{ 1 - number of surviving insects / 20 \} \times 100$$

### Test Example 10-1

As a result of performing a test according to test method 10 using the following Compound of the present invention as a test compound at a predetermined concentration of 200 ppm, the following Compounds of the present invention all exhibited a mortality of 90% or more.

Compound of the present invention: 18, 20, 21, 22, 25, 29, 30, 31, 37, 38, 39, 43, 45, 47, 57, 58, 60, 62, 63, 65, 66, 67, 68, 69, 70, 72, 78, 79, 82, 89, 93, 97, 99, 106

### Test Method 11

A test compound is formed into a formulation according to the method described in Formulation Example 5, and water containing 0.03% by volume of Sindain (registered trademark) is added thereto to prepare a dilution containing the test compound at a predetermined concentration.

A cabbage (Brassicae oleracea) seedling (3 to 4 true leaf stage) planted in a container is sprayed with the dilution at a rate of 20 mL/seedling. Thereafter, 10 third instar larvae of common cutworm (Spodoptera litura) are released. After 6 days, the number of surviving insects is counted, and the mortality is determined from the following equation.

$$Mortality ( \% ) = ( 1 - number of surviving insects / 10 ) \times 100$$

### Test Example 11-1

As a result of performing a test according to test method 11 using the following Compound of the present invention as a test compound at a predetermined concentration of 200 ppm, the following Compounds of the present invention all exhibited a mortality of 80% or more.

Compound of the present invention: 14, 16, 18, 20, 21, 22, 25, 28, 29, 30, 31, 32, 34, 36, 37, 38, 39, 41, 43, 45, 46, 47, 49, 50, 51, 55, 58, 60, 61, 62, 67, 68, 69, 71, 73, 75, 79, 85, 89, 91, 92, 93, 95, 99, 100, 103

### Test Method 12

A test compound is formed into a formulation according to the method described in Formulation Example 5, and water containing 0.03% by volume of Sindain (registered trademark) is added thereto to prepare a dilution containing the test compound at a predetermined concentration.

A cabbage (Brassicae oleracea) seedling (3 to 4 true leaf stage) planted in a container is sprayed with the dilution at a rate of 20 mL/seedling. Thereafter, 10 third instar larvae of diamondback moth (Plutella xylostella) are released. After 5 days, the number of surviving insects is counted, and the mortality is determined from the following equation.

$$Mortality ( \% ) = ( 1 - number of surviving insects / 10 ) \times 100$$

### Test Example 12-1

As a result of performing a test according to test method 12 using the following Compound of the present invention as a test compound at a predetermined concentration of 200 ppm, the following Compounds of the present invention all exhibited a mortality of 80% or more.

Compound of the present invention: 14, 16, 17, 18, 20, 21, 22, 23, 28, 29, 32, 34, 35, 36, 37, 38, 39, 41, 46, 47, 49, 50, 51, 55, 60, 61, 62, 63, 65, 67, 68, 69, 73, 74, 75, 78, 79, 82, 83, 84, 90, 95, 96, 97, 99, 100, 103, 105, 106

### Test Method 13

A test compound is formed into a formulation according to the method described in Formulation Example 5, and water containing 0.03% by volume of Sindain (registered trademark) is added thereto to prepare a dilution containing the test compound at a predetermined concentration.

A cucumber (Cucumis sativus) seedling (second true leaf stage) planted in a container is inoculated with about 30 cotton aphids (Aphis gossypii) (whole stage). One day later, the dilution is sprayed onto the seedlings at a rate of 10 mL/seedling. After 5 days, the number of surviving insects is examined, and the controlling value is determined by the following formula.

$$Controlling value ( \% ) = \{ 1 - ( Cb \times Tai ) / ( Cai \times Tb ) \} \times 100$$

wherein the symbols in the formula represent the following meanings.
Cb: number of test insects in a non-treated section
Cai: number of surviving insects in a non-treated section at the time of investigation
Tb: number of test insects in a treated section
Tai: number of surviving insects in a treated section at the time of investigation

Herein, the non-treated section means a section that is subjected to the same operation as that of the treated section except that the test compound is not used.

### Test Example 13-1

The control effect can be confirmed by performing a test in accordance with Test method 13 using the Compound of the present invention as a test compound at a predetermined concentration of 200 ppm.

### Compound of the Present Invention:

### Test Method 14

The test compound is formulated according to the method described in Formulation Example 1, and water is added thereto to prepare a dilution containing the test compound at a predetermined concentration.

30 last-instar larvae of common house mosquito (Culex pipiens pallens) are released into the dilution, and the state of the larvae of common house mosquito (Culex pipiens) is examined one day later to determine the mortality. The mortality is calculated by the following equation.

$$Mortality ( \% ) = ( number of dead insects / number of tested insects ) \times 100$$

### Test Example 14-1

As a result of performing a test according to test method 14 using the following Compound of the present invention as a test compound at a predetermined concentration of 3.5 ppm, the following Compounds of the present invention all exhibited a mortality of 91% or more.

Compound of the present invention: 11, 14, 16, 18, 20, 21, 22, 24, 25, 26, 27, 28, 29, 30, 31, 32, 33, 34, 35, 36, 37, 38, 39, 40, 41, 42, 43, 45, 46, 47, 48, 49, 50, 51, 53, 55, 58, 59, 60, 61, 62, 65, 66, 67, 68, 69, 70, 71, 72, 73, 75, 77, 78, 79, 81, 82, 8385, 89, 93, 97, 102, 104

### Test Method 15

A test compound is formed into a formulation according to the method described in Formulation Example 1, and water containing 0.03% by volume of Sindain (registered trademark) is added thereto to prepare a dilution containing the test compound at a predetermined concentration.

A rice (Oryza sativa) seedling (second leaf stage) planted in a container is sprayed with the dilution at a rate of 10 mL/seedling. Thereafter, 20 third-instar nymphs of brown planthopper (Nilaparvata lugens) are released. After 6 days, the number of surviving insects is examined, and the mortality is determined by the following equation.

$$Mortality ( \% ) = \{ 1 - number of surviving insects / 20 \} \times 100$$

### Test Example 15-1

As a result of performing a test according to Test method 15 using the following Compound of the present invention as a test compound at a predetermined concentration of 500 ppm, the following Compounds of the present invention all exhibited a mortality of 90% or more.

Compound of the present invention: 18, 20, 25, 26, 29, 34, 35, 36, 38, 42, 45, 46, 48, 50, 51, 52, 57, 58, 60, 62, 63, 65, 66, 67, 68, 69, 70, 71, 72, 73, 74, 75, 76, 77, 78, 79, 82, 83, 85, 106

### Test Method 16

A test compound is formed into a formulation according to the method described in Formulation Example 5, and water containing 0.03% by volume of Sindain (registered trademark) is added thereto to prepare a dilution containing the test compound at a predetermined concentration.

A rice (Oryza sativa) seedling (second leaf stage) planted in a container is sprayed with the dilution at a rate of 10 mL/seedling. Thereafter, 20 third-instar nymphs of the brown planthopper (Nilaparvata lugens) are released. After 6 days, the number of surviving insects is examined, and the mortality is determined by the following equation.

$$Mortality ( \% ) = \{ 1 - number of surviving insects / 20 \} \times 100$$

### Test Example 16-1

As a result of performing a test according to Test method 16 using the following Compound of the present invention as a test compound at a predetermined concentration of 200 ppm, the following Compounds of the present invention all exhibited a mortality of 90% or more.

Compound of the present invention: 18, 20, 29, 31, 34, 35, 38, 39, 43, 45, 46, 47, 49, 50, 51, 58, 60, 62, 63, 65, 66, 67, 68, 69, 70, 71, 72, 73, 78, 79, 82, 83, 106

### Test Method 17

1 mg of the Compound of the present invention is dissolved in 10 μL of a mixed solution of xylene:DMF surfactant=4:4:1 (volume ratio), and diluted with water containing 0.02% by volume of an adjuvant to prepare a dilution A containing the Compound of the present invention at a predetermined concentration.

1 mg of the present component is dissolved in 10 μL of a mixed solution of xylene:DMF:surfactant=4:4:1 (volume ratio), and diluted with water containing 0.02% by volume of an adjuvant to prepare a dilution B containing the present component at a predetermined concentration.

The dilution A and dilution B are mixed to provide a dilution C.

Leaf pieces of cucumber cotyledons (length 1.5 cm) are accommodated in each well of a 24 well microplate, 2 apterous adults and 8 larvae of cotton aphids (Aphis gossypii) are released per well, and 20 μL of the dilution C is sprayed per well. This is defined as a treated section.

A well to which 20 μL of water containing 0.02% by volume of an adjuvant is sprayed instead of the dilution C is defined as a non-treated section.

After the dilution C is dried, the upper portion of the microplate is covered with a film sheet. After 5 days, the number of surviving insects in each well is examined.

The controlling value is calculated from the following equation.

$$Controlling value ( \% ) = \{ 1 - ( Tai ) / ( Cai ) \} \times 100$$

wherein the symbols in the equation represent the following meanings.
Cai: number of surviving insects in a non-treated section at the time of investigation
Tai: number of surviving insects in a treated section at the time of investigation

Specific dilutions C in which effects can be confirmed by test method 17 are shown in the following 1) to 5).

1) Dilution C having a concentration of the Compound of the present invention of 200 ppm and a concentration of the present component of 2000 ppm in the combination shown in the list A. In the list A, Comp X means any one compound selected from the compound groups SX1 to SX210 or the Compounds of the present invention 1 to 108.

List A: Comp X+clothianidin; Comp X+thiamethoxam; Comp X+imidacloprid; Comp X+thiacloprid; Comp X+flupyradifuron; Comp X+sulfoxaflor; Comp X+triflumezopyrime; Comp X+dicloromezotiaz; Comp X+beta cyfluthrin; Comp X+tefluthrin; Comp X+fipronil; Comp X+chlorantraniliprole; Comp X+cyantraniliprole; Comp X+tetraniliprole; Comp X+thiodicarb; Comp X+carbofuran; Comp X+fluxamethamide; Comp X+afoxolanel; Comp X+fluraranel; Comp X+brofuranilide; Comp X+abamectin; Comp X+fluopyram; Comp X+fluenesulfone; Comp X+fluazaindolidine; Comp X+thioxazafen; Comp X+flupyrimine; Comp X+mycorrhizal fungi; Comp X+Bradyrhizobium japonicum TA-11 strain; Comp X+Bacillus firmus; Comp X+Bacillus firmus I-1582 strain; Comp X+Bacillus amyloliquefaciens; Comp X+Bacillus amyloliquefaciens FZB42 strain; Comp X+pathuria nishizawae; Comp X+pathuria nishizawae Pnl strain; Comp X+Pasteuria penetrans; Comp X+tebuconazole; Comp X+prothioconazole; Comp X+metconazole; Comp X+ipconazole; Comp X+triticonazole; Comp X+difenoconazole; Comp X+imazalyl; Comp X+triazimenol; Comp X+tetraconazole; Comp X+flutriafol; Comp X+mandestrobin; Comp X+azoxystrobin; Comp X+pyraclostrobin; Comp X+trifloxystrobin; Comp X+fluoxastrobin; Comp X+picoxystrobin; Comp X+phenamidone; Comp X+metalaxyl; Comp X+metalaxyl M; Comp X+fludioxonil; Comp X+sedaxane; Comp X+penflufen; Comp X+fluxapyroxad; Comp X+benzobindiflupyr; Comp X+boscalid; Comp X+carboxine; Comp X+penthiopyrad; Comp X+flutolanyl; Comp X+captan; Comp X+thiuram; Comp X+tolclofos-methyl; Comp X+thiabendazole; Comp X+etaboxam; Comp X+mancozeb; Comp X+picarbutrazox; Comp X+oxathiapiproline; Comp X+silthiopham; and Comp X+impilfluxam.

2) Dilution C having a concentration of the Compound of the present invention of 200 ppm and a concentration of the present component of 200 ppm in the combination shown in the list A.

3) Dilution C having a concentration of the Compound of the present invention of 500 ppm and a concentration of the present component of 50 ppm in the combination shown in the list A.

4) Dilution C having a concentration of the Compound of the present invention of 500 ppm and a concentration of the present component of 5 ppm in the combination shown in the list A.

5) Dilution C having a concentration of the Compound of the present invention of 500 ppm and a concentration of the present component of 0.5 ppm in the combination shown in the list A.

### INDUSTRIAL APPLICABILITY

The Compound of the present invention exhibits an excellent control effect on harmful arthropod.

1. A compound represented by formula (I) 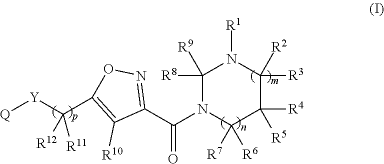
[wherein:
R¹ represents a C1-C6 chain hydrocarbon group optionally having one or more substituents selected from Group A, a C3-C6 alicyclic hydrocarbon group optionally having one or more substituents selected from Group A, a C1-C3 alkylcarbonyl group optionally having one or more halogen atoms, a C1-C3 alkoxycarbonyl group optionally having one or more halogen atoms, a C1-C3 alkylsulfonyl group optionally having one or more halogen atoms, or a hydrogen atom;
R², R³, R⁵, R⁶, and R⁷ are identical to or different from each other, and each represent a C1-C6 chain hydrocarbon group optionally having one or more substituents selected from Group A, a C3-C6 alicyclic hydrocarbon group optionally having one or more substituents selected from Group A, a C1-C3 alkoxy group optionally having one or more halogen atoms, or a hydrogen atom;
R⁴ represents a C1-C6 chain hydrocarbon group optionally having one or more substituents selected from Group A, a C3-C6 alicyclic hydrocarbon group optionally having one or more substituents selected from Group A, or a C1-C3 alkoxy group optionally having one or more halogen atoms;
R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, a phenyl group, a pyridyl group, a furyl group, a tetrahydrofuryl group, a thienyl group, a tetrahydrothienyl group, a pyridazinyl group, a pyrimidinyl group, a pyrazinyl group {the phenyl group, the pyridyl group, the furyl group, the tetrahydrofuryl group, the thienyl group, the tetrahydrothienyl group, the pyridazinyl group, the pyrimidinyl group, and the pyrazinyl group may have one or more substituents selected from Group B}, or a hydrogen atom;
m represents 0, 1, 2, or 3;
when m is 2 or 3, R² and R³ of 2 or 3 may be each independently identical to or different from each other;
n represents 1, 2, 3, or 4;
when n is 2, 3, or 4, R⁶ and R of 2, 3, or 4 may be each independently identical to or different from each other;
R² and R³ may be combined to form —(CR¹³R¹⁴)—(CR¹⁵R¹⁶)ₐ—(CR¹⁷R¹⁸)—, or (CR¹⁹R²⁰)_{b}—Z¹—(CR²¹R²²)_{c}—;
R⁴ and R⁵ may be combined to form —(CR²³R²⁴)—(CR²⁵R²⁶)_{d}—(CR²⁷R²⁸)—, or —(CR²⁹R³⁰)ₑ—Z²—(CR³¹R³²)_{f}—;
R⁶ and R⁷ may be combined to form —(CR³³R³⁴)—(CR³⁵R³⁶)_{g}—(CR³⁷R³⁸)—, or —(CR³⁹R⁴⁰)ₕ—Z³—(CR⁴¹R⁴²)ᵢ—;
R⁸ and R⁹ may be combined to form —(CR⁴³R⁴⁴)—(CR⁴⁵R⁴⁶)ⱼ—(CR⁴⁷R⁴⁸)—, or —(CR⁴⁹R⁵⁰)ₖ—Z⁴-(CR⁵¹R⁵²)ᵣ—;
a, d, g, and j each independently represent 0, 1, 2, or 3;
b, c, e, f, h, i, k, and r each independently represent 1 or 2;
Z¹, Z², Z³, and Z⁴ are identical to or different from each other, and each represent an oxygen atom, a sulfur atom, or NR⁵³;
R¹³, R¹⁴, R¹⁵, R¹⁶, R¹⁷, R¹⁸, R¹⁹, R²⁰R²¹, R²², R²³R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², and R⁵³ are identical to or different from each other, and each represent a C1-C4 alkyl group optionally having one or more halogen atoms, or a hydrogen atom;
when a, d, g, or j is 2 or 3, R¹⁵, R¹⁶, R²⁵, R²⁶, R³⁵, R³⁶, R⁴⁵, and R⁴⁶ of 2 or 3 may each independently be identical to or different from each other;
when b, c, e, f, h, i, k, or r is 2, R¹⁹, R²⁰, R²¹, R²², R²⁹, R³⁰, R³¹, R³², R³⁹, R⁴⁰, R⁴¹, R⁴², R⁴⁹, R⁵⁰, R⁵¹, and R⁵² of 2 may be each independently identical to or different from each other;
a plurality of R⁵³(s) may be each independently identical to or different from each other;
R¹⁰ represents a C1-C3 chain hydrocarbon group optionally having one or more substituents selected from Group D, a C1-C4 alkoxycarbonyl group optionally having one or more halogen atoms, a cyano group, a formyl group, a carboxy group, a carbamoyl group, a halogen atom, or a hydrogen atom;
Y represents a single bond, an oxygen atom, or —S(O)—;
when Y is a single bond, p represents 0, and Q represents a C1-C8 chain hydrocarbon group optionally having one or more substituents selected from Group F, a C3-C8 alicyclic hydrocarbon group optionally having one or more substituents selected from Group H, or a hydrogen atom;
when Y is an oxygen atom or —S(O)ᵤ—, p represents 0, 1, 2, 3, 4, 5, 6, or 7, and Q represents a C1-C8 chain hydrocarbon group optionally having one or more substituents selected from Group H, or a group selected from Group G;
u represents 0, 1, or 2;
R¹¹ and R¹² are identical to or different from each other, and each represent a C1-C4 alkyl group optionally having one or more halogen atoms, a halogen atom, or a hydrogen atom; and
when p is 1, 2, 3, 4, 5, 6, or 7, R¹¹ and R¹² of 1, 2, 3, 4, 5, 6, or 7 may be each independently identical to or different from each other,
Group A: a group consisting of a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a phenyl group, a pyridyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a pyridazinyl group, a pyrimidinyl group, a pyrazinyl group {the phenyl group, the pyridyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the pyridazinyl group, the pyrimidinyl group, and the pyrazinyl group may have one or more substituents selected from Group B}, a halogen atom, a hydroxy group, a cyano group, and CONR⁵⁴R⁵⁵;
R⁵⁴ and R⁵⁵ are identical to or different from each other, and each represent a C1-C4 alkyl group optionally having one or more halogen atoms or a hydrogen atom;
Group B: a group consisting of a C1-C4 chain hydrocarbon group optionally having one or more halogen atoms, a C1-C4 alkoxy group optionally having one or more halogen atoms, a C1-C4 alkylthio group optionally having one or more halogen atoms, a C1-C4 alkylsulfinyl group optionally having one or more halogen atoms, a C1-C4 alkylsulfonyl group optionally having one or more halogen atoms, a C1-C4 alkoxycarbonyl group optionally having one or more halogen atoms, a phenyl group optionally having one or more halogen atoms, a phenoxy group optionally having one or more halogen atoms, a cyano group, a nitro group, a carboxy group, a hydroxy group, a halogen atom, and CONR⁵⁴R⁵⁵;
Group C: a group consisting of a C1-C4 chain hydrocarbon group optionally having one or more halogen atoms, a C3-C6 alicyclic hydrocarbon group optionally having one or more substituents selected from Group E, a C1-C4 alkoxy group optionally having one or more halogen atoms, a C1-C4 alkylcarbonyl group optionally having one or more halogen atoms, a C1-C4 alkoxycarbonyl group optionally having one or more halogen atoms, a C1-C4 alkylsulfonyl group optionally having one or more halogen atoms, a phenyl group, a pyridyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a pyridazinyl group, a pyrimidinyl group, a pyrazinyl group {the phenyl group, the pyridyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the pyridazinyl group, the pyrimidinyl group, and the pyrazinyl group may have one or more substituents selected from Group B}, a halogen atom, a hydroxy group, a cyano group, and CONR⁵⁴R⁵⁵;
Group D: a group consisting of a hydroxy group and a halogen atom;
Group E: a group consisting of a C1-C4 alkoxy group optionally having one or more halogen atoms, a cyano group, a hydroxy group, and a halogen atom;
Group F: a group consisting of a C3-C8 alicyclic hydrocarbon group, an indanyl group, a 1,2,3,4-tetrahydronaphthyl group, a phenyl group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a benzofuranyl group, a benzothienyl group, a 1,3-benzodioxolyl group, a 1,4-benzodioxanyl group {the C3-C8 alicyclic hydrocarbon group, the indanyl group, the 1,2,3,4-tetrahydronaphthyl group, the phenyl group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, the benzothienyl group, the 1,3-benzodioxolyl group, and the 1,4-benzodioxanyl group may have one or more substituents selected from Group C}, a C1-C4 alkoxycarbonyl group optionally having one or more halogen atoms, a halogen atom, a cyano group, a nitro group, a carboxyl group, a hydroxyl group, and CONR⁵⁴R⁵⁵;
Group G: a group consisting of a C3-C8 alicyclic hydrocarbon group, an indanyl group, a 1,2,3,4-tetrahydronaphthyl group, a phenyl group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazoly group, a triazolyl group, an oxazolyl group, an isoxazolyl group, a thiazolyl group, an isothiazolyl group, an oxadiazolyl group, a thiadiazolyl group, a benzofuranyl group, and a benzothienyl group {the C3-C8 alicyclic hydrocarbon group, the indanyl group, the 1,2,3,4-tetrahydronaphthyl group, the phenyl group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, and the benzothienyl group may have one or more substituents selected from Group A};
Group H: a group consisting of a C3-C8 alicyclic hydrocarbon group, an indanyl group, a 1,2,3,4-tetrahydronaphthyl group, a phenyl group, a phenoxy group, a naphthyl group, a pyridyl group, a quinolyl group, a pyridazinyl group, a pyrimidinyl group, a pyrazinyl group, a furyl group, a thienyl group, a pyrrolidyl group, a pyrazolyl group, an imidazolyl group, a triazolyl group, an oxazolyl group, an isoxazolyl group, a thiazolyl group, an isothiazolyl group, an oxadiazolyl group, a thiadiazolyl group, a benzofuranyl group, a benzothienyl group, a 1,3-benzodioxolyl group, a 1,4-benzodioxanyl group {the C3-C8 alicyclic hydrocarbon group, the indanyl group, the 1,2,3,4-tetrahydronaphthyl group, the phenyl group, the phenoxy group, the naphthyl group, the pyridyl group, the quinolyl group, the pyridazinyl group, the pyrimidinyl group, the pyrazinyl group, the furyl group, the thienyl group, the pyrrolidyl group, the pyrazolyl group, the imidazolyl group, the triazolyl group, the oxazolyl group, the isoxazolyl group, the thiazolyl group, the isothiazolyl group, the oxadiazolyl group, the thiadiazolyl group, the benzofuranyl group, the benzothienyl group, the 1,3-benzodioxolyl group, and the 1,4-benzodioxanyl group may have one or more substituents selected from Group C}, a C1-C4 alkoxycarbonyl group optionally having one or more halogen atoms, a halogen atom, a cyano group, a nitro group, a carboxyl group, a hydroxyl group, and CONR⁵⁴R⁵⁵]
or a salt thereof.

2. The compound or a salt thereof according to claim 1, wherein
R¹ represents a C1-C6 chain hydrocarbon group optionally having one or more substituents selected from Group A, a C3-C6 alicyclic hydrocarbon group optionally having one or more substituents selected from Group A, or a hydrogen atom.

3. The compound or a salt thereof according to claim 1, wherein
R⁸ and R⁹ are identical to or different from each other, and each represent a C1-C20 chain hydrocarbon group optionally having one or more substituents selected from Group A, a C3-C6 alicyclic hydrocarbon group optionally having one or more substituents selected from Group A, or a hydrogen atom.

4. The compound or a salt thereof according to claim 1, wherein
R¹⁰ represents a C1-C3 chain hydrocarbon group optionally having one or more substituents selected from Group D or a hydrogen atom.

5. The compound or a salt thereof according to claim 1, wherein
n represents 1.

6. The compound or a salt thereof according to claim 1, wherein
m represents 0 or 1.

7. The compound or a salt thereof according to claim 1, wherein
m represents 0.

8. A composition for controlling a harmful arthropod comprising the compound or a salt thereof according to claim 1, and an inert carrier.

9. A composition comprising one or more ingredients selected from the group consisting of Group (a), Group (b), Group (c), and Group (d), and the compound or a salt thereof according to claim 1:
Group (a): a group consisting of insecticidal active ingredients, miticidal active ingredients and nematicidal active ingredients;
Group (b): fungicidal active ingredients;
Group (c): plant growth modulating ingredients; and
Group (d): repellent ingredients.

10. A method for controlling a harmful arthropod, which comprises applying an effective amount of the compound or a salt thereof according to claim 1 to a harmful arthropod or a habitat where the harmful arthropod lives.

11. A seed or vegetative reproductive organ carrying an effective amount of the compound or a salt thereof according to claim 1.

12. A method for controlling a harmful arthropod, which comprises applying an effective amount of the composition according to claim 9 to a harmful arthropod or a habitat where the harmful arthropod lives.

13. A seed or vegetative reproductive organ carrying an effective amount of the composition according to claim 9.
